(19) United States
(12) Patent Application Publication
(10) Pub. No.: US 2008/0292588 A1
(43) Pub. Date: Nov. 27, 2008

(54) 1-METHYL-BENZO[1,2,4]THIADIAZINE 1-OXIDE DERIVATIVES

(75) Inventors: Yuefen Zhou, San Diego, CA (US); Thomas Bertolini, San Diego, CA (US); Liansheng Li, San Diego, CA (US)

Correspondence Address: CONNOLLY BOVE LODGE & HUTZ LLP, 1875 EYE STREET, N.W., SUITE 1100, WASHINGTON, DC, 20006, US

(73) Assignee: Anadys Pharmaceuticals, Inc., San Diego, CA (US)

(21) Appl. No.: 12/121,928

(22) Filed: May 16, 2008

Related U.S. Application Data
(60) Provisional application No. 60/924,491, filed on May 17, 2007.
(60) Provisional application No. 60/924,492, filed on May 17, 2007.
(60) Provisional application No. 60/924,493, filed on May 17, 2007.
(60) Provisional application No. 60/924,494, filed on May 17, 2007.
(60) Provisional application No. 60/972,105, filed on Sep. 13, 2007.

(51) Int. Cl. A61K 31/549; C07D 285/24; A61K 38/21; A61P 31/14

(52) U.S. Cl. 424/85.6; 544/12; 514/223.5; 514/223.2; 424/85.7

(57) ABSTRACT

The invention is directed to 1-methyl-benzo[1,2,4]thiadiazine 1-oxide derivatives and pharmaceutical compositions containing such compounds that are useful in treating infections by hepatitis C virus.

### CROSS REFERENCE TO RELATED APPLICATIONS

This application claims the benefit of U.S. provisional application No. 60/924,491 filed on May 17, 2007; U.S. provisional application No. 60/924,492 filed on May 17, 2007; U.S. provisional application No. 60/924,493 filed on May 17, 2007; U.S. provisional application No. 60/924,494 filed on May 17, 2007; and U.S. provisional application No. 60/972,105 filed on Sep. 13, 2007. The entirety of these applications is hereby incorporated by reference.

### FIELD OF THE INVENTION

The invention is directed to 1-methyl-benzo[1,2,4]thiadiazine 1-oxide derivatives and pharmaceutical compositions containing such compounds that are useful in treating infections by hepatitis C virus.

### BACKGROUND OF THE INVENTION

Hepatitis C is a major health problem world-wide. The World Health Organization estimates that 170 million people are chronic carriers of the hepatitis C virus (HCV), with 4 million carriers in the United States alone. In the United States, HCV infection accounts for 40% of chronic liver disease and HCV disease is the most common cause for liver transplantation. HCV infection leads to a chronic infection and about 70% of persons infected will develop chronic histological changes in the liver (chronic hepatitis) with a 10-40% risk of cirrhosis and an estimated 4% lifetime risk of hepatocellular carcinoma. The CDC estimates that each year in the United States there are 35,000 new cases of HCV infection and approximately ten thousand deaths attributed to HCV disease.

The current standard of care is a pegylated interferon/ribavirin combination at a cost of approximately $31,000/year. These drugs have difficult dosing problems and side-effects that preclude their use in almost half of diagnosed patients. Pegylated interferon treatment is associated with menacing flu-like symptoms, irritability, inability to concentrate, suicidal ideation, and leukocytopenia. Ribavirin is associated with hemolytic anemia and birth defects.

The overall response to this standard therapy is low; approximately one third of patients do not respond. Of those who do respond, a large fraction relapses within six months of completing 6-12 months of therapy. As a consequence, the long-term response rate for all patients entering treatment is only about 50%. The relatively low response rate and the significant side-effects of current therapy anti-HCV drug treatments, coupled with the negative long term effects of chronic HCV infection, result in a continuing medical need for improved therapy. Antiviral pharmaceuticals to treat RNA virus diseases like HCV are few, and as described above are often associated with multiple adverse effects.

A number of recent publications have described NS5B inhibitors useful in the treatment of hepatitis C infection. See, e.g., U.S. Patent Application Publication No. US 2006/0189602 (disclosing certain pyridazinones); U.S. Patent Application Publication No. US 2006/0252785 (disclosing selected heterocyclics); and International Publication Nos. WO 03/059356, WO 2002/098424, and WO 01/85172 (each describing a particular class of substituted thiadiazines).

While there are, in some cases, medicines available to reduce disease symptoms, there are few drugs to effectively inhibit replication of the underlying virus. The significance and prevalence of RNA virus diseases, including but not limited to chronic infection by the hepatitis C virus, and coupled with the limited availability and effectiveness of current antiviral pharmaceuticals, have created a compelling and continuing need for new pharmaceuticals to treat these diseases.

### SUMMARY OF THE INVENTION

The present invention describes novel 1-methyl-benzo[1,2,4]thiadiazine 1-oxide derivatives and pharmaceutically acceptable salts thereof, which are useful in treating or preventing a hepatitis C virus infection in a patient in need thereof comprising administering to the patient a therapeutically or prophylactically effective amount of a 1-methyl-benzo[1,2,4]thiadiazine 1-oxide compound.

In a general aspect, the invention relates to compounds of Formula I

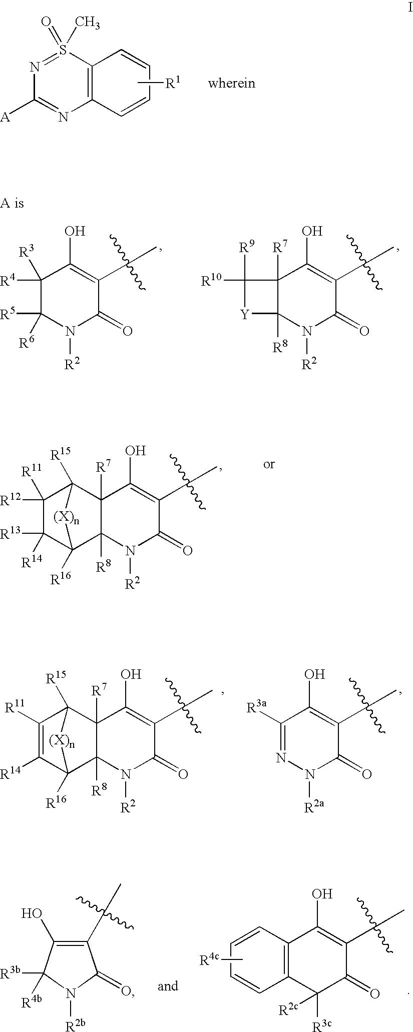

wherein
R¹ is 1-4 moieties selected independently from H, halo, nitro, aryl, heterocyclyl, —CHR¹⁷—S(O)₂R¹⁸, —NR¹⁸R¹⁹, —NR¹⁷S(O)₂R¹⁸, and —NR¹⁷S(O)₂NR^{18a}R^{19a}, wherein R¹⁷, R¹⁸, R¹⁹, R^{18a}, and R^{19a}, are independently H, C₁-C₆ alkyl, C₃-C₈ cycloalkyl, aryl, or heterocyclyl, or R^{18a} and R^{19a} combine with the atom(s) to which they are attached to form a 5- or 6-membered heterocyclyl ring,
R² is H, C₁-C₈ alkyl, C₂-C₆ alkenyl, C₁-C₆ alkylamine, C₁-C₆ dialkylamine, C₃-C₈ cycloalkyl, —C₁-C₆ alkylene(C₃-C₈ cycloalkyl), —C₁-C₆ alkylene(aryl), —C₁-C₆ alkylene(heterocyclyl), aryl, or heterocyclyl,
R³, R⁴, R⁵ and R⁶ are independently H, halo, cyano, hydroxyl, amino, C₁-C₆ alkyl, C₁-C₆ alkoxy, C₁-C₆ hydroxyalkyl, C₂-C₆ alkenyl, C₂-C₆ alkynyl, C₁-C₆ alkylamine, C₁-C₆ dialkylamine, C₃-C₈ cycloalkyl, C₁-C₆ alkylene(cycloalkyl), C₁-C₆ alkylene(aryl), C₁-C₆ alkylene(heterocyclyl), aryl, heterocyclyl, or R³ and R⁴ or R⁵ and R⁶ can combine with the atom(s) to which they are attached to form a 3- to 6-membered cycloalkyl spiro ring,
or R² and R⁵, or R² and R⁶ can combine with the atom(s) to which they are attached to form a 5- or 6-membered heterocyclyl ring,
   Y is —(CR²⁰R²¹)ₘ, wherein m is 2, 3, 4 or 5,
   n is 1 or 2,
   wherein when n is 1, X is O or —CR²²R²³, and when n is 2, X is —CR²²R²³—CR²⁴R²⁵, R⁷, R⁸, R⁹, R¹⁰, R¹¹, R¹², R¹³, R¹⁴, R¹⁵, R¹⁶, R²⁰, R²¹, R²², R²³, R²⁴ and R²⁵ are independently H, C₁-C₆ alkyl, or halo, or R⁷ and R⁹ combine with the atoms to which they are attached to form a 3-membered cycloalkyl ring, or R²² and R²⁴ combine with the atoms to which they are attached to form a 3-membered cycloalkyl ring,
   R^{2a} is C₁-C₆ alkyl, C₃-C₈ cycloalkyl, —C₁-C₆ alkylene(C₃-C₈ cycloalkyl), —C₁-C₆ alkylene(aryl), —C₁-C₆ alkylene(heterocyclyl), aryl, or heterocyclyl,
   R^{3a} is H, halo, C₁-C₆ alkyl, C₁-C₆ alkoxy, C₁-C₆ haloalkyl, C₁-C₆ hydroxyalkyl, C₂-C₆ alkenyl, C₃-C₈ cycloalkyl, aryl, heterocyclyl, NR¹⁸R¹⁹,
   R^{2b} is C₁-C₇ alkyl, C₂-C₆ alkenyl, C₃-C₈ cycloalkyl, —C₁-C₆ alkylene(C₃-C₈ cycloalkyl), —C₁-C₆ alkylene(aryl), —C₁-C₆ alkylene(heterocyclyl), aryl, or heterocyclyl,
   R^{3b} and R^{4b} are independently selected from H, C₁-C₆ alkyl, C₃-C₈ cycloalkyl, C₁-C₆ alkylene(aryl), aryl, heterocyclyl, or R^{3b} and R^{4b} combine with the C atom to which they are attached to form a 3-, 4-, 5- or 6-membered heterocyclyl ring that may contain hetero-atoms such as N on the ring,
   R^{2c} and R^{3c} are independently H, C₁-C₆ alkyl, C₁-C₆ alkenyl, C₁-C₆ haloalkyl, C₁-C₆ hydroxyalkyl, —C₁-C₆ alkylene(C₃-C₈ cycloalkyl), —C₁-C₆ alkylene(aryl), or —C₁-C₆ alkylene(heterocyclyl) having 1, 2, or 3 N, O, or S atoms, or R^{2c} and R^{3c} together with the carbon atom to which they are attached to form a monocyclic ring selected from the group consisting of cycloalkyl and cycloalkenyl, or R^{3c} is —NHR²⁶ wherein R²⁶ is selected from C₁-C₆ alkyl, —C₁-C₆ alkylene(aryl), C₁-C₆ alkoxy, C₁-C₆ alkylene-CO₂R¹⁸, COR¹⁸, CO₂R¹⁸, CONH₂ and SO₂R¹⁸,
   R^{4c} is independently H, halo, OH, CN, C₁-C₆ alkyl, C₁-C₆ alkoxy, C₁-C₆ haloalkyl, C₁-C₆ hydroxyalkyl, C₂-C₆ alkenyl, C₂-C₆ alkynyl, COOH, CONR^{18a}R^{19a} and COOR¹⁸, wherein the above alkyl, alkylene, alkenyl, alkynyl, aryl, cycloalkyl, or heterocyclyl moieties provided in R¹, R², R^{2a}, R^{2b}, R^{2c}, R³, R^{3a}, R^{3b}, R^{3c}, R⁴, R^{4b}, R^{4c}, R⁵, R⁶, R⁷, R⁸, R⁹, R¹⁰, R¹¹, R¹², R¹³, R¹⁴, R¹⁵, R¹⁶, R¹⁷, R¹⁸, R^{18a}, R¹⁹, R^{19a}, R²⁰, R²¹, R²², R²³, R²⁴, R²⁵ and R²⁶ are each optionally and independently substituted by 1-3 substituents selected from
   alkylamine,
   amino,
   aryl, cycloalkyl, heterocyclyl, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms,
   carboxyl,
   cyano,
   halo,
   hydroxy,
   nitro,
   —C(O)OH, —C(O)₂—(C₁-C₆ alkyl), —C(O)₂—(C₃-C₈ cycloalkyl), —C(O)₂-(aryl), —C(O)₂— (heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkyl), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkylene)cycloalkyl,

wherein each of the above optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₂-C₆ alkenyl, and C₁-C₆ hydroxyalkyl, wherein each alkyl is optionally substituted by one or more halo substituents, or a pharmaceutically acceptable salt, hydrate, tautomer or stereoisomer thereof.

In one embodiment, the invention relates to compounds of Formula I wherein R¹ is H or —NR⁷S(O)₂R¹⁸, wherein R¹⁷ and R¹⁸ are independently H, C₁-C₆ alkyl, C₃-C₈ cycloalkyl, aryl, or heterocyclyl, or R¹⁷ and R¹⁸ combine with the atom(s) to which they are attached to form a 5- or 6-membered heterocyclyl ring.

In another embodiment, the invention relates to compounds of Formula I wherein R¹ is selected from

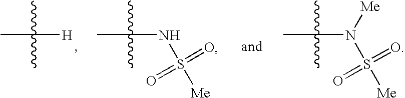

In one embodiment, the invention relates to compounds of Formula I wherein R² is selected from C₁-C₆ alkyl, C₃-C₈ cycloalkyl, —C₁-C₆ alkylene(C₃-C₈ cycloalkyl), —C₁-C₆ alkylene(aryl), and —C₁-C₆ alkylene(heterocyclyl).

In another embodiment, the invention relates to compounds of Formula I wherein R² is selected from

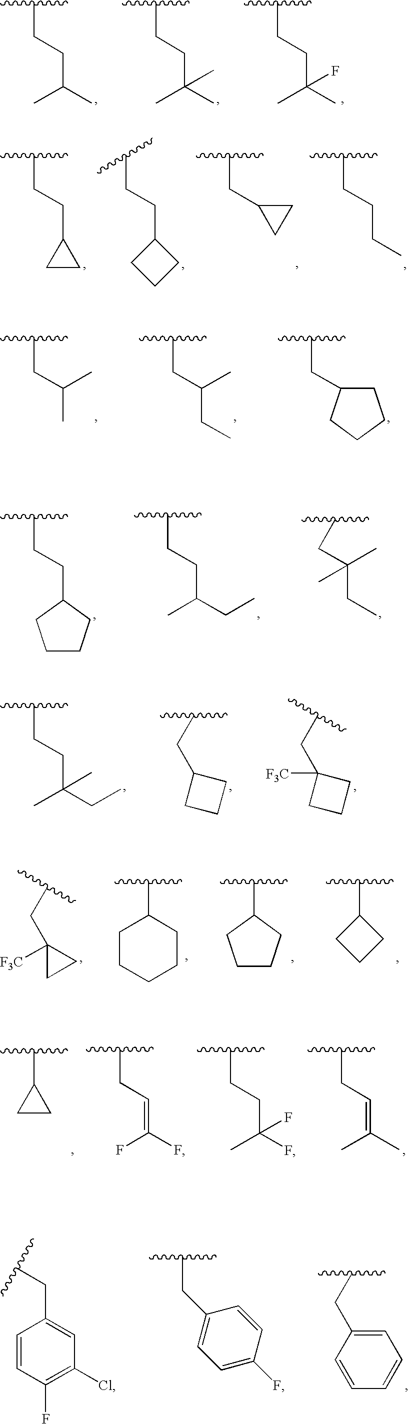

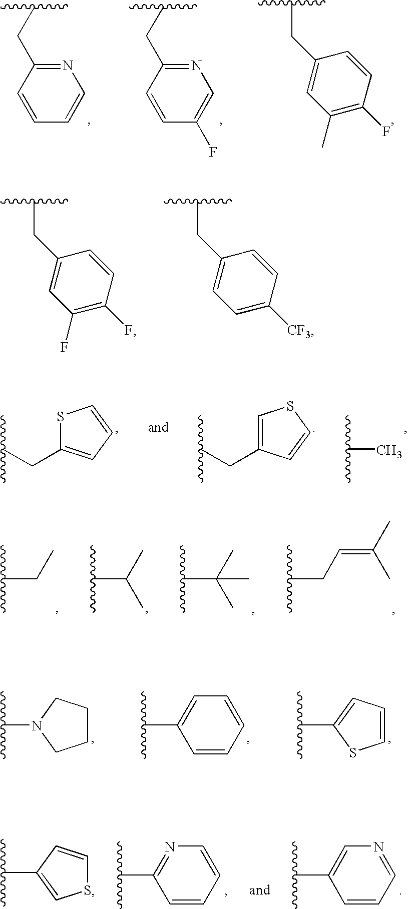

In a further embodiment, R² is selected from

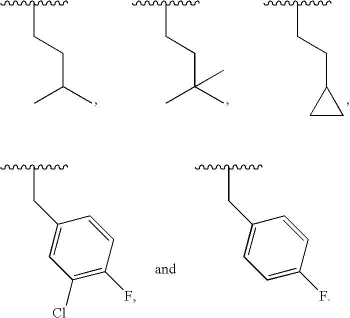

In one embodiment, R², R³, R⁴, R³, R⁶, R⁷, R⁸, R¹¹, R¹², R¹³, R¹⁴, R¹⁵, R¹⁶, R²², and R²³ are independently H, or C₁-C₆ alkyl, optionally substituted. In one embodiment, R^{2b}, R^{3b}, and R^{4b} are independently H or C₁-C₆ alkyl, optionally substituted.

In another embodiment, R^{2c}, R^{3c}, and R^{4c} are independently H, C₁-C₆ alkyl, or C₃-C₈ cycloalkyl, optionally substituted.

In one embodiment, the invention relates to compounds of Formula I wherein R³, R⁴, R⁵, and R⁶ are independently selected from C₁-C₆ alkyl, C₃-C₈ cycloalkyl, aryl, and heterocyclyl.

In another embodiment, the invention relates to compounds of Formula I wherein R³, R⁴, R⁵ and R⁶ are independently selected from

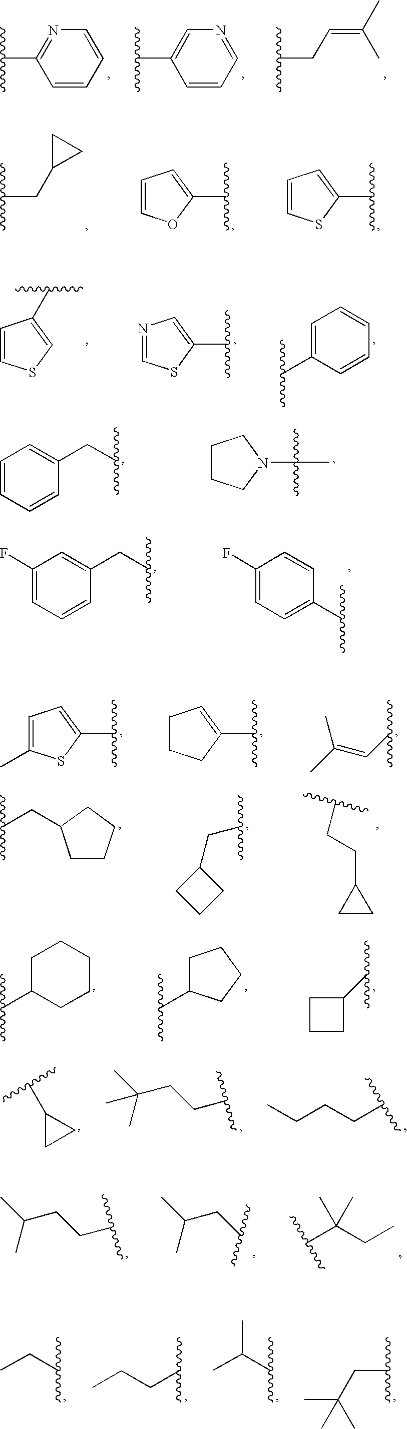

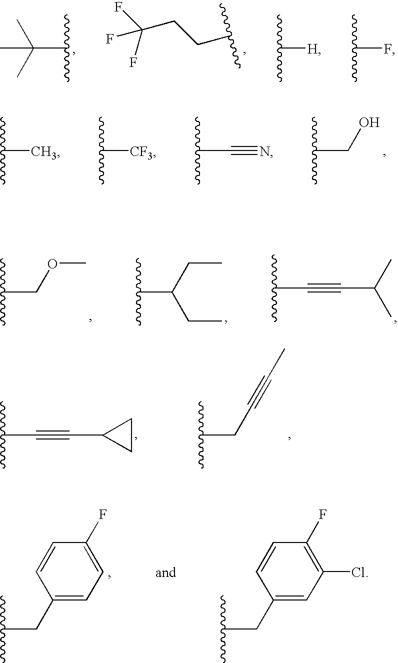

or R³ and R⁴ or R⁵ and R⁶ can combine with the atom(s) to which they are attached to form

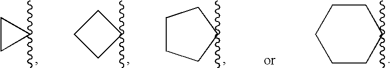

In a further embodiment, the invention relates to compounds of Formula I wherein R³, R⁴, R⁵ and R⁶ are independently selected from

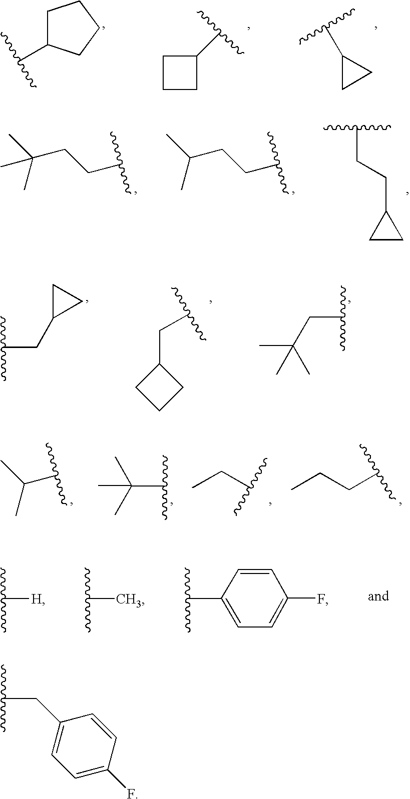

or R³ and R⁴ or R⁵ and R⁶ can combine with the atom(s) to which they are attached to form

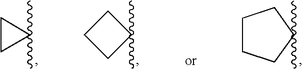

In another embodiment, the invention relates to compounds of Formula I wherein R⁷, R⁸, R⁹, R¹⁰, R¹¹, R¹², R¹³, R¹⁴, R¹⁵, and R¹⁶ are independently selected from H and Me.

In one embodiment, the invention relates to compounds of Formula I wherein R^{2a} is selected from C₁-C₆ alkyl, C₃-C₈ cycloalkyl, —C₁-C₆ alkylene(C₃-C₈ cycloalkyl), —C₁-C₆ alkylene(aryl), and —C₁-C₆ alkylene(heterocyclyl).

In another embodiment, the invention relates to compounds of Formula I wherein R^{2a} is selected from

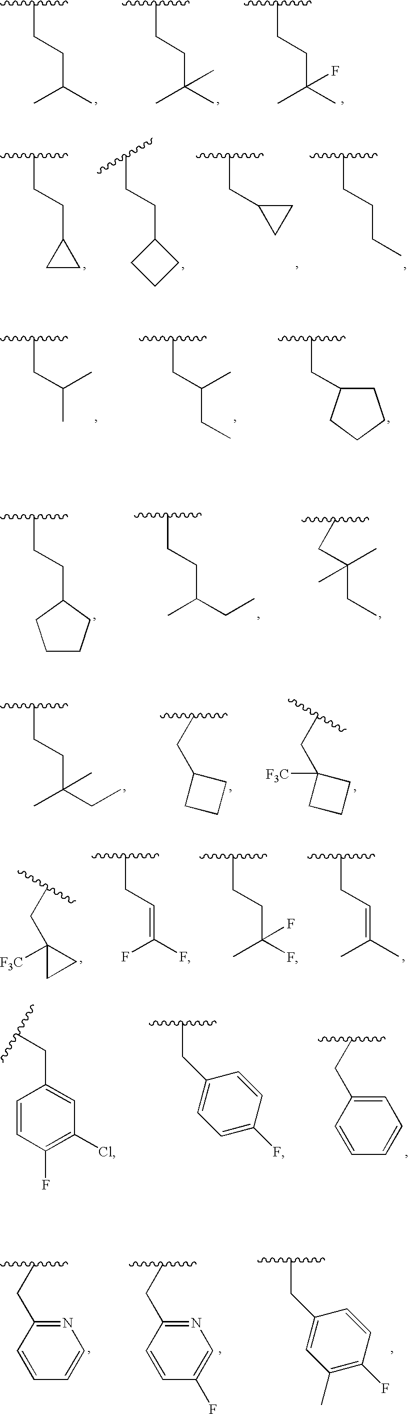

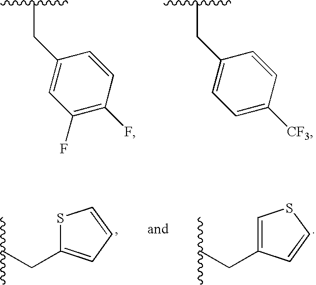

In a further embodiment, R^{2a} is selected from

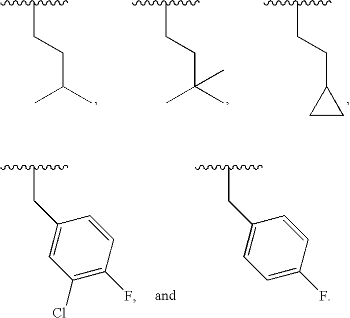

In one embodiment, the invention relates to compounds of Formula I wherein R^{3a} is selected from C₁-C₆ alkyl, C₃-C₈ cycloalkyl, aryl, and heterocyclyl.

In another embodiment, the invention relates to compounds of Formula I wherein R^{3a} is selected from

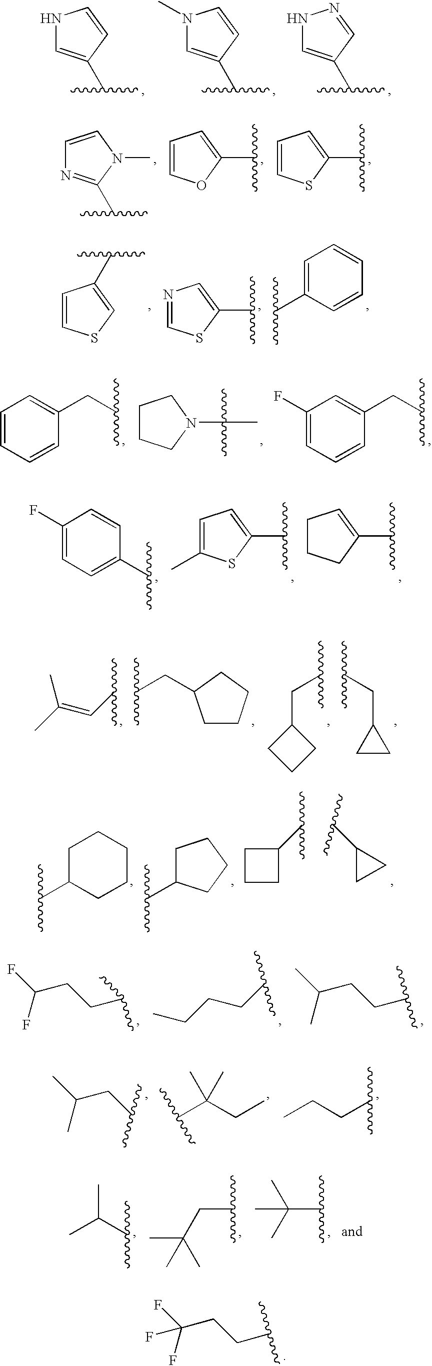

In a further embodiment, the invention relates to compounds of Formula I wherein R^{3a} is selected from

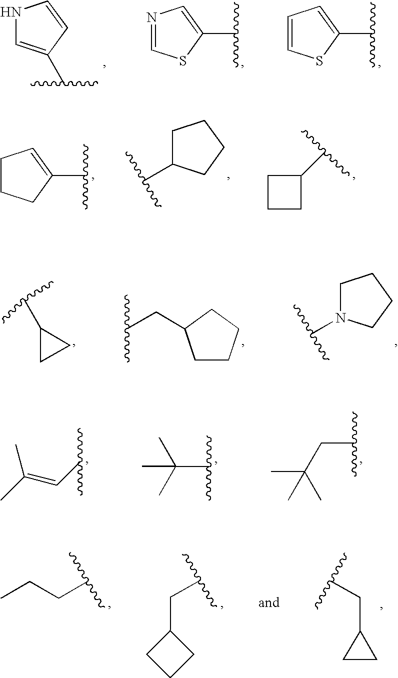

In one embodiment, the invention relates to compounds of Formula I wherein R^{2b} is selected from C₁-C₆ alkyl, C₃-C₈ cycloalkyl, —C₁-C₆ alkylene(C₃-C₈ cycloalkyl), —C₁-C₆ alkylene(aryl), and —C₁-C₆ alkylene(heterocyclyl).

In another embodiment, the invention relates to compounds of Formula I wherein R^{2b} is selected from

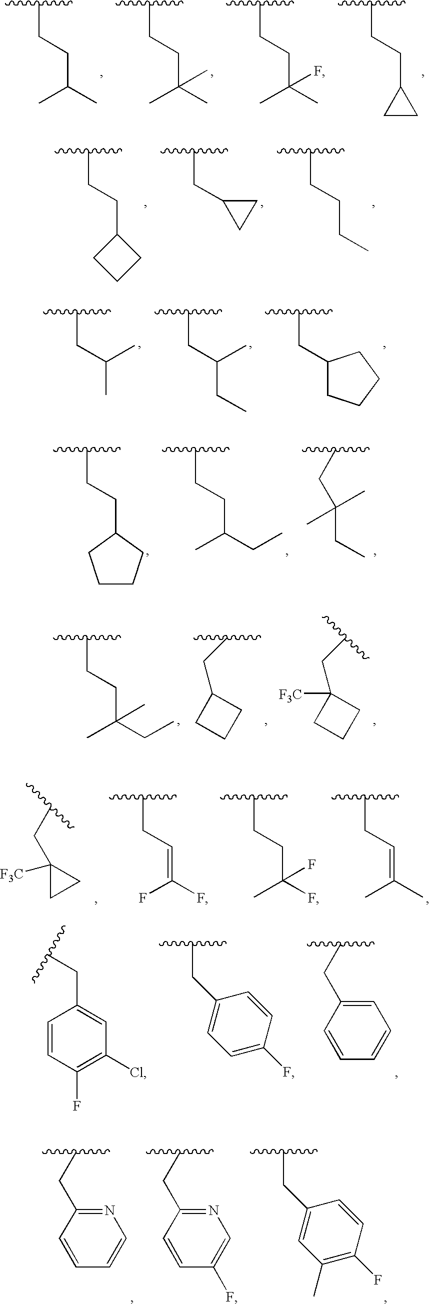

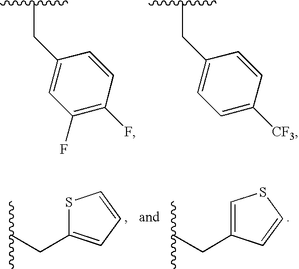

In a further embodiment, R^{2b} is selected from

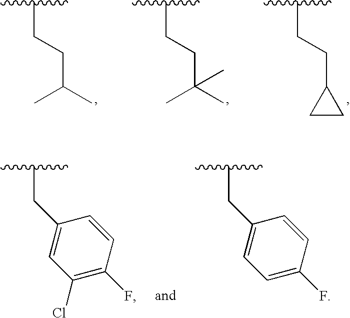

In one embodiment, the invention relates to compounds of Formula I wherein R^{3b} and R^{4b} are independently selected from C₁-C₆ alkyl, C₃-C₈ cycloalkyl, aryl, and heterocyclyl.

In another embodiment, the invention relates to compounds of Formula I wherein R^{3b} and R^{4b} are independently selected from

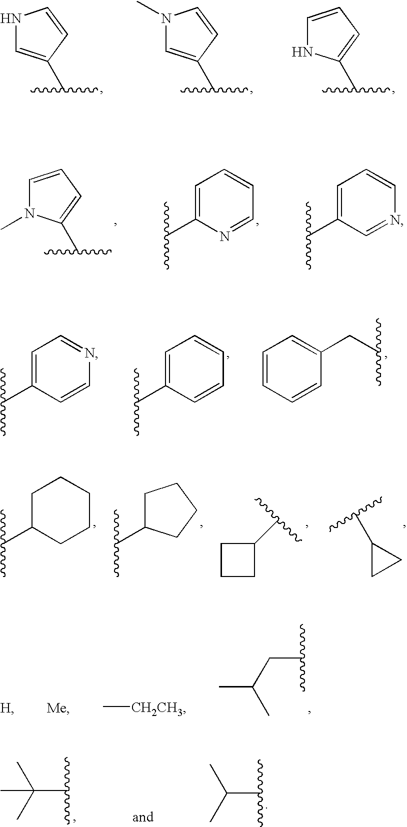

In a further embodiment, the invention relates to compounds of Formula I wherein R^{3b} and R^{4b} are independently selected from

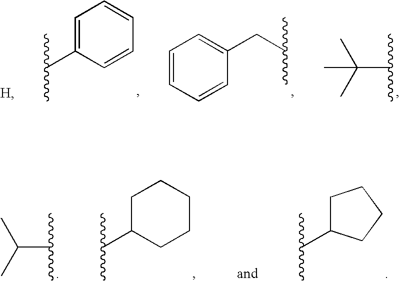

In one embodiment, the invention relates to compounds of Formula I wherein R^{2c} and R^{3c} are independently selected from C₁-C₆ alkyl, —C₁-C₆ alkylene(C₃-C₈ cycloalkyl), —C₁-C₆ alkylene(aryl), and —C₁-C₆ alkylene(heterocyclyl) having 1, 2, or 3 N, O, or S atoms.

In another embodiment, the invention relates to compounds of Formula I wherein R^{2c} and R^{3c} are independently selected from

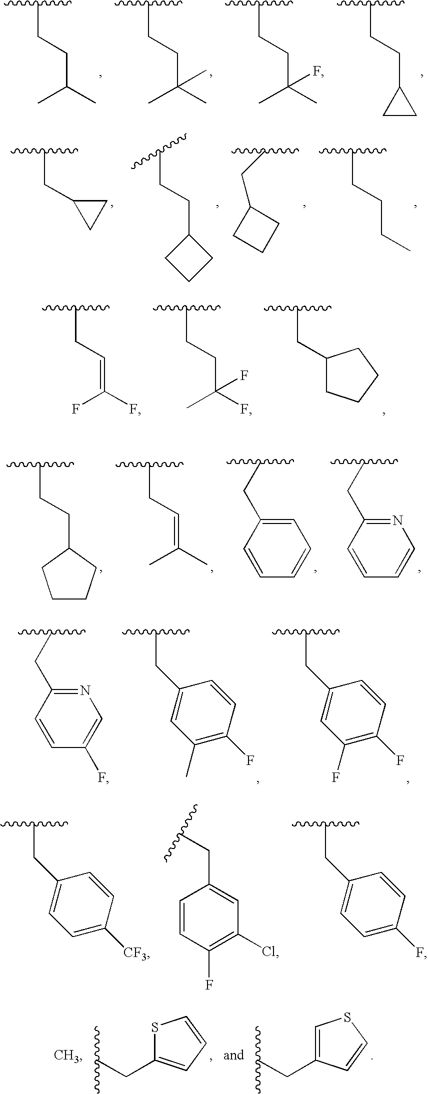

In a further embodiment, R^{2c} and R^{3c} are selected from

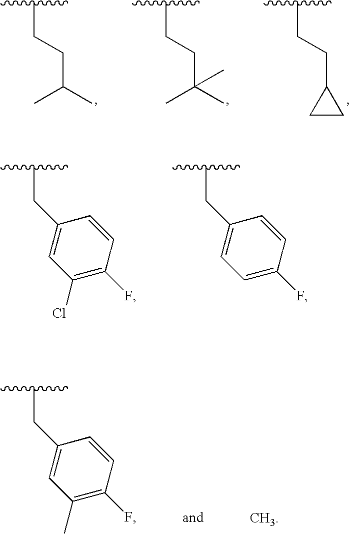

In one embodiment, the invention relates to compounds of Formula I wherein R^{4c} is selected from H, halo, OH, CN, C₁-C₆ alkyl, COOH, CONHR^{18a}R^{19a} and COOR¹⁸.

In another embodiment, the invention relates to compounds of Formula I wherein R^{4c} is selected from H, F, CH₃, OH, CN, COOH, CO₂Me and CONH₂. In a further embodiment, the invention relates to compounds of Formula I wherein R^{4c} is selected from H, F, and OH.

In one embodiment, the invention relates to compounds of Formula I wherein R^{3c} is —NHR²⁶ and R²⁶ is selected from C₁-C₆ alkyl, —C₁-C₆ alkylene(aryl) and C₁-C₆ alkoxy.

In another embodiment, the invention relates to compounds of Formula I wherein R^{3c} is —NHR²⁶ and R²⁶ is selected from OCH₃, COCH₃, COPh, CO₂CH₃, CO₂CH₂Ph, CONH₂, SO₂CH₃, CH₂CH(CH₃)₂, CH₂(c-Pr), CH₂Ph and CH₂CO₂Et. In yet another embodiment, R²⁶ is OCH₃ or CH₂Ph. The notation c-Pr identifies cyclopropyl, such that CH₂(c-Pr) is:

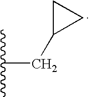

In one aspect, the invention is compounds of Formula I wherein A is selected from the group consisting of:

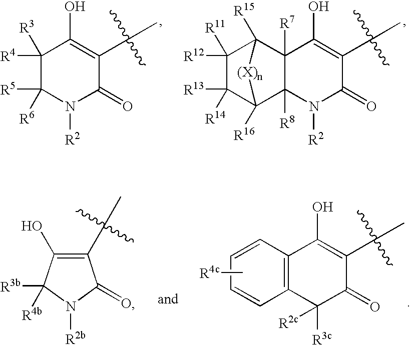

In another embodiment, the invention relates to compounds of Formula I wherein A can be selected from

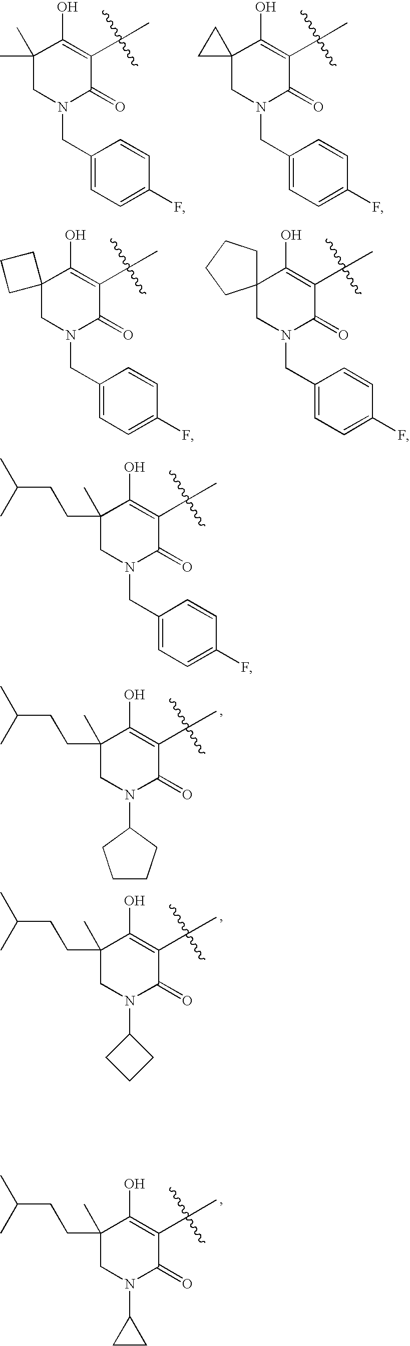

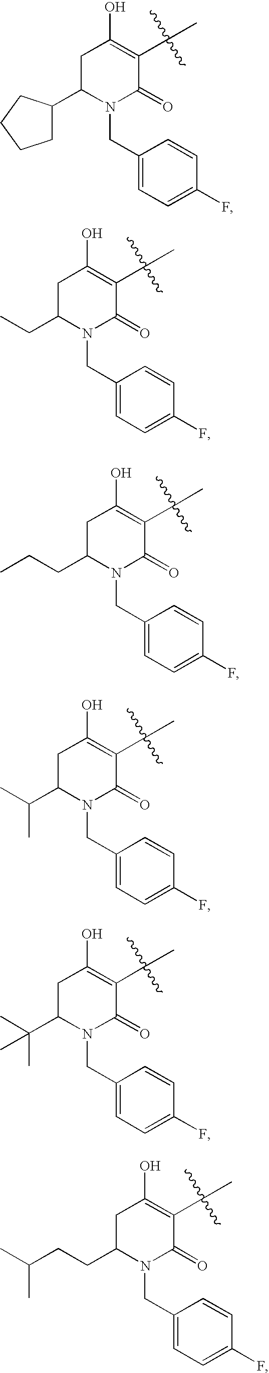

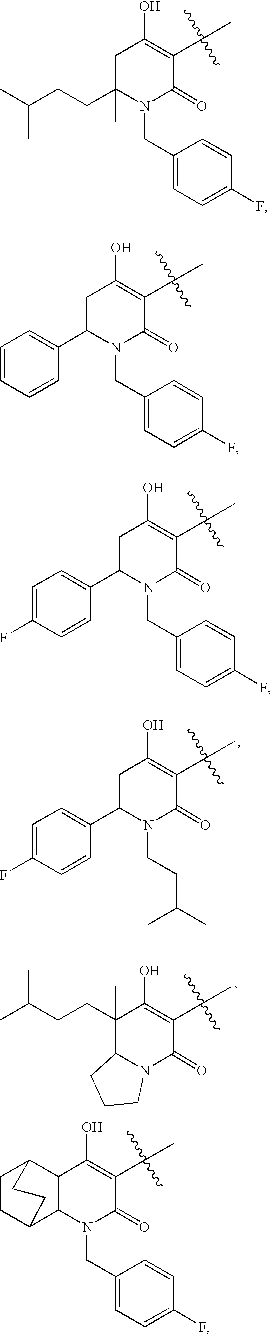

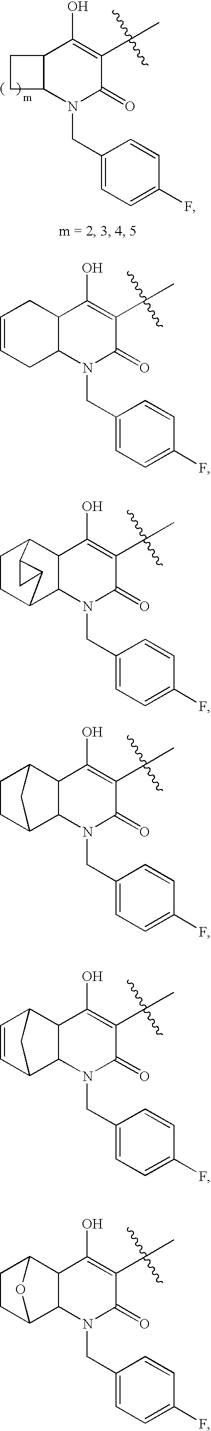

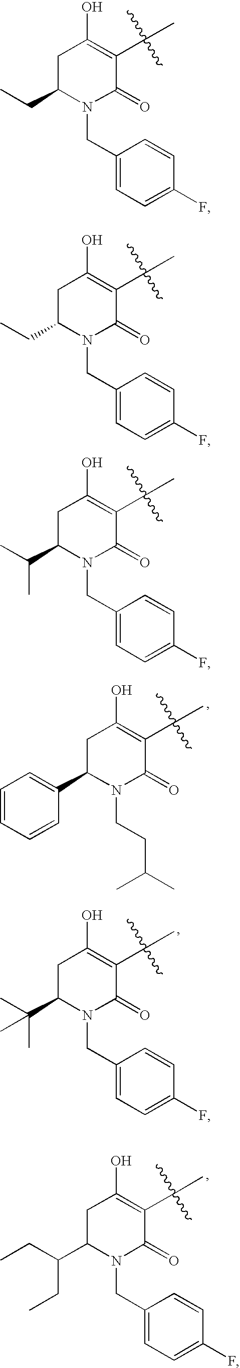

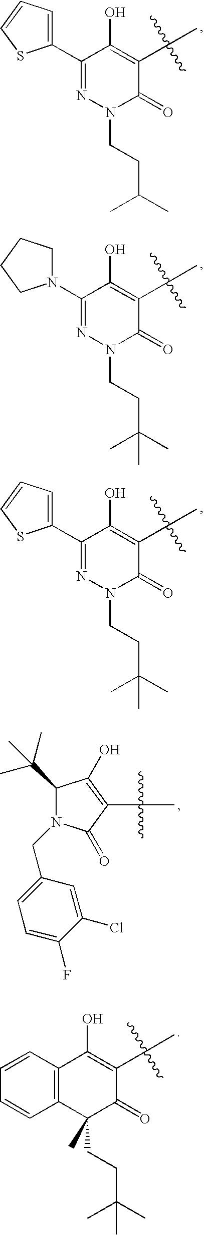

In one embodiment, the invention relates to compounds of Formula I selected from

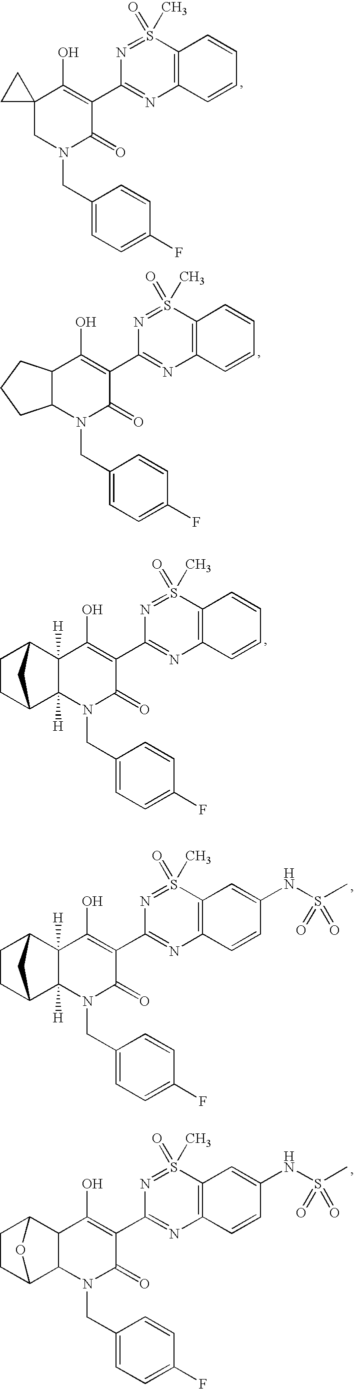

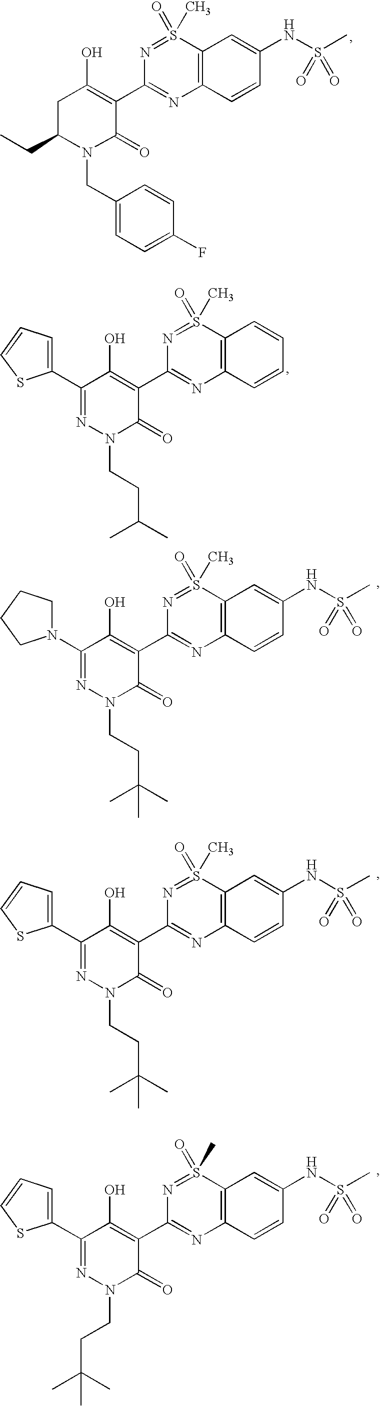

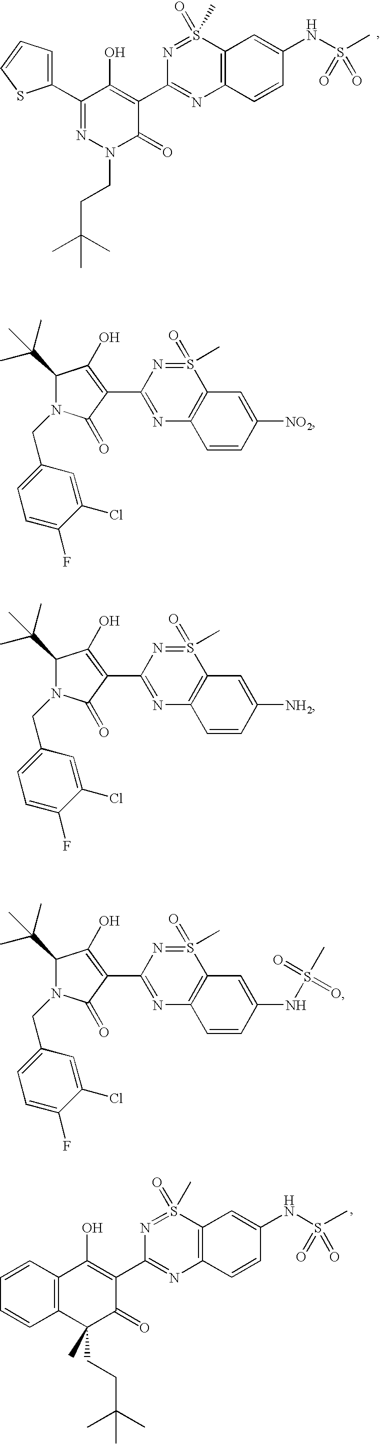

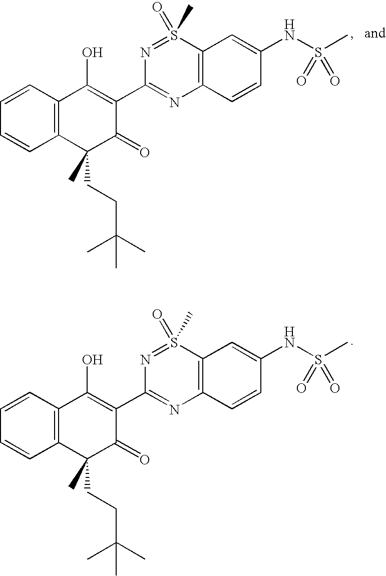

The invention is also directed to pharmaceutically acceptable salts and pharmaceutically acceptable solvates of the compounds of Formula I. Advantageous methods of making the compounds of Formula I are also described.

The invention is also directed to a method of inhibiting hepatitis C virus replication comprising exposing hepatitis C virus to a therapeutically effective amount of a compound of Formula I.

In one aspect, the invention encompasses a method for treating or preventing hepatitis C virus infection in a mammal in need thereof, preferably in a human in need thereof, comprising administering to the patient a therapeutically or prophylactically effective amount of a Formula I compound. In one embodiment, the invention encompasses a method for treating or preventing hepatitis C virus infection by administering to a patient in need thereof a therapeutically or prophylactically effective amount of a Formula I compound that is an inhibitor of HCV NS5B polymerase.

In another aspect, the invention encompasses a method for treating or preventing hepatitis C virus infection in a patient in need thereof, comprising administering to the patient a therapeutically or prophylactically effective amount of a compound of Formula I and a pharmaceutically acceptable excipient, carrier, or vehicle.

In another aspect, the invention encompasses a method for treating or preventing hepatitis C virus infection in a patient in need thereof, comprising administering to the patient a therapeutically or prophylactically effective amount of a compound of Formula I and an additional therapeutic agent, preferably an additional antiviral agent or an immunomodulatory agent.

### DETAILED DESCRIPTION OF THE INVENTION

Where the following terms are used in this specification, they are used as defined below:

The terms “comprising,” “having” and “including” are used herein in their open, non-limiting sense.

The term “alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

The term “alkylene”, as used herein, unless otherwise indicated, includes a divalent radical derived from alkyl, as exemplified by —CH₂CH₂CH₂CH₂—.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “Me” means methyl, “Et” means ethyl, and “Ac” means acetyl.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

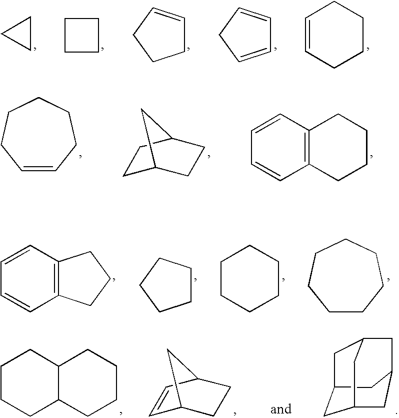

The term “aryl”, as used herein, unless otherwise indicated, includes an organic radical derived from an aromatic hydrocarbon by removal of one hydrogen, such as phenyl or naphthyl.

The term “heterocyclic” or “heterocyclyl”, as used herein, unless otherwise indicated, includes aromatic (e.g., heteroaryls) and non-aromatic heterocyclic groups containing one to three heteroatoms each selected from O, S and N, wherein each heterocyclic group has up to 10 atoms in its ring system, and with the proviso that the ring of said group does not contain two adjacent O atoms. Non-aromatic heterocyclic groups include groups having at least 3 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other illustrative examples of 4-10 membered heterocyclic are derived from, but not limited to, the following:

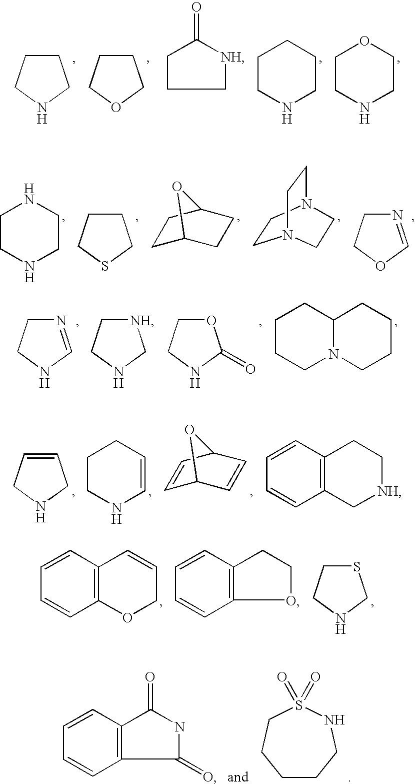

Unless defined otherwise, “alkyl,” “alkylene,” “alkenyl,” “alkynyl,” “aryl,” “cycloalkyl,” or “heterocyclyl” are each optionally and independently substituted by 1-3 substituents selected from alkanoyl, alkylamine, amino, aryl, cycloalkyl, heterocyclyl, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms, carboxyl, cyano, halo, hydroxy, nitro, —C(O)OH, —C(O)₂—(C₁-C₆ alkyl), —C(O)₂—(C₃-C₈ cycloalkyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkyl), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkylene)cycloalkyl, wherein each of these optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents, e.g., CF₃.

The term “immunomodulator” refers to natural or synthetic products capable of modifying the normal or aberrant immune system through stimulation or suppression.

The term “preventing” refers to the ability of a compound or composition of the invention to prevent a disease identified herein in patients diagnosed as having the disease or who are at risk of developing such disease. The term also encompasses preventing further progression of the disease in patients who are already suffering from or have symptoms of such disease.

The term “patient” or “subject” means an animal (e.g., cow, horse, sheep, pig, chicken, turkey, quail, cat, dog, mouse, rat, rabbit, guinea pig, monkeys, etc.) or a mammal, including chimeric, cloned and transgenic animals and mammals. In the treatment or prevention of HCV infection, the term “patient” or “subject” preferably means a monkey or a human, most preferably a human. In a specific embodiment the patient or subject is infected by or exposed to the hepatitis C virus. In certain embodiments, the patient is a human infant (age 0-2), child (age 2-17), adolescent (age 12-17), adult (age 18 and up) or geriatric (age 70 and up) patient. In addition, the patient includes immunocompromised patients such as HIV positive patients, cancer patients, patients undergoing immunotherapy or chemotherapy. In a particular embodiment, the patient is a healthy individual, i.e., not displaying symptoms of other viral infections.

The term a “therapeutically effective amount” refers to an amount of the compound of the invention sufficient to provide a benefit in the treatment or prevention of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

The term a “prophylactically effective amount” refers to an amount of a compound of the invention or other active ingredient sufficient to result in the prevention of infection, recurrence or spread of viral infection. A prophylactically effective amount may refer to an amount sufficient to prevent initial infection or the recurrence or spread of the infection or a disease associated with the infection. Used in connection with an amount of a compound of the invention, the term preferably encompasses a non-toxic amount that improves overall prophylaxis or enhances the prophylactic efficacy of or synergies with another prophylactic or therapeutic agent.

The term “in combination” refers to the use of more than one prophylactic and/or therapeutic agents simultaneously or sequentially and in a manner such that their respective effects are additive or synergistic.

The term “treating” refers to:

(i) preventing a disease, disorder, or condition from occurring in an animal that may be predisposed to the disease, disorder and/or condition, but has not yet been diagnosed as having it;

(ii) inhibiting the disease, disorder, or condition, i.e., arresting its development; and

(iii) relieving the disease, disorder, or condition, i.e., causing regression of the disease, disorder, and/or condition.

The terms “a” and “P” indicate the specific stereochemical configuration of a substituent at an asymmetric carbon atom in a chemical structure as drawn.

The compounds of the invention may exhibit the phenomenon of tautomerism. While Formula I cannot expressly depict all possible tautomeric forms, it is to be understood that Formula I is intended to represent any tautomeric form of the depicted compound and is not to be limited merely to a specific compound form depicted by the formula drawings. For illustration, and in no way limiting the range of tautomers, the compounds of Formula I may exist as the following:

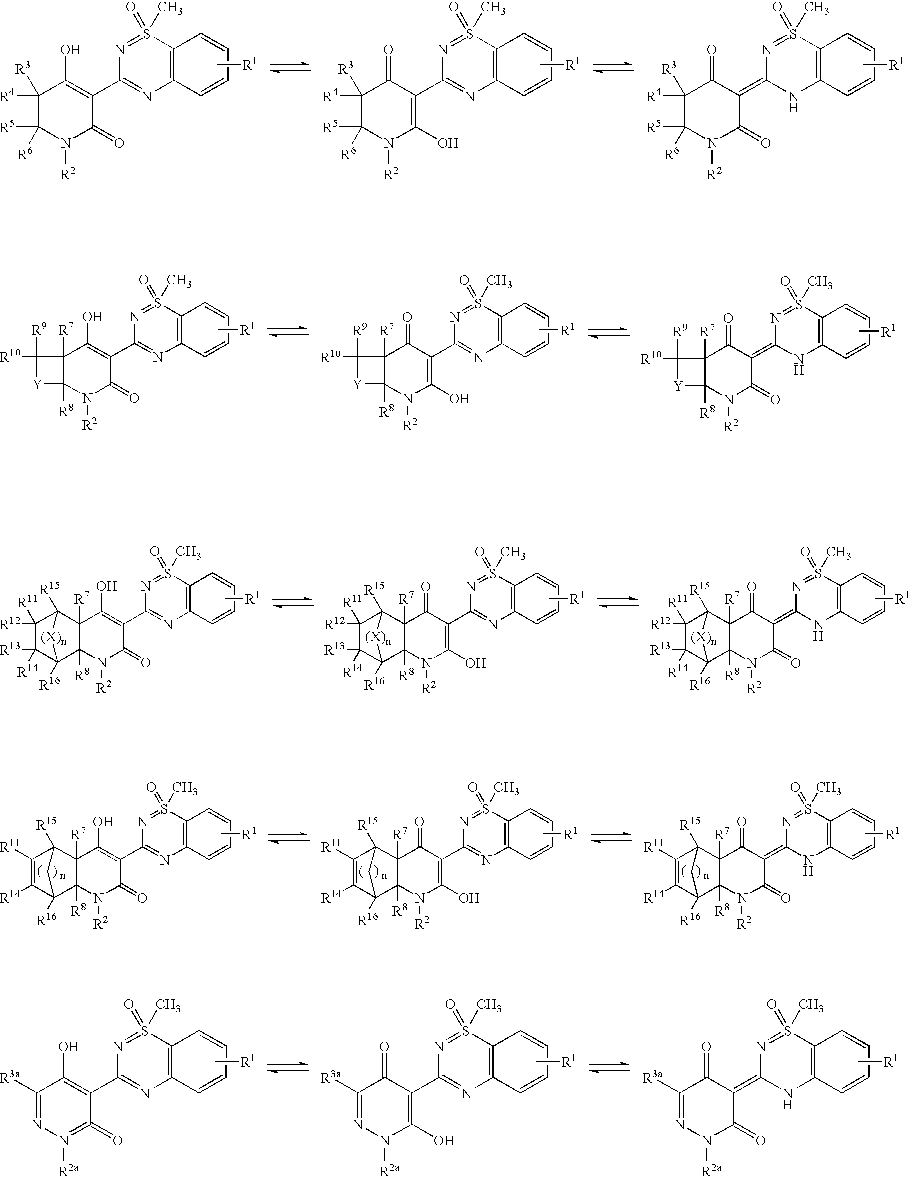

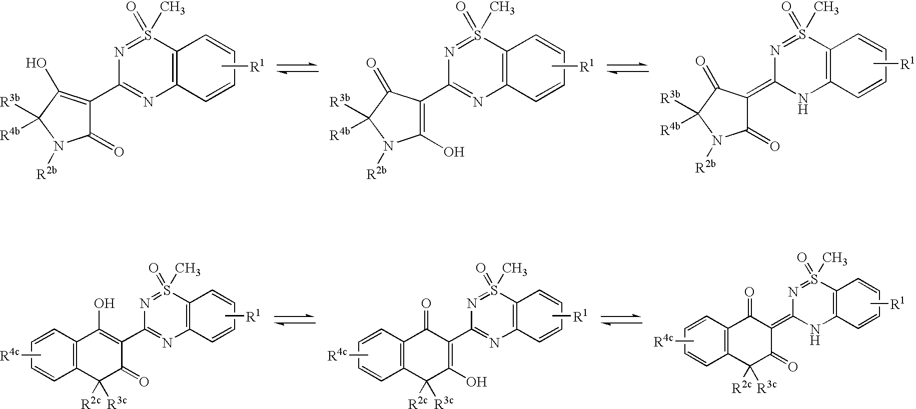

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% free of other enantiomers or diastereomers of the compounds, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the Formula I is intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, the invention includes pharmaceutically acceptable prodrugs, pharmaceutically active metabolites, and pharmaceutically acceptable salts of such compounds and metabolites.

“A pharmaceutically acceptable prodrug” is a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the compounds of Formula I using methods known in the art, such as those described by Burger's Medicinal Chemistry and Drug Chemistry, 1, 172-178, 949-982 (1995). See also Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the Formula I compounds, alter the way in which drugs are distributed in and excreted from the body. However, in some cases, metabolism of a drug is required for therapeutic effect. For example, anticancer drugs of the anti-metabolite class must be converted to their active forms after they have been transported into a cancer cell.

Since most drugs undergo metabolic transformation of some kind, the biochemical reactions that play a role in drug metabolism may be numerous and diverse. The main site of drug metabolism is the liver, although other tissues may also participate.

A feature characteristic of many of these transformations is that the metabolic products, or “metabolites,” are more polar than the parent drugs, although a polar drug does sometime yield a less polar product. Substances with high lipid/water partition coefficients, which pass easily across membranes, also diffuse back readily from tubular urine through the renal tubular cells into the plasma. Thus, such substances tend to have a low renal clearance and a long persistence in the body. If a drug is metabolized to a more polar compound, one with a lower partition coefficient, its tubular reabsorption will be greatly reduced. Moreover, the specific secretory mechanisms for anions and cations in the proximal renal tubules and in the parenchymal liver cells operate upon highly polar substances.

As a specific example, phenacetin (acetophenetidin) and acetanilide are both mild analgesic and antipyretic agents, but are transformed within the body to a more polar and more effective metabolite, p-hydroxyacetanilid (acetaminophen), which is widely used today. When a dose of acetanilide is given to a person, the successive metabolites peak and decay in the plasma sequentially. During the first hour, acetanilide is the principal plasma component. In the second hour, as the acetanilide level falls, the metabolite acetaminophen concentration reaches a peak. Finally, after a few hours, the principal plasma component is a further metabolite that is inert and can be excreted from the body. Thus, the plasma concentrations of one or more metabolites, as well as the drug itself, can be pharmacologically important.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal, polymorphic forms, or co-crystal forms, all of which are intended to be within the scope of the present invention and specified formulas.

### Methods of Treatment and Prevention of Hepatitis C Viral Infections

The present invention provides methods for treating or preventing a hepatitis C virus infection in a patient in need thereof.

The present invention further provides methods for introducing a therapeutically effective amount of the Formula I compound or combination of such compounds into the blood stream of a patient in the treatment and/or prevention of hepatitis C viral infections.

The magnitude of a prophylactic or therapeutic dose of a Formula I compound of the invention or a pharmaceutically acceptable salt, solvate, or hydrate, thereof in the acute or chronic treatment or prevention of an infection will vary, however, with the nature and severity of the infection, and the route by which the active ingredient is administered. The dose, and in some cases the dose frequency, will also vary according to the infection to be treated, the age, body weight, and response of the individual patient. Suitable dosing regimens can be readily selected by those skilled in the art with due consideration of such factors.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of viral infection. These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. Finally, the methods and compositions include the treatment of refractory patients or patients resistant to treatment such as resistance to reverse transcriptase inhibitors, protease inhibitors, etc.

### Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture; alternatively, the dose of the Formula I compound may be formulated in animal models to achieve a circulating plasma concentration range of the compound that corresponds to the concentration required to achieve a fixed magnitude of response. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are responsive to the effects of the Formula I compounds are exposed to the ligand and the magnitude of response is measured by an appropriate technique. The assessment of the Formula I compound is then evaluated with respect to the Formula I compound potency, and the degree of conversion of the Formula I compound prodrug. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a prophylactic or therapeutic dose of a prodrug of a Formula I compound of the invention or a pharmaceutically acceptable salt, solvate, or hydrate thereof in the acute or chronic treatment or prevention of an infection or condition will vary with the nature and severity of the infection, and the route by which the active ingredient is administered. The dose, and perhaps the dose frequency, will also vary according to the infection to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. Also, the dose may differ for various particular Formula I compounds; suitable doses can be predicted on the basis of the aforementioned in vitro measurements and on the basis of animal studies, such that smaller doses will be suitable for those Formula I compounds that show effectiveness at lower concentrations than other Formula I compounds when measured in the systems described or referenced herein. In general, the dose per day is in the range of from about 0.001 to 100 mg/kg, preferably about 1 to 25 mg/kg, more preferably about 5 to 15 mg/kg. For treatment of humans infected by hepatitis C viruses, about 0.1 mg to about 15 g per day is administered in about one to four divisions a day, preferably 100 mg to 12 g per day, more preferably from 100 mg to 8000 mg per day.

Additionally, the recommended daily dose ran can be administered in cycles as single agents or in combination with other therapeutic agents. In one embodiment, the daily dose is administered in a single dose or in equally divided doses. In a related embodiment, the recommended daily dose can be administered once time per week, two times per week, three times per week, four times per week or five times per week.

In one embodiment, the compounds of the invention are administered to provide systemic distribution of the compound within the patient. In a related embodiment, the compounds of the invention are administered to produce a systemic effect in the body.

In another embodiment the compounds of the invention are administered via oral, mucosal (including sublingual, buccal, rectal, nasal, or vaginal), parenteral (including subcutaneous, intramuscular, bolus injection, intraarterial, or intravenous), transdermal, or topical administration. In a specific embodiment the compounds of the invention are administered via mucosal (including sublingual, buccal, rectal, nasal, or vaginal), parenteral (including subcutaneous, intramuscular, bolus injection, intraarterial, or intravenous), transdermal, or topical administration. In a further specific embodiment, the compounds of the invention are administered via oral administration. In a further specific embodiment, the compounds of the invention are not administered via oral administration.

Different therapeutically effective amounts may be applicable for different infections, as will be readily known by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

### Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, antifungal agents, anti-inflammatory agents, antiviral agents, anticancer agents, immunomodulatory agents, α-interferons, β-interferons, ribavirin, alkylating agents, hormones, cytokines, and toll receptor-like modulators. In one embodiment the invention encompasses the administration of an additional therapeutic agent that is HCV specific or demonstrates anti-HCV activity.

The Formula I compounds of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Durice®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefininox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The Formula I compounds of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The Formula I compounds of the invention can be administered or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The Formula I compounds of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofulvin.

The Formula I compounds of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methpredniso lone acetate, methylpredniso lone sodium succinate, mometasone furoate, paramethasone acetate, predniso lone, predniso lone acetate, predniso lone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The Formula I compounds of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, levovirin, viramidine and ribavirin, as well as foscarnet, amantadine, rimantadine, saquinavir, indinavir, amprenavir, lopinavir, ritonavir, the α-interferons; β-interferons; adefovir, clevadine, entecavir, pleconaril.

The Formula I compound of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boehringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), anti-B7 antibodies (e.g., IDEC-114 (IDEC)), CTLA4-immunoglobulin, and toll receptor-like (TLR) modulators. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The Formula I compounds of the invention can be administered or formulated in combination with an agent which inhibits viral enzymes, including but not limited to inhibitors of HCV protease, such as BILN 2061 and inhibitors of NS5b polymerase such as NM107 and its prodrug NM283 (Idenix Pharmaceuticals, Inc., Cambridge, Mass.).

The Formula I compounds of the invention can be administered or formulated in combination with an agent which inhibits HCV polymerase such as those described in Wu, Curr Drug Targets Infect Disord. 2003; 3(3):207-19 or in combination with compounds that inhibit the helicase function of the virus such as those described in Bretner M, et al Nucleosides Nucleotides Nucleic Acids. 2003; 22(5-8): 1531, or with inhibitors of other HCV specific targets such as those described in Zhang X., Drugs, 5(2), 154-8 (2002).

The Formula I compounds of the invention can be administered or formulated in combination with an agent which inhibits viral replication.

The Formula I compounds of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-α, and IFN-γ.

The Formula I compounds of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The Formula I compounds of the invention can be administered or formulated in combination with β-interferons which include, but are not limited to, interferon β-1a, interferon β-1b.

The Formula I compounds of the invention can be administered or formulated in combination with α-interferons which include, but are not limited to, interferon α-1, interferon α-2a (roferon), interferon α-2b, intron, Peg-Intron, Pegasys, consensus interferon (infergen) and albuferon.

The Formula I compounds of the invention can be administered or formulated in combination with an absorption enhancer, particularly those which target the lymphatic system, including, but not limited to sodium glycocholate; sodium caprate; N-lauryl-β-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancers can also be used. Thus, the invention also encompasses a pharmaceutical composition comprising one or more Formula I compounds of the invention and one or more absorption enhancers.

The Formula I compounds of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The compounds of the invention and the other therapeutics agent can act additively or, more preferably, synergistically. In one embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent. In a separate embodiment, a compound of the invention is administered to a patient who has not previously undergone or is not currently undergoing treatment with another therapeutic agent, particularly an antiviral agent.

In one embodiment, the methods of the invention comprise the administration of one or more Formula I compounds of the invention without an additional therapeutic agent.

### Pharmaceutical Compositions and Dosage Forms

Pharmaceutical compositions and single unit dosage forms comprising a Formula I compound of the invention, or a pharmaceutically acceptable salt, or hydrate thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms of the invention typically also comprise one or more pharmaceutically acceptable excipients. Sterile dosage forms are also contemplated.

In an alternative embodiment, pharmaceutical composition encompassed by this embodiment includes a Formula I compound of the invention, or a pharmaceutically acceptable salt, or hydrate thereof, and at least one additional therapeutic agent. Examples of additional therapeutic agents include, but are not limited to, those listed above.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise Formula I compounds of the invention, or a pharmaceutically acceptable salt or hydrate thereof comprise 0.1 mg to 1500 mg per unit to provide doses of about 0.01 to 200 mg/kg per day.

### Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophilized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Typical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985).

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The Formula I compounds of the invention may also be administered directly to the lung by inhalation. For administration by inhalation, a Formula I compound can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a Formula I compound directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a Formula I compound to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a Formula I compound to the lung is a liquid spray device supplied, for example, by Aradigm Corporation. Liquid spray systems use extremely small nozzle holes to aerosolize liquid drug formulations that can then be directly inhaled into the lung.

In one embodiment, a nebulizer device is used to deliver a Formula I compound to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In one embodiment, an electrohydrodynamic (“EHD”) aerosol device is used to deliver Formula I compounds to the lung. EHD aerosol devices use electrical energy to aerosolize liquid drug solutions or suspensions (see, e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the Formula I compounds formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of Formula I compounds will be known to the skilled artisan and are within the scope of the invention.

Liquid drug formulations suitable for use with nebulizers and liquid spray devices and EHD aerosol devices will typically include a Formula I compound with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of the Formula I compound. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. Nos. 5,112,598; Biesalski, 5,556,611, which are herein incorporated by reference) A Formula I compound can also be formulated in rectal or vaginal compositions such as suppositories or retention enemas, e.g., containing conventional suppository bases such as cocoa butter or other glycerides.

In addition to the formulations described previously, a Formula I compound can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver Formula I compounds. Certain organic solvents such as dimethylsulfoxide can also be employed, although usually at the cost of greater toxicity. A Formula I compound can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 1983, 23, 61; see also Levy et al., Science, 1985, 228, 190; During et al., Ann. Neurol., 1989, 25, 351; Howard et al., J. Neurosurg., 71, 105 (1989). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g. Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

The invention provides a pharmaceutical pack or kit comprising one or more containers comprising a Formula I compound useful for the treatment or prevention of a Hepatitis C virus infection. In other embodiments, the invention provides a pharmaceutical pack or kit comprising one or more containers comprising a Formula I compound useful for the treatment or prevention of a Hepatitis C virus infection and one or more containers comprising an additional therapeutic agent, including but not limited to those listed above, in particular an antiviral agent, an interferon, an agent which inhibits viral enzymes, or an agent which inhibits viral replication, preferably the additional therapeutic agent is HCV specific or demonstrates anti-HCV activity.

The invention also provides a pharmaceutical pack or kit comprising one or more containers comprising one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The inventive agents may be prepared using the reaction routes and synthesis schemes as described below, employing the general techniques known in the art using starting materials that are readily available. The synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### Preparation of Compounds

In the synthetic schemes described below, unless otherwise indicated all temperatures are set forth in degrees Celsius and all parts and percentages are by weight.

Reagents were purchased from commercial suppliers such as Aldrich Chemical Company or Lancaster Synthesis Ltd. and were used without further purification unless otherwise indicated. All solvents were purchased from commercial suppliers such as Aldrich, EMD Chemicals or Fisher and used as received.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried.

The reactions were assayed by TLC and/or analyzed by LC-MS and terminated as judged by the consumption of starting material. Analytical thin layer chromatography (TLC) was performed on glass-plates precoated with silica gel 60 F₂₅₄ 0.25 mm plates (EMD Chemicals), and visualized with UV light (254 nm) and/or iodine on silica gel and/or heating with TLC stains such as ethanolic phosphomolybdic acid, ninhydrin solution, potassium permanganate solution or ceric sulfate solution. Preparative thin layer chromatography (prepTLC) was performed on glass-plates precoated with silica gel 60 F₂₅₄ 0.5 mm plates (20×20 cm, from Thomson Instrument Company) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. Column chromatography was completed under positive pressure using Merck silica gel 60, 230-400 mesh or 50-200 mesh neutral alumina, ISCO Flash-chromatography using prepacked RediSep silica gel columns, or Analogix flash column chromatography using prepacked SuperFlash silica gel columns. Hydrogenolysis was done at the pressure indicated in the examples or at ambient pressure.

¹H-NMR spectra and ¹³C-NMR were recorded on a Varian Mercury-VX400 instrument operating at 400 MHz. NMR spectra were obtained as CDCl₃ solutions (reported in ppm), using chloroform as the reference standard (7.26 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), bs (broad singlet), dd (doublet of doublets), dt (doublet of triplets). Coupling constants, when given, are reported in Hertz (Hz).

Infrared (IR) spectra were recorded on an ATR FT-IR Spectrometer as neat oils or solids, and when given are reported in wave numbers (cm⁻¹). Mass spectra reported are (+)-ES or APCI (+) LC/MS conducted by the Analytical Chemistry Department of Anadys Pharmaceuticals, Inc. Elemental analyses were conducted by the Atlantic Microlab, Inc. in Norcross, Ga. Melting points (mp) were determined on an open capillary apparatus, and are uncorrected.

The described synthetic pathways and experimental procedures may utilize many common chemical abbreviations, including 2,2-DMP (2,2-dimethoxypropane), Ac (acetyl), ACN (acetonitrile), Bn (benzyl), Boc (tert-butoxycarbonyl), Boc₂O (di-tert-butyl dicarbonate), Bz (benzoyl), NCS(N-chloro-succinamide), DBU (1,8-diazabicyclo[5,4,0]undec-7-ene), DCC(N,N′-dicyclohexylcarbodiimide), DCE (1,2-dichloroethane), DCM (dichloromethane), DEAD (diethylazodicarboxylate), DIEA (diisopropylethylamine), DMA (N,N-dimethylacetamide), DMAP (4-(N,N-dimethylamino)pyridine), DMF (N,N-dimethylformamide), DMSO (dimethyl sulfoxide), EDC (1-(3-dimethylaminopropyl)-3-ethylcarbodiimide hydrochloride), Et (ethyl), EtOAc (ethyl acetate), EtOH (ethanol), HATU (O-(7-azabenzotriazol-1-yl)-1,1,3,3-tetramethyluronium hexafluorophosphate), HBTU (O-benzotriazol-1-yl-N,N,N′,N′-tetramethyluronium hexafluorophosphate), HF (hydrogen fluoride), HOAc (acetic acid), HOBT (1-hydroxybenzotriazole hydrate), HPLC (high pressure liquid chromatography), IPA (isopropyl alcohol), KHMDS (potassium bis(trimethylsilyl)amide), KN(TMS)₂ (potassium bis(trimethylsilyl)amide), KO^{t}Bu (potassium tert-butoxide), LDA (lithium diisopropylamine), MCPBA (3-chloroperbenzoic acid), Me (methyl), MeCN (acetonitrile), MeOH (methanol), NaH (sodium hydride), NaN(TMS)₂ (sodium bis(trimethylsilyl)amide), NaOAc (sodium acetate), NaOEt (sodium ethoxide), Phe (phenylalanine), PPTS (pyridinium p-toluenesulfonate), PS (polymer supported), Py (pyridine), pyBOP (benzotriazol-1-yloxy)tripyrrolidinophosphonium hexafluorophosphate), TEA (triethylamine), TFA (trifluoroacetic acid), TFAA (trifluoroacetic anhydride), THF (tetrahydro furan), TLC (thin layer chromatography), Tol (toluoyl), Val (valine), and the like. Scheme 1 provides a general procedure that may be used to prepare compounds of Formula I.

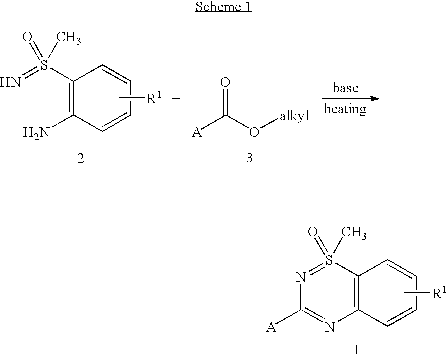

In this general procedure, the sulfoximine 2 can react with ester 3 in the presence of a base and heating condition to form the desired products of Formula I.

### EXAMPLE 1-1

### Scheme 1a provides a specific procedure that was used to prepare 5-(4-fluoro-benzyl)-8-hydroxy-7-(1-methyl-1 oxo-1λ⁶-benzo[1,2,4]thiadiazin-3-yl)-5-aza-spiro[2.5]oct-7-en-6-one (9) of Formula I

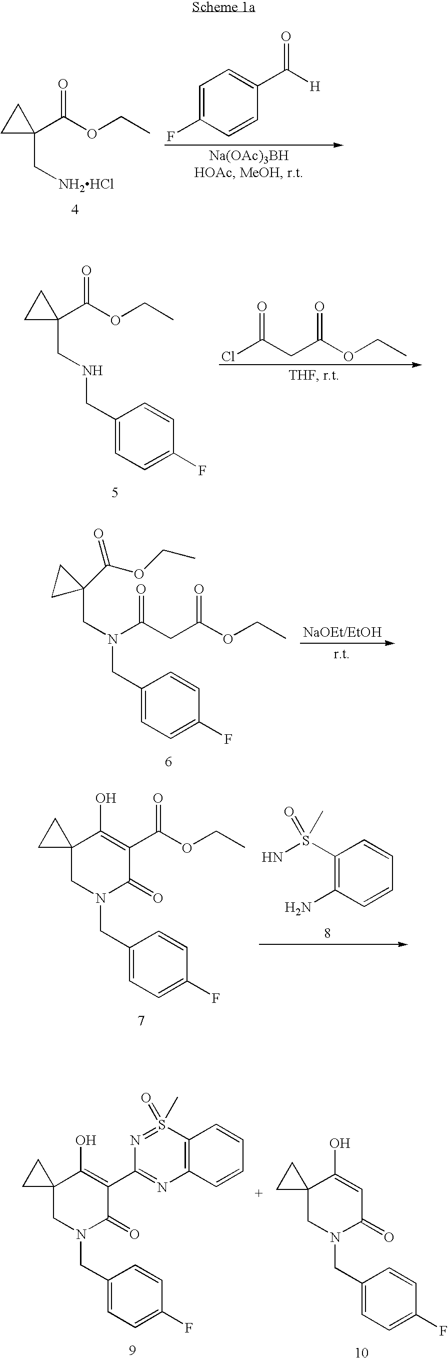

### Step 1: Synthesis of 1-[(4-fluoro-benzylamino)-methyl]-cyclopropanecarboxylic acid ethyl ester (5)

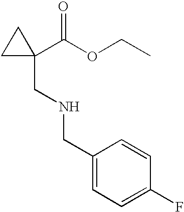

The above amino-acid ethyl ester hydrochloride (4) (625.8 mg, 4.37 mmol) was dissolved in MeOH (20 mL), the para-F-benzaldehyde (460 μL, 4.37 mmol) was added at room temperature followed by HOAc (830 μL) and Na(OAc)₃BH (2.3 g, 10.93 mmol) portion-wise as solid at 0° C. (ice-H₂O bath). The reaction mixture was stirred at room temperature overnight. The LC-MS analysis confirmed the completion of the reaction. Saturated aqueous NaHCO₃ solution (30 mL) was added to neutralize the reaction (pH=8). The desired product was extracted out with EtOAc (50 mL×3) and the combined organic layers were washed with brine (25 mL), dried over anhydrous Na₂SO₄, filtered and concentrated under reduced pressure at room temperature to give the crude product (5) (870.1 mg) in 79.2% yield. LC-MS (m/e): 252.1 [M+1]⁺ (exact ms: 251.13). This crude product was directly used in next step.

### Step 2: Synthesis of 1-{[(4-fluoro-benzyl)-(3-oxo-hexanoyl)-amino]-methyl}-cyclopropanecarboxylic acid ethyl ester (6)

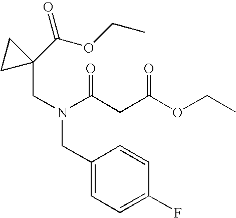

Compound 5 (274.4 mg, 1.09 mmol) was dissolved in THF (5 mL), chlorocarbonyl-acetic acid ethyl ester (0.22 mL, 1.64 mmol) was added and the resulted mixture was stirred at room temperature overnight. LC-MS analysis showed only 50% conversion. Additional chlorocarbonyl-acetic acid ethyl ester (0.22 mL) was added and the resulted mixture was stirred at room temperature overnight. LC-MS analysis confirmed the reaction was close to completion. H₂O (10 mL) was added and the product was extracted out with EtOAc (15 mL×3). The organic layer was further washed with saturated aqueous NaHCO₃ (10 mL) and brine (10 mL), dried over anhydrous Na₂SO₄, filtered, concentrated to give 591.7 mg of crude product (6) as orange oil which was directly used in next step without further purification. LC-MS (m/e): 366.2 [M+1]⁺ (exact ms: 365.2).

### Step 3: Synthesis of 5-(4-fluoro-benzyl)-8-hydroxy-6-oxo-5-aza-spiro[2.5]oct-7-ene-7-carboxylic acid ethyl ester (7)

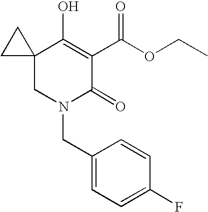

Compound 6 (102.8 mg, 0.28 mmol) was dissolved in EtOH (0.6 mL). NaOEt (21 wt % in EtOH, 0.15 mL) was added and the resulted mixture was shaken at room temperature for 1 hour. LC-MS analysis confirmed the completion of the reaction. HCl (1.0 M aqueous) was added to adjust the pH to 2. H₂O (11n L) and brine (1 mL) was added and the product was extracted out with EtOAc (51n L×3), dried over anhydrous Na₂SO₄, filtered, concentrated and dried under reduced pressure to give the desired product (7) (66.6 mg. 74.6% yield) with HPLC purity of 90% by ELSD. LC-MS (m/e): 320.1 [M+1]⁺ (exact ms: 319.1)

### Step 4: Synthesis of 5-(4-fluoro-benzyl)-8-hydroxy-7-(1-methyl-1-oxo-1λ⁶-benzo[1,2,4]thiadiazin-3-yl)-5-aza-spiro[2.5]oct-7-en-6-one (9)

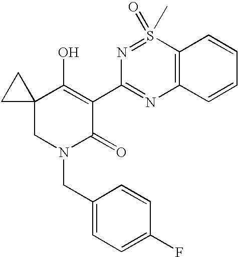

Compound 7 (29.6 mg, 82.3 μmol) and compound 8 (21 mg, 123 μmol) were mixed and dissolved in pyridine (0.5 mL) in a sealed tube. The mixture was heated in a pre-heated oil bath at 110° C. for 2 hours then at 120° C. for 2 hours. LC-MS confirmed the completion of the reaction. The reaction mixture was concentrated at reduced pressure with heating to give a crude product (9) containing the de-carboxylated side product (10) of the starting material (7) based on the LC-MS analysis. Reverse-phase HPLC purification with 20-100% of ACN in H₂O with 0.05% TFA gave the pure desired product (9) (4.6 mg) with 100% HPLC purity by ELSD. LC-MS (m/e): 426.3 [M+1]⁺ (exact ms: 425.1).

### EXAMPLE 1-2

### Scheme 1b describes the synthesis of 3-(4-fluoro-benzyl)-6-hydroxy-5-(1-methyl-1-oxo-1λ⁶-benzo[1,2,4]thiadiazin-3-yl)-3-aza-tricyclo[6.2.1.0^{2,7}]undec-5-en-4-one (14) of Formula I

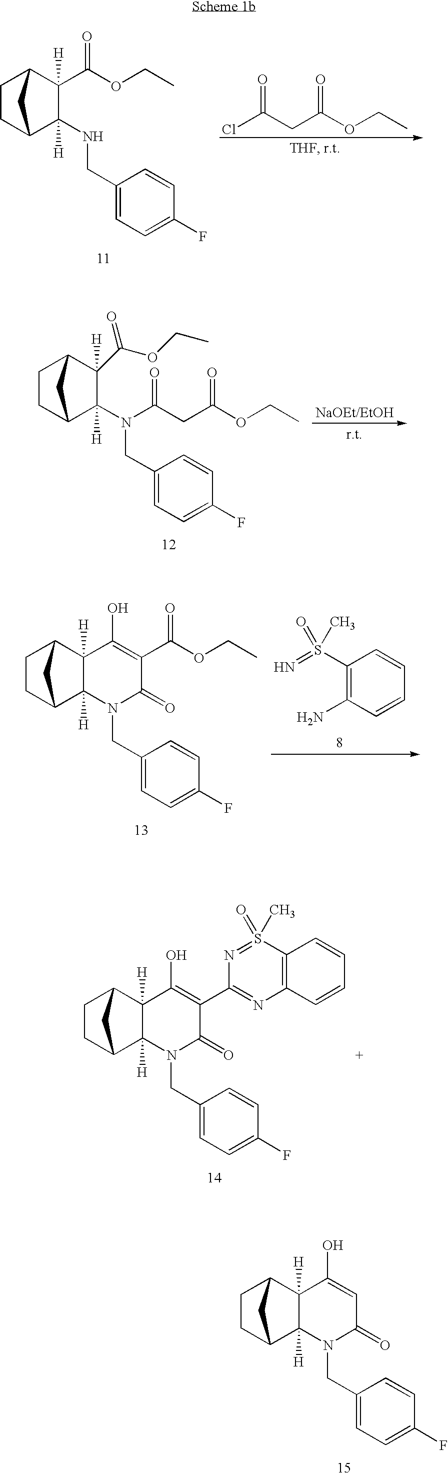

### Step 1: Synthesis of 3-[(2-ethoxycarbonyl-acetyl)-(4-fluorobenzyl)-amino]-bicyclo[2.2.1]heptane-2-carboxylic acid ethyl ester (12)

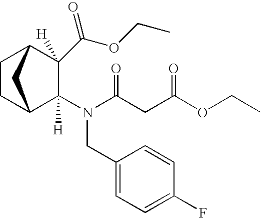

THF (10 mL) and dioxane (2 mL) were mixed with compound 11 (98% ee) (583 mg, 2 mmol) to form slurry. Chlorocarbonyl-acetic acid ethyl ester (0.4 mL, 3 mmol) was added and the resulted mixture was stirred at room temperature overnight. LC-MS analysis showed only less than 50% conversion. DMA (1 mL) was added to solubilize the reaction mixture and additional chlorocarbonyl-acetic acid ethyl ester (0.8 mL) was added and the resulted mixture was stirred at room temperature overnight. LC-MS analysis confirmed the completion of the reaction. The mixture was poured onto H₂O (10 mL), extracted with EtOAc (15 mL×3) and the organic layer was washed with brine (10 mL), dried over anhydrous MgSO₄, filtered, concentrated to give 1.85 g of crude desired product (12) which was directly used in next step without further purification. LC-MS (m/e): 406.4 [M+1]⁺ (exact ms: 405.2).

### Step 2: Synthesis of 3-(4-fluoro-benzyl)-6-hydroxy-4-oxo-3-aza-tricyclo[6.2.1.0^{2,7}]undec-5-ene-5-carboxylic acid ethyl ester (13)

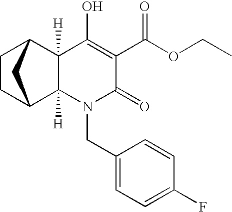

Compound 12 (104.4 mg, 0.26 mmol) was dissolved in EtOH (0.5 mL) and NaOEt (21 wt % in ethanol, 0.14 mL) was added and the resulted mixture was shaken at room temperature for 1 hour. LC-MS analysis showed incompletion of the reaction. Additional NaOEt (21 wt % in ethanol, 0.14 mL) was added and the resulted mixture was shaken at room temperature overnight. LC-MS analysis confirmed the completion of the reaction. Aqueous HCl (1.0 M, 1 mL) was added followed by H₂O (1 mL) and brine (1 mL), extracted with EtOAc (5 mL×3), dried with anhydrous Na₂SO₄, filtered, concentrated under reduced pressure to give the crude desired product (13) (59.1 mg) in 63.3% yield which was directly used in next step. LC-MS (ES⁺) (m/e): 360.2 (exact ms: 359).

### Step 3: Synthesis of 3-(4-fluoro-benzyl)-6-hydroxy-5-(1-methyl-1-oxo-1λ⁶-benzo[1,2,4]thiadiazin-3-yl)-3-aza-tricyclo[6.2.1.0^{2,7}]undec-5-en-4-one (14)

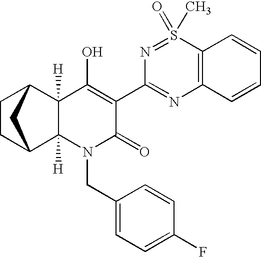

Compound 13 (29.6 mg, 82.3 μmol) was dissolved in pyridine (0.5 mL). Compound 8 (21 mg, 123 μmol) was added and the resulted mixture was heated in a sealed tube at 110° C. for 2 hours and then at 120° C. for 2 hours. LC-MS analysis confirmed the completion of the reaction. The solvent was removed by heating at 70° C. under reduced pressure. Reverse-phase HPLC purification (20-100% CH₃CN in H₂O with 0.05% TFA) gave the desired product (14) (6.2 mg) which contained the de-carboxylated side product (15) of the starting material 13 with mass of 287.3 based on LC-MS analysis. LC-MS (ES⁺) (m/e): 466.3 [M+1]⁺ (exact ms: 465.2).

### EXAMPLE 1-3

### Scheme 1a Provides a Specific Procedure that was Used to Prepare Compound 17 of Formula I

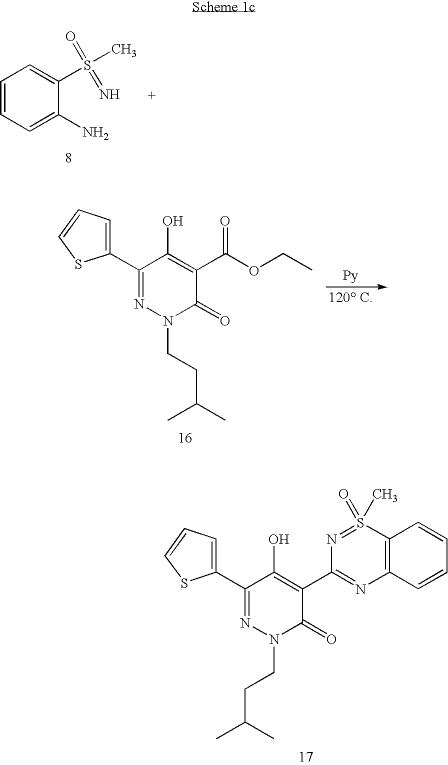

In this specific example, compound 8 (59 mg, 0.35 mmol) was mixed with pyridazinone 16 (141 mg, 0.42 mmol) and dissolved in 10 mL of pyridine. The reaction mixture was heated at 120° C. for 3 hours, concentrated in vacuo. The flash column chromatography purification (MeOH/CH₂Cl₂) gives the pure desired product of 5-hydroxy-2-(3-methyl-butyl)-4-(1-methyl-1-oxo-1λ⁶-benzo[1,2,4]thiadiazin-3-yl)-6-thiophen-2-yl-2H-pyridazin-3-one (17) (48.4 mg, 31% yield). LC-MS (ES⁺): m/e=443.3 [M+1]⁺ (Exact Mass: 442.1). ¹H NMR (400 MHz, CDCl₃) δ: 1.01 (6H, d, J=6.2 Hz), 1.27 (1H, s), 1.70-1.80 (2H, m), 3.77 (3H, s), 4.13-4.18 (2H, m), 7.10 (1H, t, J=4.3 Hz), 7.36-7.37 (1H, m), 7.47 (2H, apparent triplet, J=8.1 Hz), 7.76 (1H, t, J=7.8 Hz), 7.80 (1H, d, J=7.8 Hz), 7.99 (1H, d, J=3.9 Hz).

### EXAMPLE 1-4

### Scheme 1d Describes Another Procedure, Which was Used to Synthesize Compound 20, 21 and 22 of Formula I

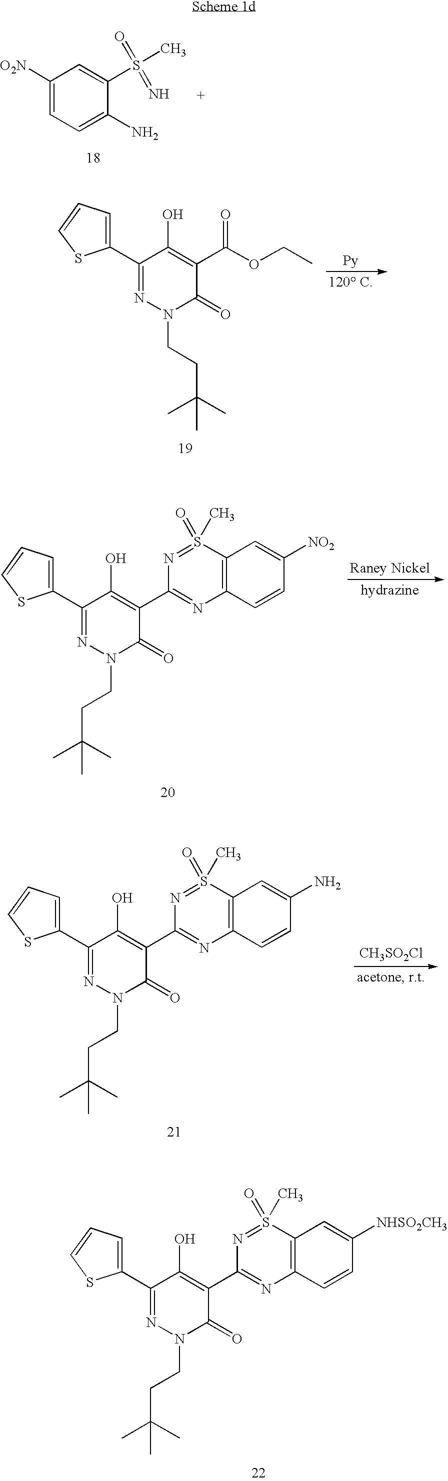

### Synthesis of 2-(3,3-dimethyl-butyl)-5-hydroxy-4-(1-methyl-7-nitro-1-oxo-1λ⁶-benzo[1,2,4]thiadiazin-3-yl)-6-thiophen-2-yl-2H-pyridazin-3-one (20)

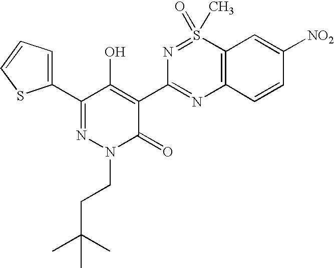

Sulfoximine aniline (18) (71 mg, 0.33 mmol) and pyridazinone (19) (77.2 mg, 0.22 mmol) were mixed and dissolved in pyridine (1 mL). The reaction mixture was heated at 120° C. with stirring under N₂ atmosphere for 9 h. LC-MS spectrum indicated that there was some uncyclized amide intermediate left. DBU (150 μL) was added and the reaction mixture was stirred at 120° C. overnight. The majority of the pyridine solvent was removed under reduced pressure at 70° C. using rotavaporer. Water (2 mL) was added and the product was extracted out with CHCl₃ (4 mL×3). The combined organic layers were concentrated in vacuo and purified by flash column chromatography to afford the desired product (20) (84.8 mg) containing some unknown impurity. This crude product was directly used in the next step without further purification. LC-MS (ES⁺): m/e: 502.2 [M+1]⁺ (exact ms: 501.1).

### Synthesis of 4-(7-amino-1-methyl-1-oxo-1λ⁶-benzo[1,2,4]thiadiazin-3-yl)-2-(3,3-dimethyl-butyl)-5-hydroxy-6-thiophen-2-yl-2H-pyridazin-3-one (21)

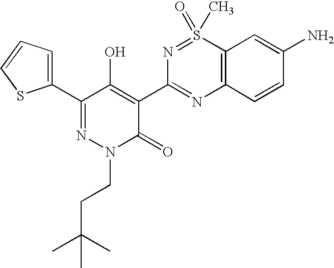

Compound 20 (84.8 mg) was dissolved in THF (1.5 mL) and MeOH (1.5 mL). Raney Nickel (50% slurry in H₂O) (0.5 mL) was added with stirring at room temperature followed immediately by hydrazine (0.2 mL). The mixture was stirred at room temperature for 1 h. LC-MS spectrum confirmed the completion of the reaction. The solid was filtered off, washed with MeOH and the filtrate was concentrated in vacuo to give the crude product (21) (77.7 mg) which was directly used in next step without further purification.

### Synthesis of N-{3-[2-(3,3-dimethyl-butyl)-5-hydroxy-3-oxo-6-thiophen-2-yl-2,3-dihydro-pyridazin-4-yl]-1-methyl-1-oxo-1l6-benzo[1,2,4]thiadiazin-7-yl}-methanesulfonamide (22)

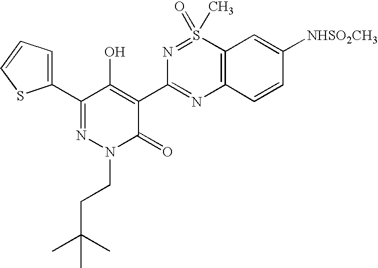

Compound 21 (77.7 mg) was dissolved in acetone (3 mL). Pyridine (260 μL) was added followed by methyl sulfonyl chloride (175 μL). The reaction mixture was stirred at room temperature overnight. LC-MS spectrum confirmed the completion of the reaction. HCl (1.0 M, 1.8 mL) was added to adjust pH to about 3. H₂O (2 mL) and brine (2 mL) were then added and the product was extracted out by CHCl₃ (10 mL×3). The combined organic layers were concentrated in vacuo. The HPLC purification of the crude product (with gradient of 40-95% ACN in H₂O) gave the pure desired product (22, 7.86 mg, 6.5% isolated yield for 3 steps) as a yellow solid. ¹H NMR (400 MHz, DMSO-d₆) δ: 0.96 (s, 9H), 1.60-1.64 (m, 2H), 3.15 (s, 3H), 4.01 (s, 3H), 4.02-4.04 (m, 2H), 7.08-7.10 (m, 1H), 7.53-7.54 (m, 1H), 7.64-7.65 (m, 2H), 7.86-7.87 (m, 1H), 7.90-7.91 (m, 1H), 10.28 (s, 1H), LC-MS (ESIF): m/e=550.2 [M+1]⁺ (exact mass: 549.12).

### Chiral Separation of Compound 22

The above compound 22 is a mixture of 2 enantiomers which were separated by normal phase-HPLC (high pressure liquid chromatography) purification using a chiral column (ChiralPak AD 250×21.2 mm ID) and a gradient of 0.2% TFA in EtOH using isocratic run for 46 minutes at 4.0 mL/min detected at 360 nm (UV detector). The compound (22) concentration used for the chiral separation was 2 mg/mL with 2 mg per injection. The separated 2 enantiomers (23 and 24) are listed below. Compound 23 obtained after chiral HPLC purification has HPLC purity of 99.8% with 99.5% ee. Compound 24 thus obtained has HPLC purity of 97.2% with 100% ee. The chiral analysis of the enantiomer excess (ee) was done with a chiral column (ChiralPak AD 250×4.6 mm ID) and a gradient of 0.2% TFA in EtOH using isocratic run for 20 minutes at 0.4 mL/min, detected at 360 nm (UV detector). A solution of 0.5 mg/mL (5 μL) of the compound (23 or 24) in 5-10% DMSO in EtOH was used for the chiral analysis.

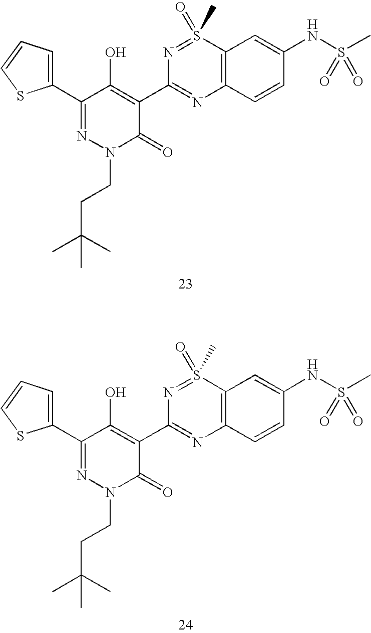

### EXAMPLE 1-5

### Scheme 1e Describes a Similar Procedure, which was used to Synthesize Compound 26, 27 and 28 of Formula I

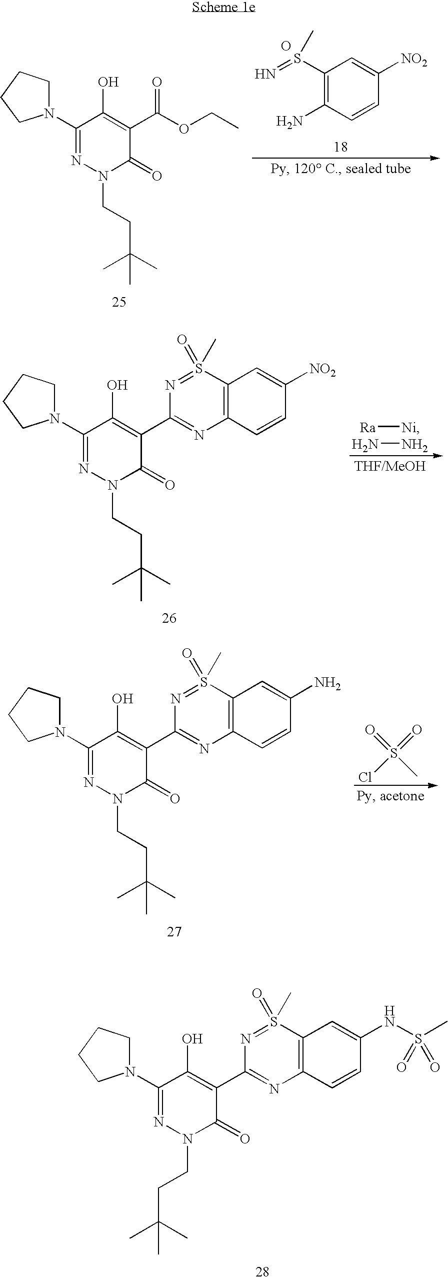

### Synthesis of 2-(3,3-dimethyl-butyl)-5-hydroxy-4-(1-methyl-7-nitro-1-oxo-1λ⁶-benzo[1,2,4]thiadiazin-3-yl)-6-pyrrolidin-1-yl-2H-pyridazin-3-one (26)

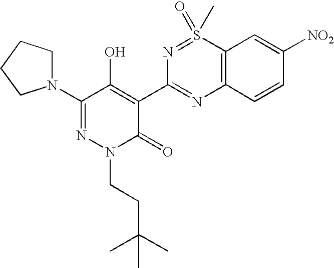

To a solution of 2-(3,3-dimethyl-butyl)-5-hydroxy-3-oxo-6-pyrrolidin-1-yl-2,3-dihydro-pyridazine-4-carboxylic acid ethyl ester (25) (0.1 g, 0.31 mmol) in pyridine (1.5 mL), (18) (94.5 mg, 0.44 mmol) was added and the mixture was heated in a sealed tube at 120° C. for 9 h. After cooling, the mixture was diluted with ethyl acetate (100 mL) and then washed in a separatory funnel with 1N HCl (40 mL) via extraction. The organic layer was then concentrated to dryness under reduced pressure and then the product was crystallized out from a mixture solvents (8 mL) of 70% ethyl acetate in hexanes to afford the desired product of 2-(3,3-dimethyl-butyl)-5-hydroxy-4-(1-methyl-7-nitro-1-oxo-1λ⁶-benzo[1,2,4]thiadiazin-3-yl)-6-pyrrolidin-1-yl-2H-pyridazin-3-one (26) as a yellow solid (47 mg, 31% yield). ¹H NMR (400 MHz, DMSO-d₆) δ: 0.93 (s, 9H), 1.54-1.58 (m, 2H), 1.78-1.81 (m, 4H), 3.45-3.49 (m, 4H), 3.81-3.85 (m, 2H), 4.12 (s, 3H), 7.71 (d, 1H, J=9.4 Hz), 8.50 (dd, 1H, J₁=9.4 Hz, J₂=4.7 Hz), 9.24 (d, 1H, J=2.2 Hz), LC-MS (ESI⁺): m/e=489.3 [M+1]⁺ (exact mass: 488.18).

### Synthesis of 4-(7-amino-1-methyl-1-oxo-1λ⁶-benzo[1,2,4]thiadiazin-3-yl)-2-(3,3-dimethyl-butyl)-5-hydroxy-6-pyrrolidin-1-yl-2H-pyridazin-3-one (27)

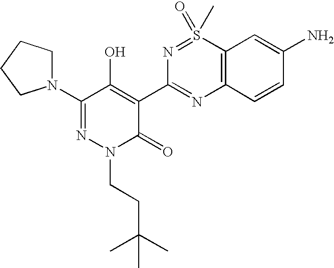

To a solution of 2-(3,3-dimethyl-butyl)-5-hydroxy-4-(1-methyl-7-nitro-1-oxo-1λ⁶-benzo[1,2,4]thiadiazin-3-yl)-6-pyrrolidin-1-yl-2H-pyridazin-3-one (26) (47 mg, 0.096 mmol) dissolved in THF (1 mL) and MeOH (1 mL), we added Raney-Nickel in 50% water immediately followed by hydrazine (0.113 mL). The reaction mixture was stirred at room temperature for 15 minutes. The solid was removed by filtering through Celite, washed with methanol, and the filtrate was concentrated to dryness under reduced pressure to afford the crude product (85 mg, quantitative yield) of 4-(7-Amino-1-methyl-1-oxo-1λ⁶-benzo[1,2,4]thiadiazin-3-yl)-2-(3,3-dimethyl-butyl)-5-hydroxy-6-pyrrolidin-1-yl-2H-pyridazin-3-one (27), LC-MS (ESI⁺): m/e=459.3 [M+1]⁺ (exact mass: 458.21).

### Synthesis of N-{3-[2-(3,3-dimethyl-butyl)-5-hydroxy-3-oxo-6-pyrrolidin-1-yl-2,3-dihydro-pyridazin-4-yl]-1-methyl-1-oxo-1λ⁶-benzo[1,2,4]thiadiazin-7-yl}-methanesulfonamide (28)

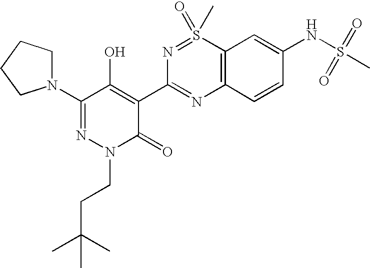

To a solution of 4-(7-amino-1-methyl-1-oxo-1λ⁶-benzo[1,2,4]thiadiazin-3-yl)-2-(3,3-dimethyl-butyl)-5-hydroxy-6-pyrrolidin-1-yl-2H-pyridazin-3-one (27), (44 mg, 0.096 mmol) in acetone (1.6 mL), pyridine (0.156 mL, 1.9 mmol) was added followed by dropwise addition of methane sulfonylchloride (0.111 mL, 1.4 mmol). The reaction mixture was stirred at 25° C. for 3 h. The reaction mixture was diluted with ethyl acetate (50 mL) and washed with 0.5 N HCl (50 mL), and brine (50 mL) via extraction. The organic layer was dried over anhydrous MgSO₄ and concentrated to dryness under reduced pressure. The resulting crude was resuspended in ethyl acetate (2 mL) at which point product crystallizes out of solution, which was collected by filtration and further dried under high vacuum to afford N-{3-[2-(3,3-dimethyl-butyl)-5-hydroxy-3-oxo-6-pyrrolidin-1-yl-2,3-dihydro-pyridazin-4-yl]-1-methyl-1-oxo-1λ⁶-benzo[1,2,4]thiadiazin-7-yl}-methanesulfonamide (28) (9.32 mg, 17.8% isolated yield) as a yellow solid. ¹H NMR (400 MHz, DMSO-d₆) δ: 0.92 (s, 9H), 1.53-1.56 (m, 2H), 1.78 (bs, 4H), 3.12 (s, 3H), 3.45 (bs, 4H), 3.79-3.83 (m, 2H), 3.94 (s, 3H), 7.56-7.58 (m, 2H), 7.82 (s, 1H), 10.19 (s, 1H), LC-MS (ESI⁺): m/e=537.4 [M+1]⁺ (exact mass: 536.19).

### EXAMPLE 1-6

### Scheme 1f Provides a Synthetic Method that was Used to Prepare Compounds 30, 31 and 32 of Formula I

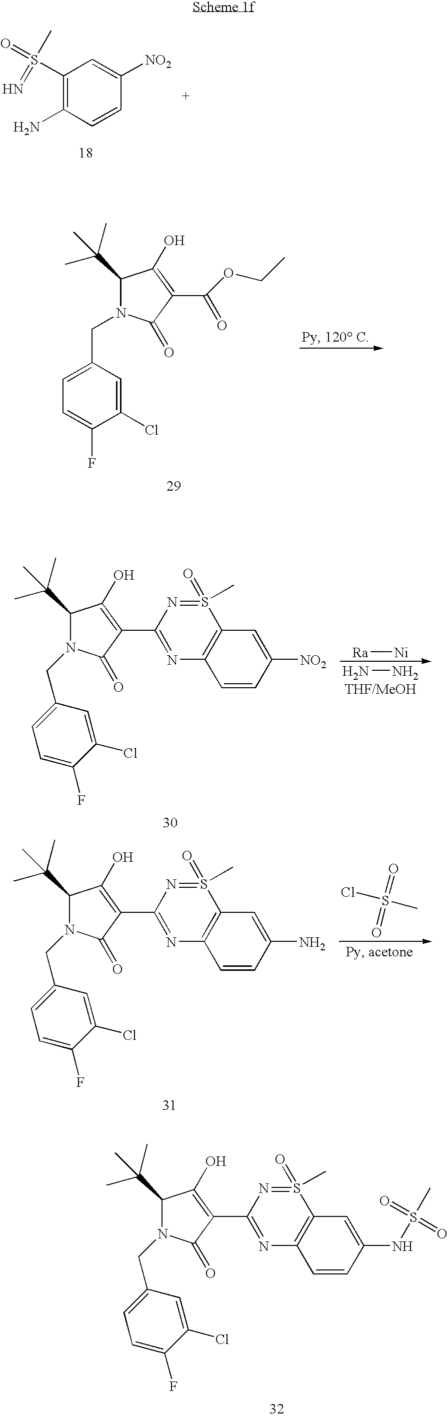

### Synthesis of 5-tert-butyl-1-(3-chloro-4-fluoro-benzyl)-4-hydroxy-3-(1-methyl-7-nitro-1-oxo-1λ⁶-benzo[1,2,4]thiadiazin-3-yl)-1,5-dihydro-pyrrol-2-one (30)

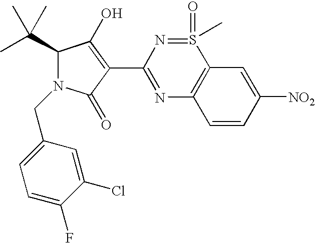

To a solution of 5-tert-butyl-1-(3-chloro-4-fluoro-benzyl)-4-hydroxy-2-oxo-2,5-dihydro-1H-pyrrole-3-carboxylic acid ethyl ester (29) (0.187 g, 0.5 mmol)) in pyridine (2.5 mL, 0.2 M), 2-amino-5-nitro-benzenesulfoximine 18 (0.154 mg, 0.71 mmol) was added. The mixture was heated in a sealed tube at 120° C. for 3 h, cooled to room temperature, diluted with ethyl acetate (100 mL) and then washed in a separatory funnel with 1N HCl (100 mL). The layers were separated. Aqueous layer was extracted one more time with EtOAc (100 mL), and the combined organic layer was concentrated to dryness under reduced pressure. The residue was purified by column chromatography on silica gel to afford 5-tert-butyl-1-(3-chloro-4-fluoro-benzyl)-4-hydroxy-3-(1-methyl-7-nitro-1-oxo-1λ⁶-benzo[1,2,4]thiadiazin-3-yl)-1,5-dihydro-pyrrol-2-one (30) (0.118 g, 45%). LC-MS (ESI⁺): m/e 521.3 [M+1]⁺ (exact ms: 520.1).

### Synthesis of 3-(7-amino-1-methyl-1-oxo-1λ⁶-benzo[1,2,4]thiadiazin-3-yl)-5-tert-butyl-1-(3-chloro-4-fluoro-benzyl)-4-hydroxy-1,5-dihydro-pyrrol-2-one (31)

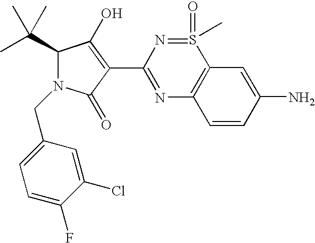

To a solution of 5-tert-butyl-1-(3-chloro-4-fluoro-benzyl)-4-hydroxy-3-(1-methyl-7-nitro-1-oxo-1λ⁶-benzo[1,2,4]thiadiazin-3-yl)-1,5-dihydro-pyrrol-2-one (30) (0.118 g, 0.22 mmol) in THF (1.1 mL) and MeOH (1.1 mL), added Raney-Nickel (50% slurry in water) and stirred for 5 minutes, after which time hydrazine (0.266 mL) was added in dropwise manner. The reaction was stirred at room temperature for 15 minutes. The solid was filtered off through Celite, washed with methanol, and the filtrate was concentrated to dryness under reduced pressure to afford 3-(7-amino-1-methyl-1-oxo-1λ⁶-benzo[1,2,4]thiadiazin-3-yl)-5-tert-butyl-1-(3-chloro-4-fluoro-benzyl)-4-hydroxy-1,5-dihydro-pyrrol-2-one (31) (0.046 g, 39% yield) as a dark brown solid. This crude product was directly used in next step without further purification. LC-MS (ESI⁺): m/e 491.3 [M+1]⁺ (exact ms: 490.12).

### Synthesis of N-{3-[5-tert-butyl-1-(3-chloro-4-fluoro-benzyl)-4-hydroxy-2-oxo-2,5-dihydro-1H-pyrrol-3-yl]-1-methyl-1-oxo-1λ⁶-benzo[1,2,4]thiadiazin-7-yl}-methanesulfonamide (32)

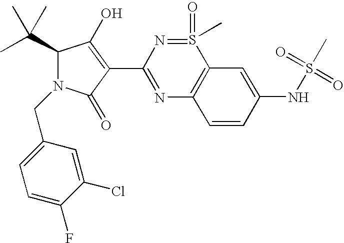

To a solution of 3-(7-amino-1-methyl-1-oxo-1λ⁶-benzo[1,2,4]thiadiazin-3-yl)-5-tert-butyl-1-(3-chloro-4-fluoro-benzyl)-4-hydroxy-1,5-dihydro-pyrrol-2-one (31) (0.046 g, 0.093 mmol) in acetone (1.5 mL) added pyridine (0.153 mL, 1.87 mmol) followed by dropwise addition of methane sulfonylchloride (0.108 mL, 1.39 mmol). The reaction mixture was stirred at room temperature for 2.5 h, then diluted with ethyl acetate (50 mL), washed with 0.5 N HCl (50 mL) and brine (50 mL) via extraction. The separated organic layer was dried over MgSO₄ and concentrated to dryness under reduced pressure. The crude product thus obtained was further purified by prep-TLC plate coated with a thin layer of silica gel to afford N-{3-[5-tert-butyl-1-(3-chloro-4-fluoro-benzyl)-4-hydroxy-2-oxo-2,5-dihydro-1H-pyrrol-3-yl]-1-methyl-1-oxo-1λ⁶-benzo[1,2,4]thiadiazin-7-yl}-methanesulfonamide (32) (3.9 mg, 7.4% yield) as a solid. ¹H NMR (400 MHz, CDCl₃) δ: 1.08 (s, 9H), 3.08 (s, 3H), 3.23-3.33 (m, 1H), 3.59 (s, 3H), 4.30 (d, 1H, J=15.6 Hz), 5.17 (d, 1H, J=16.3 Hz), 7.05-7.09 (m, 2H), 7.22-7.24 (m, 2H), 7.57-7.67 (m, 1H), 7.75-7.75 (m, 1H). LC-MS (ESI⁺): m/e 569.2 [M+1]⁺ (exact ms: 568.1).

### EXAMPLE 1-7

### Scheme 1g Describes a Procedure, which was Used to Synthesize Compound 34, 35 and 36 of Formula I

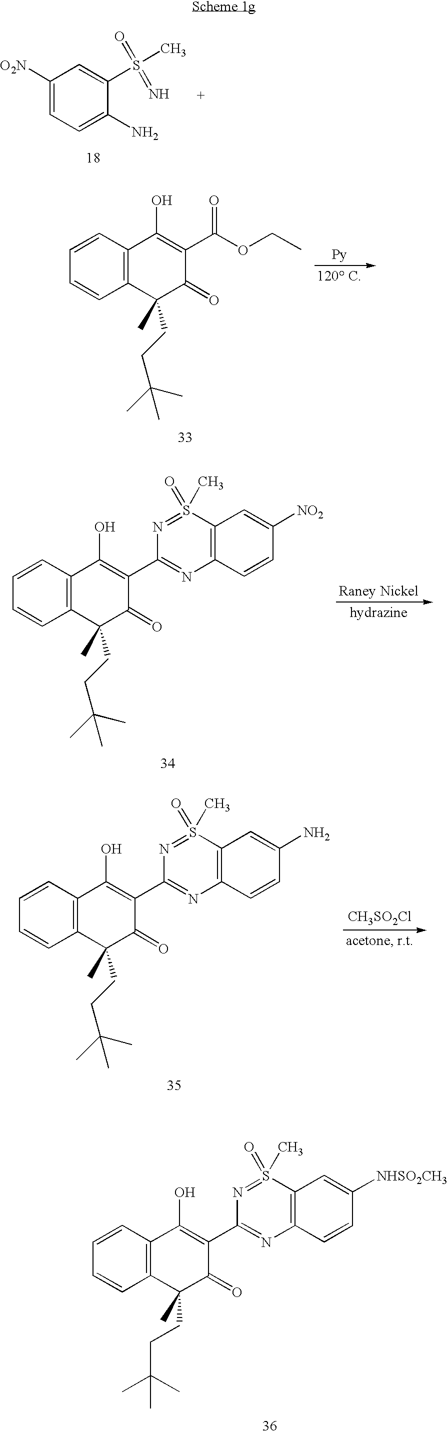

### Synthesis of 1-(3,3-dimethyl-butyl)-4-hydroxy-1-methyl-3-(1-methyl-7-nitro-1-oxo-1λ⁶-benzo[1,2,4]thiadiazin-3-yl)-1H-naphthalen-2-one (34)

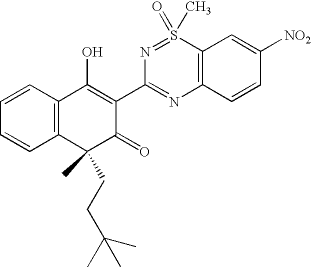

Sulfoximine aniline (18) (304.5 mg, 1.41 mmol) and compound 33 (310.5 mg, 0.94 mmol) was mixed and dissolved in pyridine (4 mL). The reaction mixture was heated at 120° C. with stirring under N₂ atmosphere for 6.5 h. The majority of the pyridine solvent was removed under reduced pressure at 70° C. using a rotavaporer. The residue was purified by flash column chromatography on silica gel to afford the desired product (34) (354.4 mg, 78.3% crude yield). This crude product was used directly in the next step without further purification. LC-MS (ES⁺): m/e: 482.3 [M+1]⁺ (exact ms: 481.2).

### Synthesis of 3-(7-amino-1-methyl-1-oxo-1λ⁶-benzo[1,2,4]thiadiazin-3-yl)-1-(3,3-dimethyl-butyl)-4-hydroxy-1-methyl-1H-naphthalen-2-one (35)

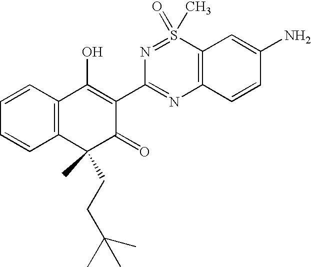

Compound 34 (327.8 mg, 0.68 mmol) was dissolved in THF (5 mL) and MeOH (5 mL). Raney Nickel (50% slurry in H₂O) (1.23 mL) was added with stirring at room temperature immediately followed by hydrazine (NH₂NH₂) (0.493 mL). The mixture was stirred at room temperature for 35 minutes. LC-MS spectrum confirmed the completion of the reaction with quantitative conversion. The solid was filtered off, washed with MeOH and the filtrate was concentrated in vacuo to give the crude product (35) which was directly used in next step without further purification. LC-MS (ESI⁺): m/e=452.3 [M+1]⁺ (Exact Mass: 451.2).

### Synthesis of N-{3-[4-(3,3-dimethyl-butyl)-1-hydroxy-4-methyl-3-oxo-3,4-dihydro-naphthalen-2-yl]-1-methyl-1-oxo-1λ⁶-benzo[1,2,4]thiadiazin-7-yl}-methanesulfonamide (36)

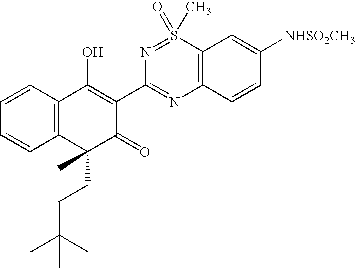

Compound 35 (0.68 mmol) was dissolved in acetone (10 mL). Pyridine (1.1 mL) was added followed by methyl sulfonyl chloride (530 μL). The reaction mixture was stirred at room temperature for 10.5 h. LC-MS spectrum confirmed the completion of the reaction. HCl (1.0 M) was added to adjust pH to about 2-3. Brine (1.5 mL) and H₂O (1.5 mL) were then added and the product was extracted with CHCl₃ (5 mL×2) and EtOAc (5 mL). The combined organic layers were concentrated in vacuo to give the crude product. The HPLC purification of the crude product (with gradient of 40-95% ACN in H₂O) gave the pure desired product (36) as a mixture of the diastereomers (3:1) (46.1 mg, 12.8% isolated yield for 2 steps). LC-MS (ESI⁺): m/e=530.3 [M+1]⁺ (Exact Mass: 529.2). ¹H NMR (400 MHz, CDCl₃) δ: 8.24 (d, 1H, J=8 Hz), 7.96 (s, 1H), 7.78-7.84 (m, 1H), 7.58 (t, 1H, J=7.6 Hz), 7.38-7.44 (m, 3H), 3.79 (s, br, 3H), 3.11 (s, br, 3H), 2.25-2.37 (m, 1H), 1.82-1.92 (m, 1H), 1.59 (s, 41% of 3H), 1.58 (s, 59% of 3H), 0.86-0.95 (m, 1H), 0.761 (s, 58% of 9H), 0.75 (s, 42% of 9H), 0.55-0.66 (m, 1H).

### Chiral Separation of Compound 36

The above compound 36 is a mixture of 2 diastereomers which were separated by normal phase-HPLC (high pressure liquid chromatography) purification using a chiral column (ChiralPak AD 250×21.2 mm ID) and a gradient of 40% of n-heptane and 60% of a solvent mixture of n-heptane/MeOH/EtOH (=1:1:1) with 0.3% TFA using isocratic run for 35 minutes at 8.0 mL/min detected at 360 nm (UV detector) with 8-9 mg per injection. The separated 2 diastereomers (37 and 38) are listed below. Compound 37 obtained after chiral HPLC purification has HPLC purity of 99.5% with 98.9% de. Compound 38 thus obtained has HPLC purity of 99.9% with 100% de. The chiral analysis of the diastereomer excess (de) was done using a chiral column (ChiralPak AD 250×4.6 mm ID) and same gradient as that used in the separation using isocratic run for 18 minutes at 0.7 mL/min, detected at 360 nm (UV detector). A solution of 2 mg/mL of either compound 37 or 38 in 2% DMSO in mobile phase solvents was used for the chiral analysis.

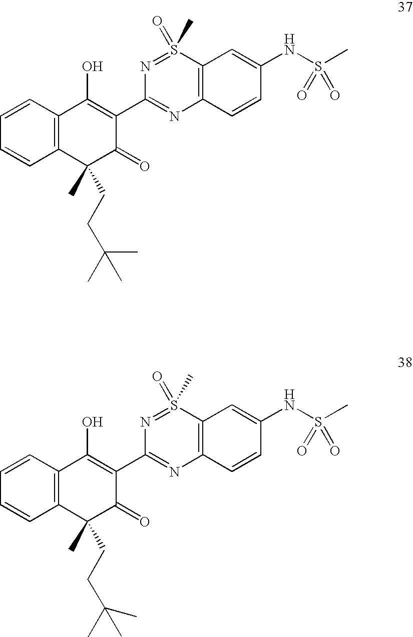

Scheme 2a provides a general procedure that can be used to prepare compounds 42 of Formula I.

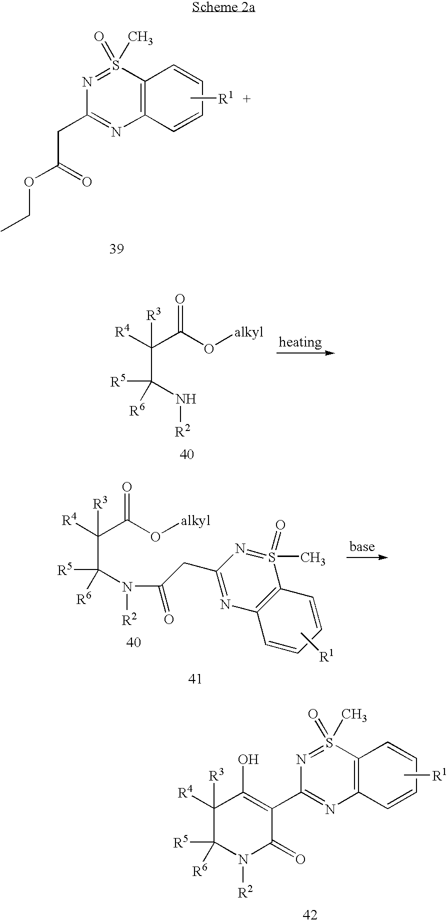

Sulfoximide ester 39 and 1-amino-ester 40 can be mixed and dissolved in a solvent selected from toluene, dioxane, NMP and pyridine in a sealed tube at temperature range from 100-180° C. to form the amide 41 which can be cyclized in the presence of a base selected from pyridine, DBU, Et₃N and NaOEt to form the desired product 42.

Similarly compounds 44, 46 and 48 can be prepared as shown in Scheme 2b, Scheme 2c and Scheme 2d, respectively.

Scheme 2b provides a general procedure that can be used to prepare compounds 44 of Formula I.

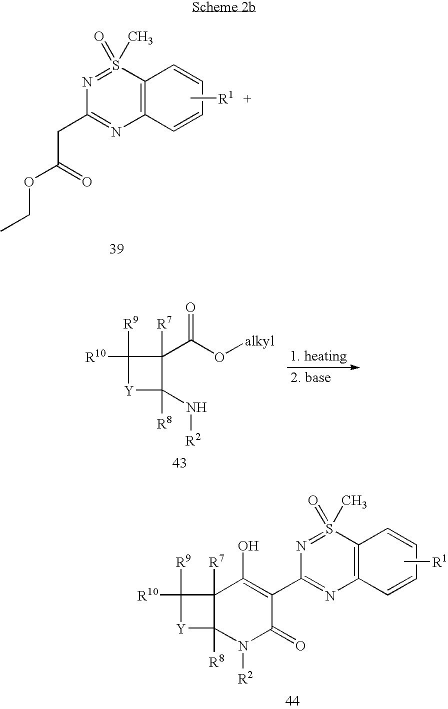

Scheme 2c provides a general procedure that can be used to prepare compounds 46 of Formula I.

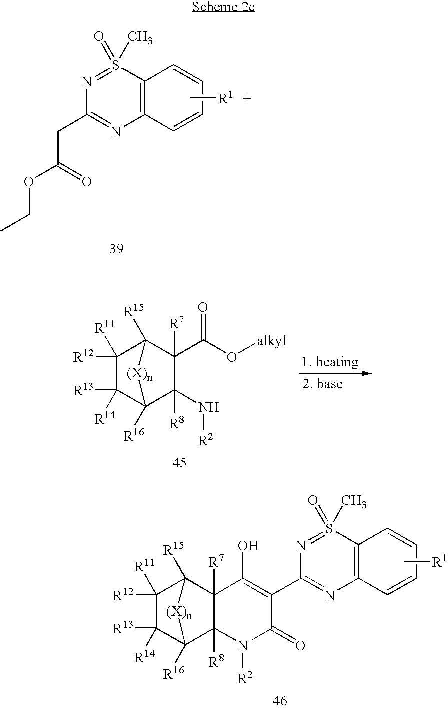

Scheme 2d provides a general procedure that can be used to prepare compounds 48 of Formula I.

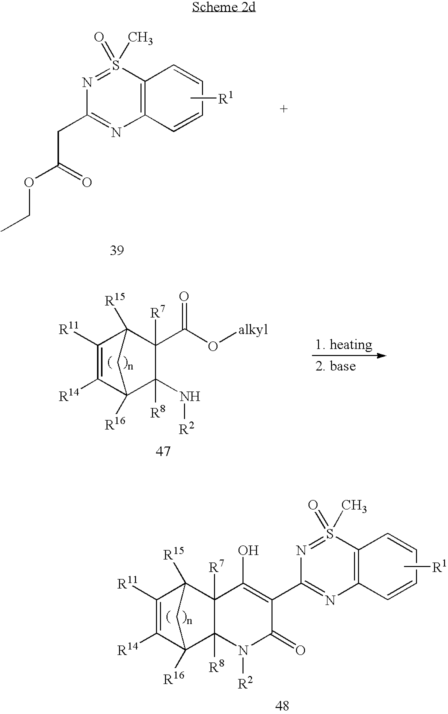

### EXAMPLE 2-1

### Scheme 2e Describes the Synthesis of N-{3-[3-(4-fluoro-benzyl)-6-hydroxy-4-oxo-3-aza-tricyclo[6.2.1.0^{2,7}]undec-5-en-5-yl]-1-methyl-1-oxo-1λ⁶-benzo[1,2,4]thiadiazin-7-yl}-methanesulfonamide (54)

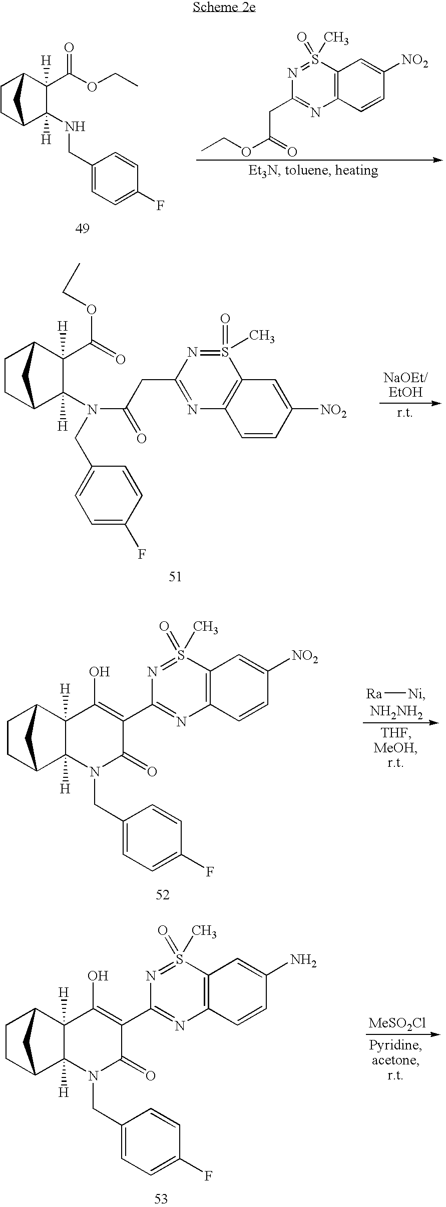

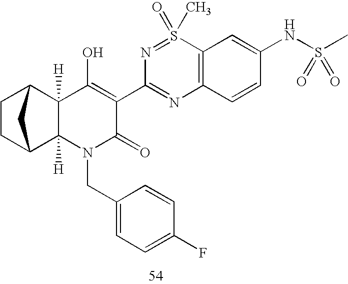

### Step 1: Synthesis of 3-{(4-fluoro-benzyl)-[2-(1-methyl-7-nitro-1-oxo-1λ⁶-benzo[1,2,4]thiadiazin-3-yl)-acetyl]-amino}-bicyclo[2.2.1]heptane-2-carboxylic acid ethyl ester (51)

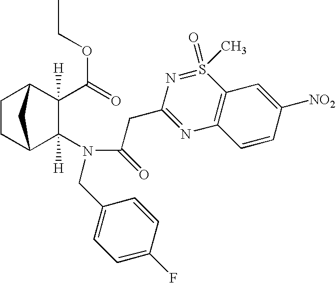

Compound 49 (98% ee) (226 mg, 0.776 mmol) was mixed with compound 50 (241.5 mg, about 50% pure based on LC-MS by ELSD, 0.39 mmol). Et₃N (0.6 mL, 3.1 mmol), toluene (11 mL) and DMA (2 mL) were added and the resulted mixture was heated in a sealed tube at 116° C. for 4 days. LC-MS analysis showed incompletion of reaction. The reaction mixture was concentrated and dried under reduced pressure first, then re-dissolved in toluene (10 mL) and the resulted solution was heated in a sealed tube at 150° C. overnight. LC-MS analysis confirmed the completion of the reaction based on the disappearance of the starting material of 50. The solvent was removed under reduced pressure to give the crude product of 51 that contained the further cyclized product 52 and large excess starting material of 49. This crude product was directly used in next step without further purification. LC-MS (ES⁺) (m/e): 557.3 [M+1]⁺ (exact ms: 556).

### Step 2: Synthesis of 3-(4-fluoro-benzyl)-6-hydroxy-5-(1-methyl-7-nitro-1-oxo-1λ⁶-benzo[1,2,4]thiadiazin-3-yl)-3-aza-tricyclo[6.2.1.0^{2,7}]undec-5-en-4-one (52)

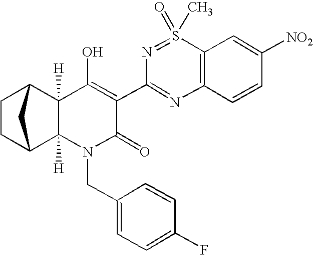

Ethanol (5 mL) was mixed with above full amount of compound 51 (≦0.39 mmol) to form slurry. NaOEt (21 wt % in EtOH, 0.4 mL) was added and the resulted mixture was stirred at room temperature for 1.6 hours. LC-MS analysis confirmed the completion of the reaction. Aqueous HCl (1.0 M, 3 mL) and H₂O (7 mL) were added, extracted with EtOAc (15 mL×3), dried with Na₂SO₄, filtered and concentrated under reduced pressure to give the crude desired product 52 that contained starting material 49. This crude was directly used in next step without further purification. LC-MS (ES⁺) (m/e): 511.4 (exact ms: 510.1).

### Step 3: Synthesis of 5-(7-amino-1-methyl-1-oxo-1λ⁶ benzo[1,2,4]thiadiazin-3-yl)-3-(4-fluoro-benzyl)-6-hydroxy-3-aza-tricyclo[6.2.1.0^{2,7}]undec-5-en-4-one (53)

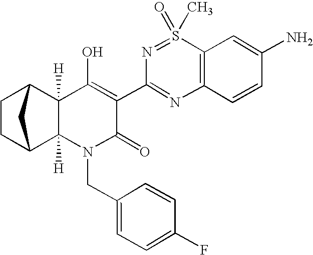

The above full amount of compound 52 (≦0.39 mmol) was dissolved in THF (5 mL) and MeOH (5 mL). Raney Nickel (50% slurry in water, 1.4 mL) was added at 0° C. (ice-H₂O bath) immediately followed by hydrazine (563 μL neat) with stirring at 0° C. The resulted mixture was stirred at room temperature for 20 minutes. LC-MS analysis showed the incompletion of the reaction. Additional Raney Nickel (50% slurry in water, 2 mL) and hydrazine (0.8 mL) were added and the resulted mixture was stirred at room temperature for 1.5 hour. LC-MS analysis confirmed the completion of the reaction. The solid was filtered off and washed with MeOH (5 mL) and THF (51n L) and the filtrate was concentrated under reduced pressure and dried on high vacuum overnight to remove residual hydrazine to give the desired product (53) that contained starting material 49. LC-MS (ES⁺) (m/e): 481.3 [M+1]⁺ (exact ms: 480).

### Step 4: Synthesis of N-{3-[3-(4-fluoro-benzyl)-6-hydroxy-4-oxo-3-aza-tricyclo[6.2.1.0^{2,7}]undec-5-en-5-yl]-1-methyl-1-oxo-1λ⁶-benzo[1,2,4]thiadiazin-7-yl}-methanesulfonamide (54)

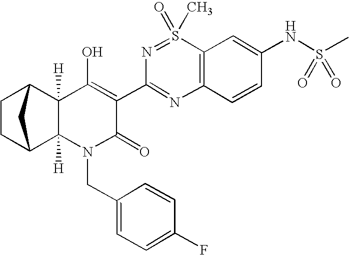

The full amount of above compound 53 (≦0.39 mmol) was dissolved in acetone (6 mL). Pyridine (0.2 mL) and methyl sulfonyl chloride (120 μL) were added and the resulted mixture was stirred at room temperature for 5 hours. LC-MS analysis showed the incompletion of the reaction with some starting material remaining. Additional acetone (4 mL), pyridine (1 mL) and methyl sulfonyl chloride (484 μL) were added and the resulted mixture was stirred at room temperature for 2 hours. LC-MS analysis showed formation of di-sulfonylated side product in addition to the desired product. Water (5 mL), brine (5 mL) and aqueous HCl (1.0 M, 1.5 mL) were added, extracted with EtOAc (20 mL×3) and the organic layer was dried with MgSO₄, filtered, concentrated under reduced pressure to give a crude product. This crude product was purified by normal phase chromatography on silica gel with a gradient of 0-10% MeOH in DCM to give a mixture (16.6 mg) containing the desired product 54, starting material 49 and other unidentified impurity. This mixture was further purified by reverse-phase HPLC purification (15-100% CH₃CN in H₂O with 0.05% TFA) to give the pure desired product 54 (1.0 mg) in 0.5% yield for 4 steps with >95% HPLC purity by ELSD. LC-MS (ES⁺) (m/e): 559.2 [M+1]⁺ (exact ms: 558.14).

### EXAMPLE 2-2

### Scheme 2f Describes a Similar Procedure, Which was Used to Synthesize Compounds 61, 62 and 63 of Formula I

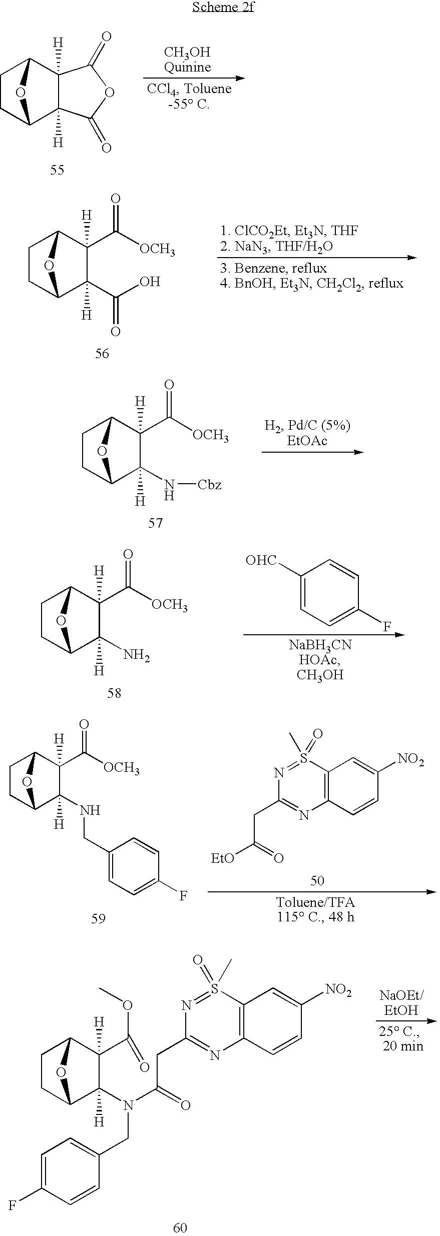

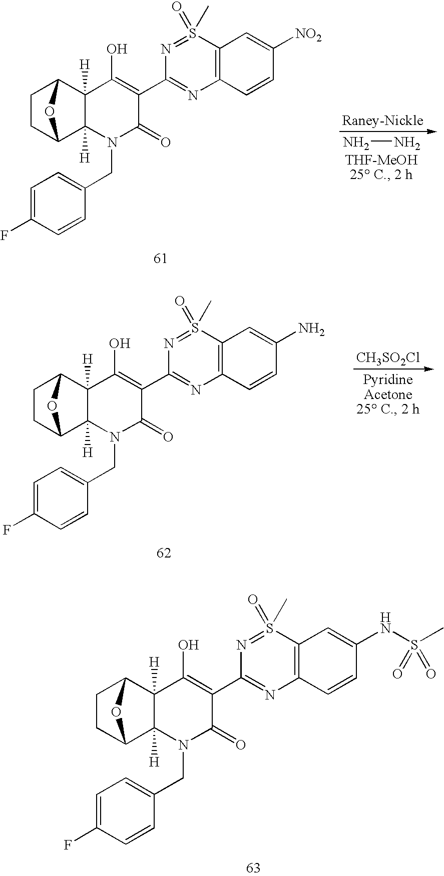

### Synthesis of (1S,2R,3S,4R)-3-(methoxycarbonyl)-7-oxabicyclo[2.2.1]heptane-2-carboxylic acid (56)

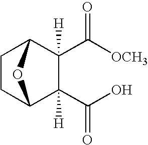

4,10-Dioxa-tricyclo[5.2.1.0^{2,6}]decane-3,5-dione (55) (5.10 g, 30.3 mmol) was dissolved in a 1:1 mixture of toluene and carbon tetrachloride (600 mL). The mixture was cooled to −55° C. under a nitrogen atmosphere, and then quinine (10.54 g, 32.5 mmol) was added. Methanol (3.59 mL, 90 mmol) in toluene-carbon tetrachloride (1:1, 30 mL) was slowly added via an addition funnel. The suspension was stirred at −55° C. for 60 h, and then allowed to warm to room temperature. The mixture was concentrated in vacuo and the residue was dissolved in ethyl acetate (400 mL), washed with aqueous 1.0 M hydrochloric acid solution (2×300 mL) and brine, dried over magnesium sulfate and filtered. The filtrate was concentrated in vacuo to afford the desired product, (1S,2R,3S,4R)-3-(methoxycarbonyl)-7-oxabicyclo[2.2.1]heptane-2-carboxylic acid (56) (2.46 g, 12.3 mmol, 41% yield) as a clear oil. ¹H NMR (400 MHz, DMSO-d₆) δ: 1.49-1.53 (4H, m), 2.99 (2H, s), 3.50 (3H, s), 4.66 (2H, m), 12.15 (1H, s).

### Synthesis of methyl (1R,2S,3R,4S)-3-{[(benzyloxy)carbonyl]amino}-7-oxabicyclo[2.2.1]heptane-2-carboxylate (57)

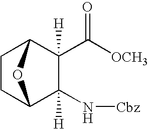

(1S,2R,3S,4R)-3-(Methoxycarbonyl)-7-oxabicyclo[2.2.1]heptane-2-carboxylic acid (56) (2.46 g, 12.3 mmol) was dissolved in anhydrous tetrahydrofuran (35 mL) and cooled to −10° C. under a nitrogen atmosphere. Triethylamine (5.13 mL, 36.9 mmol) was added followed by the dropwise addition of ethyl chloroformate (2.35 mL, 24.6 mmol) with vigorous stirring. Immediate precipitation was observed. The mixture was stirred at −10° C. for 1 h. Sodium azide (2.40 g, 36.9 mmol) was dissolved in water (17 mL) and added to the reaction mixture at −10° C. The mixture was stirred at −10° C. for 15 minutes, and then was allowed to warm to room temperature and stirred for 2 h. The mixture was poured into water (100 mL) and extracted with ethyl acetate. The combined organic layers were washed with saturated aqueous sodium bicarbonate solution and brine, dried over magnesium sulfate and filtered. The filtrate was concentrated in vacuo to afford the acyl azide intermediate as a clear oil. The oil was dissolved in anhydrous benzene (80 mL) and refluxed for 2 h under a nitrogen atmosphere. The solution was allowed to cool to room temperature, and concentrated in vacuo to afford a yellow oil. The oil was dissolved in dichloromethane (45 mL). Triethylamine (3.46 mL, 24.6 mmol) and benzyl alcohol (1.27 mL, 12.3 mmol) were added sequentially. The resulting mixture was refluxed for 16 h under a nitrogen atmosphere. The mixture was allowed to cool to room temperature, concentrated in vacuo and the residue was purified by flash column chromatography (Teledyne Isco RediSep Flash Column 120 g; 0-50% ethyl acetate in hexane as gradient) to afford the desired product, methyl (1R,2S,3R,4S)-3-{[(benzyloxy)carbonyl]amino}-7-oxabicyclo[2.2.1]heptane-2-carboxylate (57) (2.23 g, 7.30 mmol, 59% yield) as a clear oil. ¹H NMR (400 MHz, CDCl₃) δ: 1.51 (2H, m), 1.72 (1H, m), 1.79 (1H, m), 2.97 (1H, d, J=8.4 Hz), 3.56 (3H, s), 4.33 (1H, m), 4.37 (d, 1H, J=5.6 Hz), 4.78 (1H, d, J=4.4 Hz), 5.10 (2H, m), 5.42 (1H, d, J=10.0 Hz), 7.35 (5H, m); LC-MS (ES⁺) (m/e): 306.5 [M+H⁺] (exact ms: 305.1).

### Synthesis of methyl (1R,2S,3R,4S)-3-amino-7-oxabicyclo[2.2.1]heptane-2-carboxylate (58)

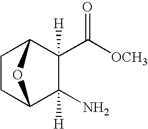

To a solution of methyl (1R,2S,3R,4S)-3-{[(benzyloxy)carbonyl]amino}-7-oxabicyclo[2.2.1]heptane-2-carboxylate (57) (2.23 g, 7.30 mmol) in ethyl acetate (60 mL), 5% palladium on carbon (0.5 g, 22% by weight) was added. The flask was degassed and backfilled with hydrogen gas with a balloon. The mixture was stirred at room temperature for 16 h, passed through a plug of celite and rinsed with ethyl acetate. The filtrate was concentrated in vacuo to afford the desired product, methyl (1R,2S,3R,4S)-3-amino-7-oxabicyclo[2.2.1]heptane-2-carboxylate (58) (1.0 g, 5.84 mmol, 80% yield) as a clear oil. ¹H NMR (400 MHz, CDCl₃) δ: 1.43 (2H, m), 1.67 (1H, m), 1.76 (1H, m), 2.82 (1H, d, J=7.6 Hz), 3.41 (1H, d, J=7.6 Hz), 3.73 (3H, s), 4.28 (1H, d, J=6.0 Hz), 4.81 (1H, d, J=4.8 Hz).

### Synthesis of methyl (1R,2S,3R,4S)-3-[(4-fluorobenzyl)amino]-7-oxabicyclo[2.2.1]heptane-2-carboxylate (59)

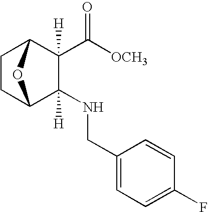

4-Fluoro-benzaldehyde (Aldrich) (0.62 mL, 5.85 mmol) was added to a solution of methyl (1R,2S,3R,4S)-3-amino-7-oxabicyclo[2.2.1]heptane-2-carboxylate (58) (1.02 g, 5.85 mmol) in anhydrous CH₃OH (20 mL) at 25° C. under a nitrogen atmosphere. After stirring for 10 minutes, glacial acetic acid (Fisher Scientific) (0.8 mL) and sodium cyanoborohydride (Aldrich) (920 mg, 14.6 mmol) were added sequentially, and the resulting mixture was stirred at 25° C. for 18 h. The reaction mixture was poured into saturated NaHCO₃ solution and extracted with EtOAc. The combined organic layers were washed with brine, dried over Na₂SO₄ and concentrated in vacuo. The residue was dried in high vacuum to afford desired product, methyl (1R,2S,3R,4S)-3-[(4-fluorobenzyl)amino]-7-oxabicyclo[2.2.1]heptane-2-carboxylate (59) (1.40 g, 86% yield) as a colorless oil. ¹H NMR (400 MHz, CDCl₃) δ: 7.26 (m, 2H), 6.99 (m, 2H), 4.73 (d, 1H, J=4.8 Hz), 4.46 (d, 1H, J=5.2 Hz), 3.84 (d, 1H, J=13.6 Hz), 3.73 (s, 3H), 3.68 (d, 1H, J=13.6 Hz), 3.15 (d, 1H, J=8.0 Hz), 2.86 (d, 1H, J=8.0 Hz), 1.73 (m, 4H). The enantiomer excess (ee) was estimated to be 60% and was directly used in next step without further purification.

### Synthesis of 3-{(4-fluoro-benzyl)-[2-(1-methyl-7-nitro-1-oxo-1λ⁶-benzo[1,2,4]thiadiazin-3-yl)-acetyl]-amino}-7-oxa-bicyclo[2.2.1]heptane-2-carboxylic acid methyl ester (60)

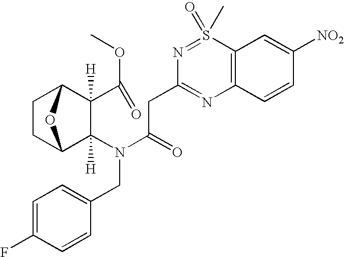

Methyl (1R,2S,3R,4S)-3-[(4-fluorobenzyl)amino]-7-oxabicyclo[2.2.1]heptane-2-carboxylate (59) (121 mg, 0.42 mmol) and (1-methyl-7-nitro-1-oxo-1λ⁶-benzo[1,2,4]thiadiazin-3-yl)-acetic acid ethyl ester (50) (90 mg, 0.29 mmol) were dissolved in anhydrous toluene (3 mL) and then, triethylamine (0.12 mL, 0.87 mmol) was added. The resulting mixture was stirred at 110° C. for 24 h in a sealed tube, and then at 115° C. for 48 h. The mixture was allowed to cool to room temperature, and then concentrated in vacuo to give the desired product, 3-{(4-fluoro-benzyl)-[2-(1-methyl-7-nitro-1-oxo-1λ⁶-benzo[1,2,4]thiadiazin-3-yl)-acetyl]-amino}-7-oxa-bicyclo[2.2.1]heptane-2-carboxylic acid methyl ester (60), which was used directly in the next step without further purification. LC-MS (ES⁺) (m/e): 545.4 [M+H⁺] (exact ms: 544.1).

### Synthesis of 3-(4-fluoro-benzyl)-6-hydroxy-5-(1-methyl-7-nitro-1-oxo-1λ⁶-benzo[1,2,4]thiadiazin-3-yl)-11-oxa-3-aza-tricyclo[6.2.1.0^{2,7}]undec-5-en-4-one (61)

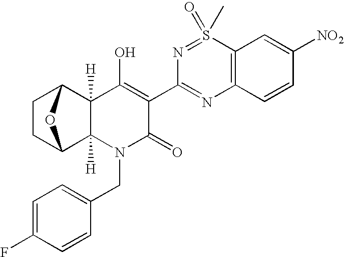

The above crude product 3-{(4-fluoro-benzyl)-[2-(1-methyl-7-nitro-1-oxo-1λ⁶-benzo[1,2,4]thiadiazin-3-yl)-acetyl]-amino}-7-oxa-bicyclo[2.2.1]heptane-2-carboxylic acid methyl ester (60) was dissolved in ethanol (1.5 mL), sodium ethoxide solution (21 wt % in ethanol, 0.4 mL) was added and the mixture was stirred at 25° C. for 30 minutes. The reaction mixture was quenched by 0.5 M aqueous hydrochloric acid solution, and extracted with ethyl acetate. The combined organic layers were washed brine, dried over magnesium sulfate and filtered. The filtrate was concentrated in vacuo to afford the desired product, 3-(4-fluoro-benzyl)-6-hydroxy-5-(1-methyl-7-nitro-1-oxo-1λ⁶-benzo[1,2,4]thiadiazin-3-yl)-11-oxa-3-aza-tricyclo[6.2.1.02,7]undec-5-en-4-one (61), which was used directly in the next step without further purification. LC-MS (ES⁺) (m/e): 513.3 [M+H⁺] (exact ms: 512.1).

### Synthesis of 5-(7-amino-1-methyl-1-oxo-1λ⁶-benzo[1,2,4]thiadiazin-3-yl)-3-(4-fluoro-benzyl)-6-hydroxy-11-oxa-3-aza-tricyclo[6.2.1.0^{2,7}]undec-5-en-4-one (62)

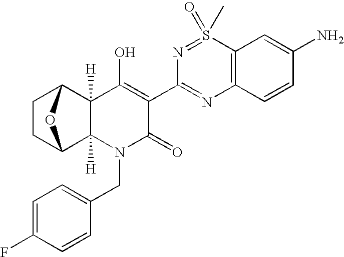

To a stirred solution of 3-(4-fluoro-benzyl)-6-hydroxy-5-(1-methyl-7-nitro-1-oxo-1λ⁶-benzo[1,2,4]thiadiazin-3-yl)-11-oxa-3-aza-tricyclo[6.2.1.0^{2,7}]undec-5-en-4-one (61) in anhydrous THF-MeOH (1:1, 10 mL), Raney-Nickel catalyst (50% slurry in H₂O, 0.4 mL) and anhydrous hydrazine (0.3 mL neat) were added sequentially. The resulting mixture was stirred at room temperature for 1.5 h, passed through a plug of celite and rinsed with 10% MeOH in DCM. The filtrate was concentrated in vacuo to afford the desired product, 5-(7-amino-1-methyl-1-oxo-1λ⁶-benzo[1,2,4]thiadiazin-3-yl)-3-(4-fluoro-benzyl)-6-hydroxy-11-oxa-3-aza-tricyclo[6.2.1.0^{2,7}]undec-5-en-4-one (62), which was used directly in the next step without further purification. LC-MS (ES⁺) (m/e): 483.4 [M+H⁺] (exact ms: 482.1).

### Synthesis of N-{3-[3-(4-fluoro-benzyl)-6-hydroxy-4-oxo-11-oxa-3-aza-tricyclo[6.2.1.0^{2,7}]undec-5-en-5-yl]-1-methyl-1-oxo-1λ⁶-benzo[1,2,4]thiadiazin-7-yl}-methanesulfonamide (63)

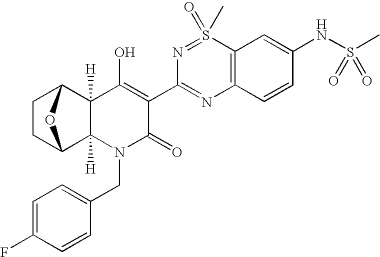

To a stirred solution of above obtained intermediate 5-(7-amino-1-methyl-1-oxo-1λ⁶-benzo[1,2,4]thiadiazin-3-yl)-3-(4-fluoro-benzyl)-6-hydroxy-11-oxa-3-aza-tricyclo[6.2.1.0^{2,7}]undec-5-en-4-one (62) in anhydrous acetone (5 mL), pyridine (0.3 mL, 3.7 mmol) and methanesulfonyl chloride (0.24 mL, 3.1 mmol) were added sequentially. The resulting mixture was stirred at room temperature for 3 h, and then concentrated in vacuo. The residue was purified by prep-HPLC [Column Luna 5μ C18 (2) 100 Å AXIA 150×21.2 mm, 5 micron, 30%-95% CH₃CN in H₂O with 0.04% TFA as gradient, 7 minutes run with 30 mL/min flow rate] to afford the desired product, N-{3-[3-(4-fluoro-benzyl)-6-hydroxy-4-oxo-1′-oxa-3-aza-tricyclo[6.2.1.0^{2,7}]undec-5-en-5-yl]-1-methyl-1-oxo-1λ⁶-benzo[1,2,4]thiadiazin-7-yl}-methanesulfonamide (63) (15 mg, 9% overall yield for 4 steps) as a yellow solid. ¹H NMR (400 MHz, DMSO-d₆) δ: 1.40-1.62 (m, 4H), 2.82 (d, 1H, J=8.8 Hz), 3.09 (s, 3H), 4.26 (d, 1H, J=15.2 Hz), 4.52 (d, 1H, J=5.6 Hz), 4.61 (br, s, 2H), 5.03 (d, 1H, J=15.2 Hz), 7.13 (m, 2H), 7.28 (m, 2H), 7.41 (m, 1H), 7.53 (m, 1H), 7.76 (d, 1H, J=2.0 Hz), 10.08 (s, 1H); LC-MS (ES⁺) (m/e): 561.2 [M+H⁺] (exact ms: 560.1). This compound contains 4 stereo-isomers.

### EXAMPLE 2-3

### Scheme 2g Describes a Similar Procedure, Which was Used to Synthesize Compound 68 of Formula I

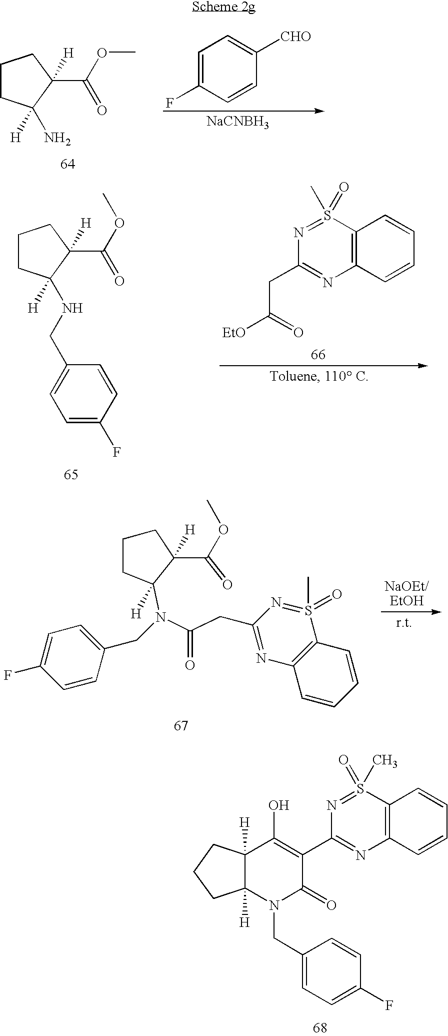

### Synthesis of 2-(4-fluoro-benzylamino)-cyclopentanecarboxylic acid methyl ester (65)

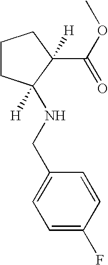

2-Amino-cyclopentanecarboxylic acid methyl ester (64) (0.6 g, 4.2 mmol) was dissolved in methanol (5 mL) and 4-fluoro-benzaldehyde (0.4 mL, 4.2 mmol) was added. The mixture was stirred at room temperature for 5 minutes. HOAc (0.5 mL) was then added followed by sodium cyanoborohydride (0.53 g, 8.4 mmol) and the reaction mixture was stirred at 25° C. for 16 hours. The reaction mixture was poured onto a mixture of aqueous saturated sodium bicarbonate solution (200 mL) and ethyl acetate (300 mL). After shaking, the separated aqueous layer was extracted with EtOAc (4×50 mL), and the combined organic layers were washed with brine one time and dried over anhydrous MgSO₄, filtered and concentrated down in vacuo to give crude product (65) as a light yellow liquid (1 g). LC-MS (ES⁺) (m/e): 252.0 [M+1]⁺ (exact ms: 251.1). ¹H NMR (400 MHz, CDCl₃) δ: 1.53-1.73 (m, 2H), 1.84-1.92 (m, 2H), 1.98-2.08 (m, 2H), 2.95-3.00 (q, 1H, J=7.2 Hz), 3.28-3.34 (q, 1H, J=7.2 Hz), 3.70 (s, 3H), 3.76 (m, 2H), 7.0 (dd, 2H, J₁=9.2 Hz, J₂=18 Hz), 7.26-7.29 (m, 2H).

### Synthesis of 1-(4-fluoro-benzyl)-4-hydroxy-3-(1-methyl-1-oxo-1λ⁶-benzo[1,2,4]thiadiazin-3-yl)-1,4a,5,6,7,7a-hexahydro-[1]pyrindin-2-one (68)

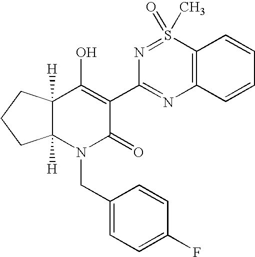

2-(4-Fluoro-benzylamino)-cyclopentanecarboxylic acid methyl ester (65) (140 mg, 0.56 mmol) and 1-methyl-1-oxo-1λ⁶-benzo[1,2,4]thiadiazin-3-yl)-acetic acid sulfoximide ethyl ester 66 (150 mg, 0.56 mmol) were dissolved in 3 mL of toluene and the mixture was heated to 110° C. for 4 days, then concentrated down to remove toluene to give the crude product 67.

The above brown crude 67 was dissolved in EtOH (3 mL). Sodium ethoxide (0.5 mL, 0.7 mmol) was added at room temperature. After 1.5 hours stirring at room temperature, the reaction mixture was acidified with 2N HCl to pH=1˜2 and the aqueous layer was extracted with EtOAc (4×50 mL). The combined organic layers were washed with brine one time and dried over MgSO₄, filtered and concentrated down in vacuo to give crude product as yellow-brown liquid which was further purified by reverse phase HPLC purification to give the pure desired product (68) (21 mg, 10% isolated yield for two steps). ¹H NMR (400 MHz, DMSO-d₆) δ: 1.49-1.66 (m, 2H), 1.67-1.70 (m, 2H), 1.93-2.06 (m, 2H), 7.06 (dd, 2H, J₁=10.0 Hz, J₂=17.2 Hz), 7.30 (m, 2H), 7.37-7.41 (m, 1H), 7.58 (q, 1H, J₁=7.2 Hz, J₂=14.0 Hz), 7.86 (q, 1H, J₁=8.0 Hz, J₂=12.4 Hz), 7.94 (dd, 1H, J₁=4.8 Hz, J₂=12.4 Hz), LC-MS (ES⁺) (m/e): 440.2 [M+1]⁺ (exact ms: 439.1).

### EXAMPLE 2-4

### Scheme 2h describes a similar procedure which was used to synthesize N-{3-[6-(S)-Ethyl-1-(4-fluoro-benzyl)-4-hydroxy-2-oxo-1,2,5,6-tetrahydro-pyridin-3-yl]-1-methyl-1-oxo-1λ⁶-benzo[1,2,4]thiadiazin-7-yl}-methanesulfonamide (75) of Formula I

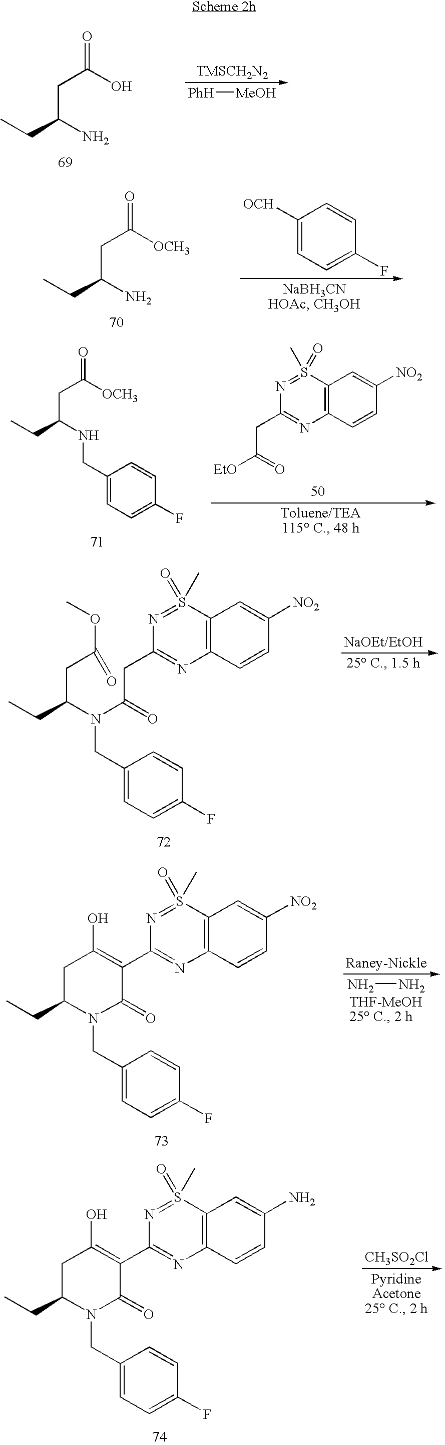

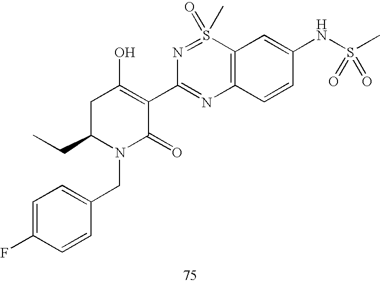

### (S)-3-Amino-pentanoic acid methyl ester (70)

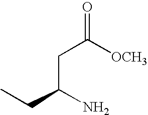

To a stirred solution of 3-amino-pentanoic acid (69) (1.0 g, 8.54 mmol) in anhydrous methanol and benzene (1:1, 14 mL) at 0° C. under a nitrogen atmosphere, TMS-diazomethane (Aldrich) (2.0 M in Hexanes, 10.7 mL, 21.3 mmol) was added dropwise. The resulting mixture was allowed to warm to 25° C., stirred for 1 h, and then concentrated in vacuo to give the desired product, (S)-3-amino-pentanoic acid methyl ester (70) (1.10 g, 99% yield) as a colorless oil. ¹H NMR (400 MHz, CDCl₃) δ: 0.96 (t, 3H, J=7.2 Hz), 1.50 (m, 2H), 2.48 (m, 2H), 3.13 (m, 1H), 3.71 (s, 3H).

### (S)-3-(4-Fluoro-benzylamino)-pentanoic acid methyl ester (71)

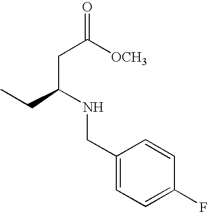

4-Fluoro-benzaldehyde (Aldrich) (0.9 mL, 8.54 mmol) was added to a solution of (S)-3-amino-pentanoic acid methyl ester (70) (1.10 g, 8.54 mmol) in anhydrous CH₃OH (16 mL) at 25° C. under a nitrogen atmosphere. After stirring for 10 minutes, glacial acetic acid (Fisher Scientific) (0.75 mL) and sodium cyanoborohydride (Aldrich) (1.75 g, 21.4 mmol) were added sequentially, and the resulting mixture was stirred at 25° C. for 18 h. The reaction mixture was poured into saturated NaHCO₃ solution and extracted with EtOAc. The combined organic layers were washed with brine, dried over Na₂SO₄ and concentrated in vacuo. The residue was dried in high vacuum to afford desired product, (S)-3-(4-fluoro-benzylamino)-pentanoic acid methyl ester (71) (1.63 g, 80% yield) as a colorless oil. ¹H NMR (400 MHz, CDCl₃) δ: 0.95 (t, 3H, J=7.2 Hz), 1.56 (m, 2H), 2.49 (t, 2H, J=6.8 Hz), 2.98 (m, 1H), 3.69 (s, 3H), 3.77 (m, 2H), 4.68 (s, 1H), 7.00 (m, 2H), 7.30 (m, 2H).

### (S)-3-{(4-Fluoro-benzyl)-[2-(1-methyl-7-nitro-1-oxo-1λ⁶ benzo[1,2,4]thiadiazin-3-yl)-acetyl]-amino}-pentanoic acid methyl ester (72)

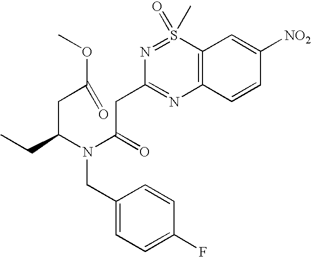

(S)-3-(4-Fluoro-benzylamino)-pentanoic acid methyl ester (71) (378 mg, 1.58 mmol) and (1-methyl-7-nitro-1-oxo-1λ⁶-benzo[1,2,4]thiadiazin-3-yl)-acetic acid ethyl ester (50) (crude compound, 1.58 mmol) were dissolved in anhydrous toluene (4 mL) in a sealed tube, triethylamine (0.6 mL) was added. The resulting mixture was stirred at 115° C. for 48 h, and LC-MS spectrum showed the completion of the reaction. The mixture was allowed to cool to room temperature, and then concentrated in vacuo to give the desired product, (S)-3-{(4-fluoro-benzyl)-[2-(1-methyl-7-nitro-1-oxo-1λ⁶-benzo[1,2,4]thiadiazin-3-yl)-acetyl]-amino}-pentanoic acid methyl ester (72), which was used directly in the next step without purification. LC-MS (ESI⁺) (m/e): 505.2 [M+1] (exact ms: 504.15).

### 6-(S)-Ethyl-1-(4-fluoro-benzyl)-4-hydroxy-3-(1-methyl-7-nitro-1-oxo-1λ⁶-benzo[1,2,4]thiadiazin-3-yl)-5,6-dihydro-1H-pyridin-2-one (73)

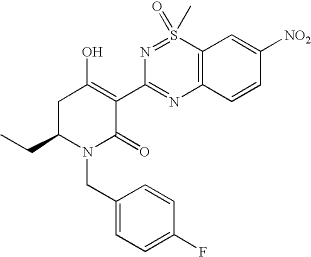

All amounts of above crude product (S)-3-{(4-fluoro-benzyl)-[2-(1-methyl-7-nitro-1-oxo-1λ⁶-benzo[1,2,4]thiadiazin-3-yl)-acetyl]-amino}-pentanoic acid methyl ester (72) was dissolved in ethanol (6 mL), sodium ethoxide solution (21 wt % in ethanol, 1.0 mL) was added and the mixture was stirred at 25° C. for 2.5 h. The reaction mixture was quenched by 0.5 M aqueous hydrochloric acid solution, and extracted with ethyl acetate. The combined organic layers were washed brine, dried over magnesium sulfate and filtered. The filtrate was concentrated in vacuo to afford the desired product, 6-(S)-ethyl-1-(4-fluoro-benzyl)-4-hydroxy-3-(1-methyl-7-nitro-1-oxo-1λ⁶-benzo[1,2,4]thiadiazin-3-yl)-5,6-dihydro-1H-pyridin-2-one (73), which was used directly in the next step without purification. LC-MS (ESI⁺) (m/e): 473.1 [M+1]⁺ (exact ms: 472.12).

### 3-(7-Amino-1-methyl-1-oxo-1λ⁶-benzo[1,2,4]thiadiazin-3-yl)-6-ethyl-1-(4-fluoro-benzyl)-4-hydroxy-5,6-dihydro-1H-pyridin-2-one (74)

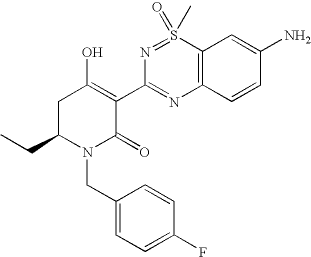

To a stirred solution of above obtained intermediate 6-ethyl-1-(4-fluoro-benzyl)-4-hydroxy-3-(1-methyl-7-nitro-1-oxo-1λ⁶-benzo[1,2,4]thiadiazin-3-yl)-5,6-dihydro-1H-pyridin-2-one (73) in anhydrous THF-MeOH (1:1, 10 mL), Raney-Nickel catalyst (50% slurry in H₂O, 1.1 mL) and anhydrous hydrazine (0.5 mL) were added sequentially. The resulting mixture was stirred at room temperature for 30 minutes, passed through a plug of celite and rinsed with 10% MeOH in DCM. The filtrate was concentrated in vacuo to afford the desired product, 3-(7-amino-1-methyl-1-oxo-1λ⁶-benzo[1,2,4]thiadiazin-3-yl)-6-(S)-ethyl-1-(4-fluoro-benzyl)-4-hydroxy-5,6-dihydro-1H-pyridin-2-one (74), which was used directly in the next step without purification. LC-MS (ESI⁺) (m/e): 443.3 [M+1]⁺ (exact ms: 442.2).

### N-{3-[6-(S)-Ethyl-1-(4-fluoro-benzyl)-4-hydroxy-2-oxo-1,2,5,6-tetrahydro-pyridin-3-yl]-1-methyl-1-oxo-1λ⁶-benzo[1,2,4]thiadiazin-7-yl}-methanesulfonamide (75)

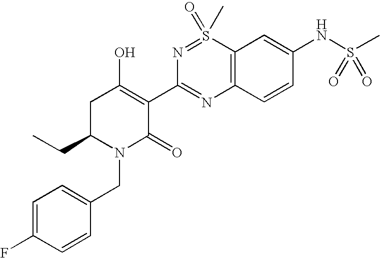

To a stirred solution of above intermediate 3-(7-amino-1-methyl-1-oxo-1λ⁶-benzo[1,2,4]thiadiazin-3-yl)-6-(S)-ethyl-1-(4-fluoro-benzyl)-4-hydroxy-5,6-dihydro-1H-pyridin-2-one (74) (190 mg, 0.43 mmol) in anhydrous acetone (5 mL), pyridine (0.17 mL, 2.15 mmol) and methanesulfonyl chloride (0.1 mL, 1.29 mmol) were added sequentially. The resulting mixture was stirred at room temperature for 1.5 h, and then concentrated in vacuo. The residue was purified by prep-HPLC [Column Luna 5μ C18 (2) 100 Å AXIA 150×21.2 mm, 5 micron, 30-95% in 7 min@30 mL/min flow rate, 0.05% trifluoroacetic acid in acetonitrile/0.05% trifluoroacetic acid in water] to afford the desired product, N-{3-[6-(S)-ethyl-1-(4-fluoro-benzyl)-4-hydroxy-2-oxo-1,2,5,6-tetrahydro-pyridin-3-yl]-1-methyl-1-oxo-1λ⁶-benzo[1,2,4]thiadiazin-7-yl}-methanesulfonamide (75) (10 mg, 4.4% overall yield for last 4 steps) as a yellow solid. ¹H NMR (400 MHz, DMSO-d₆) (a mixture of two diastereomers) δ: 0.82 (m, 3H), 1.48 (m, 1H), 1.59 (m, 1H), 2.27 (m, 1H), 2.66 (m, 1H), 3.26 (m, 1H), 4.04 (m, 1H), 5.11 (m, 1H), 7.10 (m, 2H), 7.33 (m, 2H), 7.39 (d, 1H, J=9.2 Hz), 7.53 (m, 1H), 7.75 (m, 1H), 10.06 (s, 1H); LC-MS (ESI⁺) (m/e): 521.3 [M+1]⁺ (exact ms: 520.13).

Scheme 3 provides a general procedure that can be used to prepare compound 77 of Formula I.

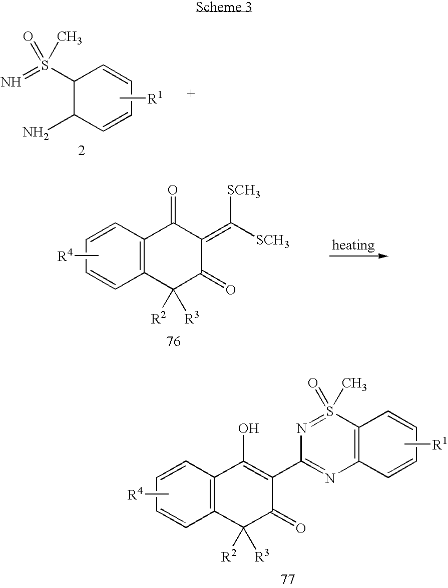

In this general procedure, the sulfoximine 2 can react with compound 76 in the temperature range from 30° C. to 120° C. in a solvent selected from dioxane, THF, toluene, etc. to form the desired product 77 of Formula I.

Schemes 4-22 describe the procedures for synthesis of intermediates.

Scheme 4 describes the synthesis of 2-aminophenyl-5-methyl-sulfoximide (8).

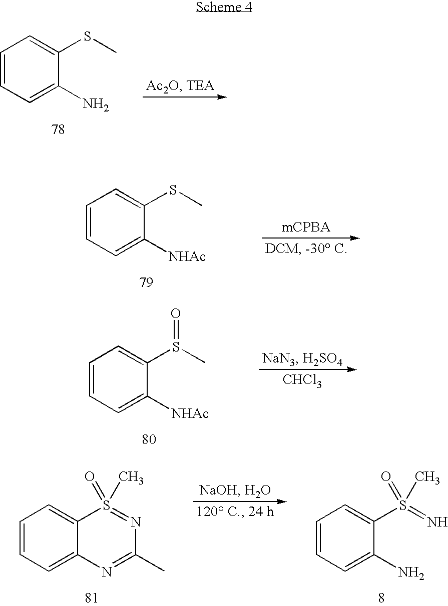

### Synthesis of N-(2-Methylsulfanyl-phenyl)-acetamide (79)

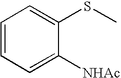

2-Methylsulfanyl-phenylamine (78) (7.5 g, 6.8 mL, 53.9 mmol) was dissolved in dry CHCl₃ (100 mL) and to this solution was added Ac₂O (10.2 mL) and Et₃N (11.2 mL). The clear yellow solution was stirred under N₂ for 1 h, and then refluxed in a 90° C. oil bath for 1 h. The resulting clear, amber solution was cooled to room temperature and poured into a separatory funnel containing saturated aqueous NaHCO₃ (250 mL). The aqueous layer was extracted twice with DCM, and the combined organic extracts were dried over MgSO₄, filtered, and concentrated in vacuo to give a yellow solid. This crude material was recrystallized from boiling heptane to give 9.5 g (97% yield) of N-(2-methylsulfanyl-phenyl)-acetamide (79) as off-white needles. The ¹H-NMR is identical to that reported by Andersen et al. See Andersen et al., J. Org. Chem., 53, 4667-4675 (1988).

### Synthesis of N-(2-methanesulfinyl-phenyl)-acetamide (80)

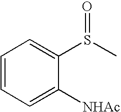

N-(2-methylsulfanyl-phenyl)-acetamide (79) (4.5 g, 24.8 mmol) was dissolved in DCM (100 mL) and cooled to −30° C. in an acetonitrile/dry ice bath. To this solution was added dropwise a solution of 5.7 g (24.8 mmol) mCPBA dissolved in DCM (50 mL) at −30° C. The appearance of the solution quickly changed from clear yellow to very cloudy white. After 15 minutes, saturated aqueous NaHCO₃ (250 mL) was added and the mixture was warmed to room temperature and stirred for 30 minutes. The aqueous layer was then extracted three times with DCM, dried over MgSO₄, filtered, and concentrated in vacuo to give the desired product (80) (4.83 g, 99% yield) as a gold oil that required no further purification. ¹H NMR (400 MHz, CDCl₃) δ: 10.54 (broad s, 1H), 8.49 (d, 1H, J=8.4 Hz), 7.49 (td, 1H, J₁=8.0 Hz, J₂=1.4 Hz), 7.28 (d, 1H, J=1.7 Hz), 7.11-7.15 (m, 1H), 2.94 (s, 3H), 2.24 (s, 3H); LC-MS (ESI⁺): m/e=197.7 [M+1]⁺ (Exact Mass: 197.0). The above synthesis was described in Wnuk et al., J. Org. Chem., 55, 4757-4760 (1990).

### Synthesis of 1,3-dimethyl-1λ⁴-benzo[1,2,4]thiadiazine 1-oxide (81)

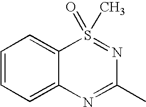

N-(2-methanesulfinyl-phenyl)-acetamide (80) (3.12 g, 15.8 mmol) was dissolved in CHCl₃ (60 mL) and concentrated H₂SO₄ (40 mL). To this solution NaN₃ (2.14 g, 32.9 mmol) were added in small portions in the hood. The reaction is biphasic; the upper, organic layer is a clear pale yellow color and the lower H₂SO₄ layer gradually turns to a clear, deep violet color. The mixture was heated to 40° C. for 2 h and then stirred at room temperature overnight. The CHCl₃ layer was then removed. The H₂SO₄ layer was poured over 200 mL of crushed ice, cooled to 0° C. and neutralized to pH=7 with solid KOH (the mixture slowly changes from violet to red to pink as the pH increases). The aqueous layer is then filtered if necessary and extracted twice with DCM. The combined organic layers are dried over MgSO₄, filtered and concentrated in vacuo to give the desired product (81) (2.46 g, 80% yield) as a yellow powder that required no further purification. ¹H NMR (400 MHz, CDCl₃) δ: 2.42 (s, 3H), 3.48 (s, 3H), 7.39 (t, 1H, J=7.8 Hz), 7.47-7.53 (m, 1H), 7.68 (td, 1H, J₁=7.5 Hz, J₂=1.7 Hz), 7.77 (dd, 1H, J₁=7.7 Hz, J₂=1.4 Hz); LC-MS (ESI⁺): m/e=195.2 [M+1]⁺ (Exact Mass: 194.05). The above synthesis was described in Cohnen, E. & Mahnke, J. Chem. Ber., 105, 757-769 (1972).

### Synthesis of Intermediate 8

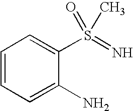

Compound 81 (92.6 mg, 0.48 mmol) was suspended in 10% aqueous NaOH (1 mL) and H₂O (1 mL) and sonicated to give a cloudy slurry. The reaction mixture was heated overnight at 120° C. with stirring. After cooling down, the reaction mixture was diluted with H₂O (2 mL) and the product was extracted with CH₂Cl₂ (6 mL×3). The combined organic layers were dried over Na₂SO₄, filtered, concentrated in vacuo to give 59.8 mg of clear oil as desired product (8) in 73% yield. LC-MS (ES⁺): m/e=171.2 [M+1]⁺ (exact mass: 170.05). ¹H NMR (400 MHz, CDCl₃) δ: 7.79 (dd, 1H, J₁=8 Hz, J₂=1.6 Hz), 7.33 (dt, 1H, J=7.8 Hz, J₂=1.6 Hz), 6.81 (dt, 1H, J₁=7.8 Hz, J₂=1 Hz), 6.74 (dd, 1H, J₁=8.4 Hz, J₂=1.2 Hz), 3.13 (s, 3H). The synthesis for this step is referred to the procedure described in J. Org. Chem., 38(1), 25 (1973).

Scheme 5 describes the synthesis of 2-amino-5-nitrophenyl-S-methyl-sulfoximide (18).

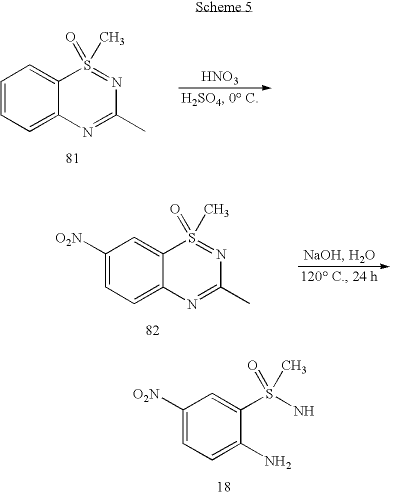

### Synthesis of 1,3-Dimethyl-7-nitro-1λ⁴-benzo[1,2,4]thiadiazine 1-oxide (82)

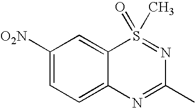

1,3-Dimethyl-1λ⁴-benzo[1,2,4]thiadiazine 1-oxide (81) (1.34 g, 6.9 mmol) was dissolved with help of sonication in concentrated H₂SO₄ (8 mL) and cooled to 0° C. A solution of KNO₃ (0.82 g, 8.1 mmol) in concentrated H₂SO₄ (4 mL) was added dropwise to the previous solution at 0° C. After 3 h sitting in a refrigerator, the reaction mixture was poured onto ice and neutralized with K₂CO₃. The product was extracted with CH₂Cl₂ and EtOAc. The combined organic layer was dried with MgSO₄, filtered and concentrated in vacuo to give the desired product (82) (0.754 g, 46% yield) as a yellow solid that was directly used in next step without further purification. ¹H NMR (400 MHz, CDCl₃) δ: 8.71 (d, 1H, J=2 Hz), 8.46 (dd, 1H, J₁=9.4 Hz, J₂=2.6 Hz), 7.56 (d, 1H, J=8.8 Hz), 3.58 (s, 3H), 2.47 (s, 3H); LC-MS (ESI⁺): m/e=240.1 [M+1]⁺ (Exact Mass: 239.04). The above synthesis was referred to the literature procedure described in Cohnen & Mahnke, J. Chem. Ber., 105, 757-769 (1972).

### Synthesis of Intermediate 18

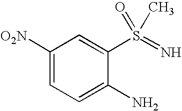

A solution of NaOH (0.71 g) dissolved in a mixture of solvents of EtOH and H₂O (4:1) was added into a flask that contains compound 82 (1.24 g, 5.93 mmol). The reaction solution changed color to dark red. After stirring at room temperature for 4 h, the product was extracted with CH₂Cl₂. The combined organic layer was dried with MgSO₄, filtered and concentrated in vacuo to give the desired product (18) (0.953 g, 74.7% crude yield) as an orange powder that was directly used in next step without further purification. LC-MS (ES⁺): m/e=216.1 [M+1]⁺ (Exact Mass: 215.04). ¹H NMR (400 MHz, CD₃OD) δ: 8.67 (d, 1H, J=2.4 Hz), 8.19 (dd, 1H, J₁=8.8 Hz, J₂=2.8 Hz), 6.96 (d, 1H, J=9.2 Hz), 3.19 (s, 3H).

Scheme 6 describes the synthesis of S-methyl-S-[2-amino-5-(methylsulfonamide)phenyl]-sulfoximide (85).

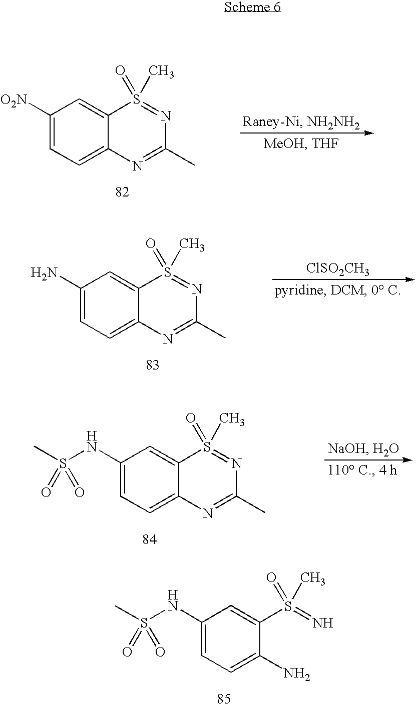

### Synthesis of 1,3-dimethyl-1-oxo-1,6-benzo[1,2,4]thiadiazin-7-ylamine (83)

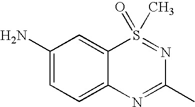

1,3-Dimethyl-7-nitro-1λ⁴-benzo[1,2,4]thiadiazine 1-oxide (82) (0.754 g) was dissolved in THF (30 mL) and MeOH (30 mL). Raney nickel slurry (0.67 mL) was added immediately followed by hydrazine (40 μL), and an immediate effervescence was observed. LC-MS analysis after 4 minutes revealed a major peak with desired product mass of 210.2. TLC result also confirmed the completion of the reaction. Under a nitrogen stream, the reaction was filtered through a thick plug of Celite which was rinsed several times with ethyl acetate. The filtrate was concentrated in vacuo to give a tan paste from which the product was precipitated by the addition of DCM. Filtration gave 0.461 g (70%) of the desired compound (83). ¹H NMR (400 MHz, CD₃OD) δ: 2.25 (s, 3H), 3.54 (s, 3H), 7.06-7.09 (m, 2H), 7.15-7.17 (m, 1H); LC-MS (ESI⁺): m/e=210.2 [M+1]⁺ (Exact Mass: 209.1).

### Synthesis of N-(1,3-dimethyl-1-oxo-1λ⁶-benzo[1,2,4]thiadiazin-7-yl)-methanesulfonamide (84)

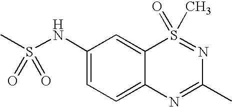

1,3-Dimethyl-1-oxo-1λ⁶-benzo[1,2,4]thiadiazin-7-ylamine (83) (0.461 g, 2.2 mmol) and pyridine (0.36 mL, 4.4 mmol) were dissolved in DCM (10 mL) and cooled to 0° C. Methanesulfonyl chloride (0.2 mL, 2.64 mmol) was then slowly added and the reaction mixture was stirred at 0° C. overnight. The reaction was then partitioned between saturated aqueous NaHCO₃ and DCM, extracted three times with DCM, dried over MgSO₄, filtered and concentrated in vacuo to give 0.315 g (50%) of a dark red foam as the desired product (84) that was directly used in next step without further purification. LC-MS (ESI⁺): m/e=288.0 [M+1]⁺ (Exact Mass: 287.0).

### Synthesis of S-methyl-S-[2-amino-5-(methylsulfonamide)phenyl]-sulfoximide (85)

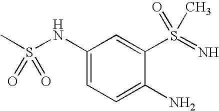

N-(1,3-dimethyl-1-oxo-1λ⁶-benzo[1,2,4]thiadiazin-7-yl)-methanesulfonamide (84) (0.315 g, 1.1 mmol) was suspended in 10% NaOH (1 mL) and refluxed for 4 h. The reaction was then cooled to room temperature, diluted with saturated aqueous NH₄Cl (2 mL), and concentrated in vacuo. The tan-colored residue was dissolved in a small amount of MeOH and purified by flash column chromatography to give the desired product (85). ¹H NMR (400 MHz, CD₃OD) δ: 2.90 (s, 3H), 3.12 (s, 3H), 6.86 (d, 1H, J=8.5 Hz), 7.27 (dd, 1H, J₁=8.5 Hz, J₂=2.3 Hz), 7.62 (d, 1H, J=2.4 Hz); LC-MS (ESI⁺): m/e=263.7 [M+1]⁺ (Exact Mass: 263.0).

Scheme 7 describes the synthesis of (1-Methyl-7-nitro-1-oxo-1λ⁶-benzo[1,2,4]thiadiazin-3-yl)-acetic acid ethyl ester (50).

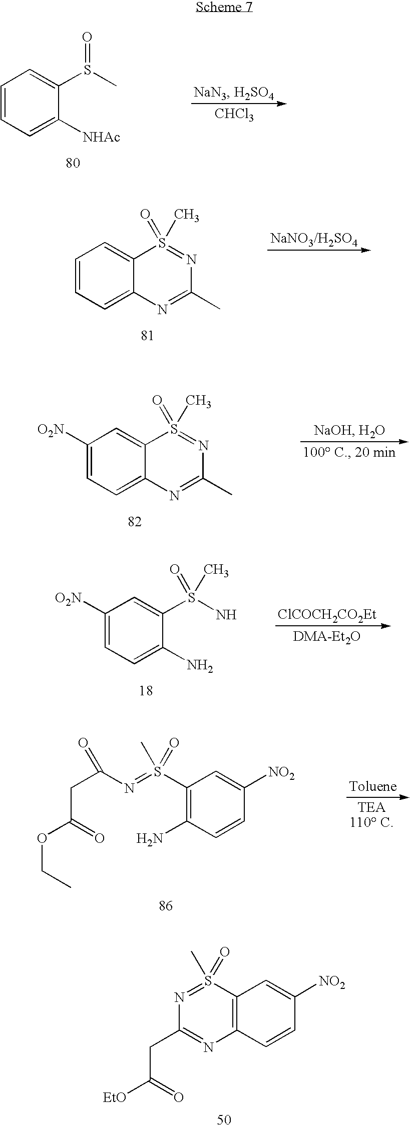

### Synthesis of 1,3-dimethyl-1λ⁴-benzo[1,2,4]thiadiazine 1-oxide (81)

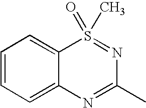

N-(2-Methanesulfinyl-phenyl)-acetamide (80) (23 g, 120 mmol) was dissolved in CHCl₃ (160 mL) and concentrated H₂SO₄ (100 mL). To this solution, NaN₃ (15.2 g, 240 mmol) was added in small portions in the hood. The mixture was stirred at 40° C. for 3 h and then was allowed to cool to room temperature and stirred overnight. The reaction mixture was poured into a mixture of crushed ice and water, cooled to 0° C. and neutralized to pH=7 with solid NaOH. The aqueous layer was extracted twice with DCM. The combined organic layers were dried over MgSO₄, filtered and concentrated in vacuo to give the crude product. The yellow solid was triturated with DCM/Hexanes to give the pure product, 1,3-dimethyl-1λ⁴-benzo[1,2,4]thiadiazine 1-oxide (81) (14.0 g, 61%) as an off-white solid. ¹H NMR (400 MHz, CDCl₃) δ: 2.42 (s, 3H), 3.49 (s, 3H), 7.40 (m, 1H), 7.46 (dd, 1H, J=8.0, 0.8 Hz), 7.68 (m, 1H), 7.78 (dd, 1H, J=8.4, 1.6 Hz).

### Synthesis of 1,3-dimethyl-7-nitro-1λ⁴-benzo[1,2,4]thiadiazine 1-oxide (82)

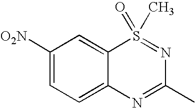

1,3-Dimethyl-1λ⁴-benzo[1,2,4]thiadiazine 1-oxide (81) (1.50 g, 7.72 mmol) was dissolved in concentrated H₂SO₄ (8 mL) and cooled to 0° C. KNO₃ (937 mg, 9.28 mmol) was dissolved in 4 mL of concentrated H₂SO₄ and the resulting solution was added dropwise to the previous solution and stirred at 0° C. for 6 h. The reaction mixture was poured into ice-H₂O, carefully neutralized with NH₄OH, and then extracted with DCM. The combined organic layers were dried over MgSO₄, filtered and concentrated in vacuo to give the desired product, 1,3-dimethyl-7-nitro-1λ⁴-benzo[1,2,4]thiadiazine 1-oxide (82) (1.78 g, 97% yield) as a yellow solid. ¹H NMR (400 MHz, CDCl₃) δ: 2.47 (s, 3H), 3.59 (s, 3H), 7.57 (d, 1H, J=8.8 Hz), 8.46 (m, 1H), 8.72 (d, 1H, J=2.0 Hz); LC-MS (ESI⁺): m/e=240.1 [M+1]⁺ (Exact Mass: 239.0).

### Synthesis of 2-amino-5-nitro-benzenesulfoximide (18)

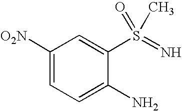

The suspension of 1,3-dimethyl-7-nitro-1λ⁴-benzo[1,2,4]thiadiazine 1-oxide (82) (478 mg, 2.0 mmol) in 5 mL of 4% NaOH aqueous solution was heated to 100° C. and stirred for 20 min. The suspension became brown solution upon heating and then yellow solid precipitated out. H₂O was added to the reaction mixture, and the solid was collected by filtration, rinsed with H₂O and then dried in high vacuum to give the desired product, 2-amino-5-nitro-benzenesulfoximide (18) (360 mg, 84%) as a yellow solid. ¹H NMR (400 MHz, DMSO-d₆) δ: 3.06 (d, 3H, J=0.8 Hz), 4.68 (s, 1H), 6.90 (d, 1H, J=9.2 Hz), 7.51 (broad s, 2H), 8.10 (dd, 1H, J=9.2, 2.4 Hz), 8.45 (d, 1H, J=2.8 Hz); LC-MS (ESI⁺): m/e=216.1 [M+1]⁺ (Exact Mass: 215.04).

### Synthesis of N-malonyl-2-amino-5-nitro-benzenesulfoximide (86)

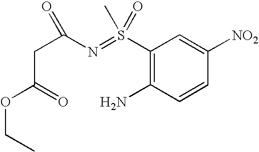

To a solution of 2-amino-5-nitro-benzenesulfoximide (18) (200 mg, 0.93 mmol) in anhydrous DMA-Et₂O (1:1 mixture, 1.5 mL), ethylmalonyl chloride (0.12 mL, 0.98 mmol) was added. The mixture was stirred at room temperature for 16 h, diluted with ethyl acetate, washed with 1.0 M aqueous HCl solution and brine, dried over MgSO₄ and filtered. The filtrate was concentrated in vacuo, and the crude residue was triturated with 40% EtOAc/Hexanes to give the desired product, N-malonyl-2-amino-5-nitro-benzenesulfoximide (86) (155 mg, 51%) as a yellow solid. ¹H NMR (400 MHz, CDCl₃) δ: 1.31 (m, 3H), 3.42-3.52 (m, 5H), 4.21 (q, 2H, J=6.4 Hz), 6.14 (broad s, 2H), 6.82 (d, 1H, J=9.6 Hz), 8.24 (dd, 1H, J=9.6, 2.4 Hz), 8.77 (d, 1H, J=2.4 Hz); LC-MS (ESI⁺): m/e=330.1 [M+1]⁺ (Exact Mass: 329.3).

### Synthesis of (1-methyl-7-nitro-1-oxo-1λ⁶-benzo[1,2,4]thiadiazin-3-yl)-acetic acid ethyl ester (50)

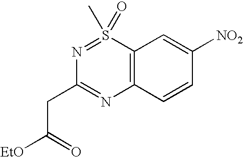

N-malonyl-2-amino-5-nitro-benzenesulfoximide (86) (155 mg, 51%) (195 mg, 0.59 mmol) was dissolved in anhydrous toluene (8 mL) in a sealed tube, and triethylamine (0.33 mL, 2.37 mmol) was added to this solution. The resulting mixture was stirred at 110° C. for 20 h. The mixture was allowed to cool to room temperature, diluted with EtOAc, washed with 1.0 M aqueous HCl solution and brine, dried over MgSO₄ and filtered. The filtrate was concentrated in vacuo to give the desired product, (1-methyl-7-nitro-1-oxo-1λ⁶-benzo[1,2,4]thiadiazin-3-yl)-acetic acid ethyl ester (50), which was used directly in the next step without purification. LC-MS (ESI⁺): m/e=312.1 [M+1]⁺ (Exact Mass: 311.3).

Scheme 8 describes the synthesis of (1-methyl-1-oxo-1λ⁶-benzo[1,2,4]thiadiazin-3-yl)-acetic acid sulfoximide ethyl ester (66).

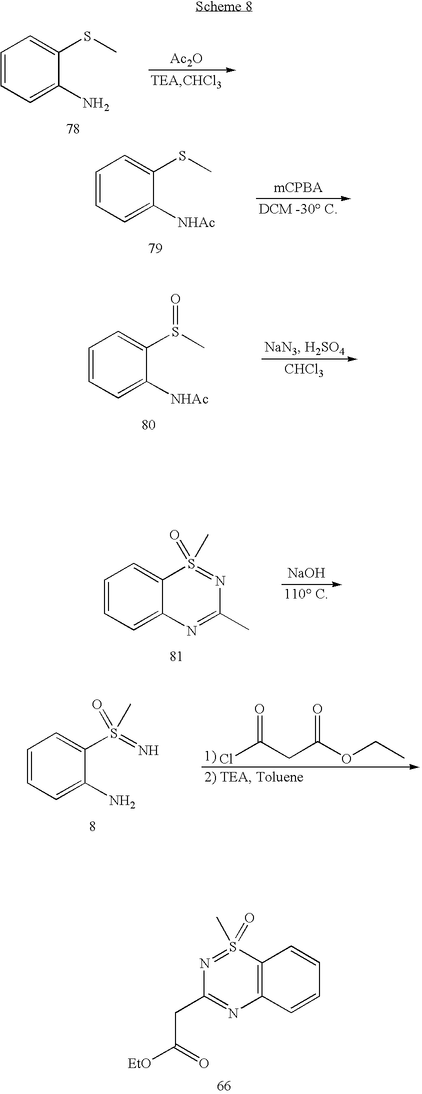

### Synthesis of N-(2-methylsulfanyl-phenyl)-acetamide (79)

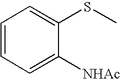

2-Methylsulfanyl-phenylamine (78) (15 g, 0.11 mol) was dissolved in 200 mL of dry CHCl₃ and into this solution was added 20.7 mL of Ac₂O (0.22 mol) and 23 mL of (0.17 mol) Et₃N. The resulted clear solution was stirred at room temperature for 90 minutes, then heated to reflux for 90 minutes. After cooled to room temperature, the reaction mixture was poured onto 250 mL of saturated aqueous NaHCO₃ solution. The aqueous layer was separated and extracted with DCM (3×100 mL), and the combined organic layer was dried over MgSO₄, filtered and concentrated in vacuo to give the desired product (79) as colorless oil (19.8 g). ¹H NMR (400 MHz, CDCl₃) δ: 2.24 (s, 3H), 2.40 (s, 3H), 7.06 (dd, 1H, J₁=7.2 Hz, J₂=5.6 Hz), 7.29 (d, 1H, J=7.0 Hz), 7.46 (d, 1H, J=7.0 Hz), 8.29 (d, 1H, J=7.6 Hz), LC-MS (ES⁺) (m/e): 182.2 [M+1]⁺ (exact ms: 181.1).

### Synthesis of N-(2-methanesulfinyl-phenyl)-acetamide (80)

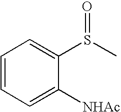

N-(2-Methylsulfanyl-phenyl)-acetamide (79) (crude from last step, 0.11 mol) was dissolved in 300 mL of DCM and cooled to −30° C. To this solution was added dropwise a solution of 24.6 g (0.11 mol) of 3-chloroperoxybenzoic acid (mcpba) in 150 mL of DCM in 45 minutes. After stirring for 45 minutes, 300 mL of saturated aqueous NaHCO₃ solution was added and the mixture was warmed to room temperature, stirred for another 45 minutes. The organic phase was separated and the aqueous layer was extracted with DCM a few times until no product in aqueous layer. The combined organic layers were washed with brine one time and dried over anhydrous MgSO₄, filtered and concentrated down in vacuo to give the desired product (80) as white solid (21 g, 97% yield for two steps). ¹H NMR (400 MHz, CDCl₃) δ: 2.22 (s, 3H), 2.92 (s, 3H), 7.10 (dd, 1H, J₁=8.0 Hz, J₂=16.4 Hz), 7.25 (d, 1H, J=7.2 Hz), 7.46 (dd, 1H, J₁=8.8 Hz, J₂=17.6 Hz), 8.48 (d, 1H, J=8.8 Hz), 10.53 (s, 1H); LC-MS (ES⁺) (m/e): 198.1 [M+1]⁺ (exact ms: 197.1).

The synthesis of compound 81 was described above in Schemes 4 and 7.

### Synthesis of Compound 8

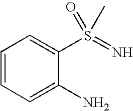

Into compound 81 (2 g, 10.3 mmol) was added 10% aqueous NaOH solution (7 mL, 12.36 mmol), and the mixture was heated to reflux for 3 hours, cooled to room temperature. Saturated NH₄Cl (15 mL) was added, the aqueous layer was extracted with EtOAc (4×150 mL), and the combined organic layers were washed with brine one time and dried over MgSO₄, filtered and concentrated down in vacuo to give the crude product (8) as yellow liquid (1.75 g) in 98% yield. ¹H NMR (400 MHz, CDCl₃) δ: 3.27 (s, 3H), 6.76 (d, 1H, J=8.0 Hz), 6.83 (dd, 1H, J₁=7.6 Hz, J₂=15.6 Hz), 7.36 (dd, 1H, J₁=8.0 Hz, J₂=15.6 Hz), 7.78 (d, 1H, J=7.0 Hz); LC-MS (ES⁺) (m/e): 171.0 [M+1]⁺ (exact ms: 170.1).

### Synthesis of (1-methyl-1-oxo-1l6-benzo[1,2,4]thiadiazin-3-yl)-acetic acid sulfoximide ethyl ester (66)

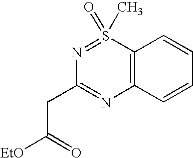

Into compound 8 (0.5 g, 2.64 mmol) was added DMA (4 mL) and Et₂O (4 mL), cooled to 0° C., chlorocarbonyl acetic acid ethyl ester (0.3 mL, 2.64 mmol), the reaction mixture was warmed to room temperature. After stirring for 1.5 hours, 20 mL of H₂O was added, the aqueous layer was extracted with EtOAc (4×150 mL) and the combined organic layers were washed with brine one time and dried over MgSO₄, filtered and concentrated down in vacuo to give the desired uncyclized amide as yellow liquid.

Into the above crude was added toluene (4 mL) followed by TEA (1.2 mL, 8.44 mmol) in a sealed tube. This mixture was heated to 110° C. for 4 days, concentrated down and dried in vacuo to give the desired product (87) as dark brown liquid (400 mg, 57% yield) which was used in next step directly without further purification. LC-MS (ES⁺) (m/e): 267.0 [M+1]⁺ (exact ms: 266.1).

Scheme 9 describes an alternative synthesis of (1R,2R,3S,4S)-3-(4-fluorobenzylamino)-bicyclo[2.2.1]heptane-2-carboxylic acid ethyl ester (91).

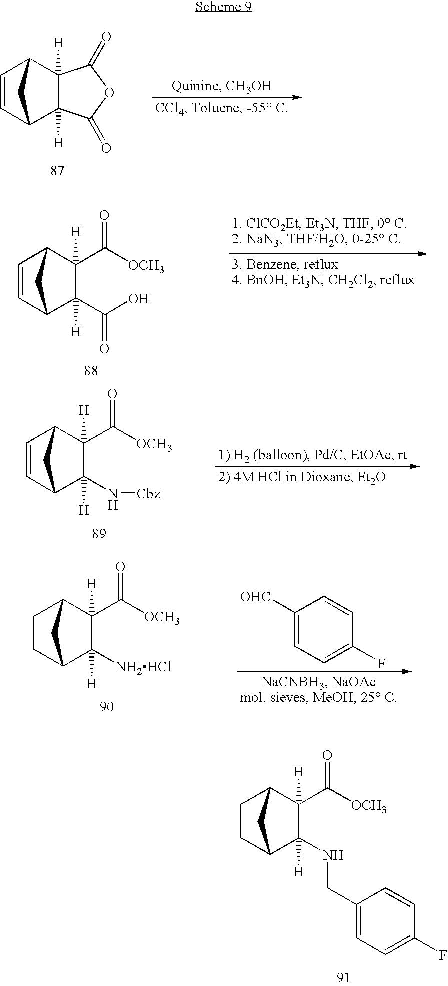

### Step 1: Synthesis of(1R,2S,3R,4S)-3-(methoxycarbonyl)bicyclo[2.2.1]hept-5-ene-2-carboxylic acid (88)

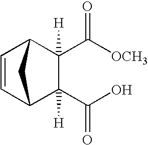

(1R,2R,6S,7S)-4-Oxatricyclo[5.2.1.0^{2,6}]dec-8-ene-3,5-dione (exo) (87) (5 g, 30.45 mmol) was suspended in a 1:1 mixture of toluene and carbon tetrachloride (610 mL). The mixture was stirred for 10 minutes. Quinine (10.87 g, 33.5 mmol) was added and the flask was degassed and backfilled with nitrogen. The solution was chilled to −55° C. While stirring, methyl alcohol (3.7 mL, 91.35 mmol) was added. The mixture continued to stir at −55° C. for 16 h. Upon warming to 25° C., the mixture was concentrated in vacuo to a foam. The foam was dissolved in a mixture of ethyl acetate (400 mL) and 1M aqueous hydrochloric acid (400 mL). The layers were separated and the organic layer was further washed with 1M hydrochloric acid (2×200 mL) and brine (100 mL), dried over anhydrous magnesium sulfate and concentrated in vacuo to afford the desired product, (1R,2S,3R,4S)-3-(methoxycarbonyl)bicyclo[2.2.1]hept-5-ene-2-carboxylic acid (88) (5.95 g, 30.3 mmol, 99% yield) as a clear oil. ¹H NMR (400 MHz, DMSO-d₆) δ: 1.31 (1H, d, J=8.5 Hz), 1.98 (1H, d, J=8.6 Hz), 2.51 (2H, d, J=1.6 Hz), 2.95 (2H, br, s), 3.52 (3H, s), 6.17-6.21 (2H, m), 12.16 (1H, s). This synthesis is referred to in the procedure described in J. Org. Chem., 2000, 65, 6984-6991.

### Step 2: Synthesis of (1S,2R,3S,4R)-3-benzyloxycarbonylamino-bicyclo[2.2.1]hept-5-ene-2-carboxylic acid methyl ester (89)

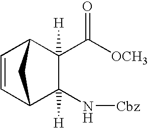

(1R,2S,3R,4S)-3-(Methoxycarbonyl)bicyclo[2.2.1]hept-5-ene-2-carboxylic acid (88) (5.9 g, 30 mmol) was dissolved in anhydrous tetrahydrofuran (133 mL). The flask was degassed and backfilled with nitrogen and the mixture was chilled to 0° C. Triethyl amine (12.64 mL, 90 mmol) was added followed by the dropwise addition of ethyl chloroformate (5.72 mL, 60 mmol) with vigorous stirring. Immediate precipitation was observed. The mixture continued to stir at 0° C. for 1 h. Sodium azide (5.86 g, 90 mmol) was dissolved in water (40 mL) and added to the reaction mixture at 0° C. The mixture was stirred at 0° C. for 5 minutes. The ice bath was removed. The mixture was warmed to 25° C. and continued to stir for 2 h. The mixture was poured into water (300 mL) and the product was extracted into ethyl acetate (300 mL). The organic layer was further washed with ½ saturated sodium bicarbonate solution (2×100 mL), brine (100 mL), dried over magnesium sulfate and concentrated in vacuo to afford a light brown oil. The oil was dissolved in anhydrous benzene (66 mL) and refluxed while stirring under nitrogen for 2 h. Upon cooling to 25° C., the solution was concentrated in vacuo to afford a light brown oil. The oil was dissolved in methylene chloride (40 mL) and benzyl alcohol (3.41 mL, 33 mmol) was added followed by triethyl amine (8.44 mL, 60 mmol). The mixture was refluxed under nitrogen for 16 h. Upon cooling to 25° C., the solution was concentrated in vacuo to afford a thick oil. Purification twice by flash column chromatography (one with 3:1 hexane/ethyl acetate and the other one with 2:4:1 methylene chloride/pentane/diethyl ether) afforded the desired product, methyl (1S,2R,3S,4R)-3-{[(benzyloxy)carbonyl]amino}bicyclo[2.2.1]hept-5-ene-2-carboxylate (89) (6.95 g, 23.09 mmol, 77% yield) as a pale yellow oil. ¹H NMR (400 MHz, CDCl₃) δ: 1.59 (1H, d, J=9.3 Hz), 1.96 (1H, d, J=9.3 Hz), 2.66 (1H, d, J=7.9 Hz), 2.75 (1H, s), 2.96 (1H, s), 3.59 (3H, s), 4.01 (1H, t, J=8.5 Hz), 5.09 (2H, q, J=10.4 Hz), 5.46 (1H, d, J=9.4 Hz), 6.17-6.22 (2H, m), 7.29-7.36 (5H, m). LC-MS (ES⁺) (m/e): 302.2 [M+1]⁺ (70%), 603.5 [2M+1]⁺ (20%) (exact mass: 301.1). The synthesis is referred to in the procedure described in Synthesis, 2001, 11, 1719-1730.

### Step 3: Synthesis of(1R,2R,3S,4S)-3-amino-bicyclo[2.2.1]heptane-2-carboxylic acid methyl ester hydrochloride salt (90)

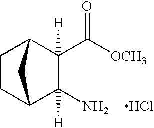

Methyl (1S,2R,3S,4R)-3-{[(benzyloxy)carbonyl]amino}bicyclo[2.2.1]hept-5-ene-2-carboxylate (89) (1 g, 3.32 mmol) was dissolved in ethyl acetate (15 mL). 5% Palladium on carbon (120 mg) was added. The flask was degassed and backfilled with hydrogen gas via balloon. The mixture was stirred at 25° C. for 16 h. The mixture was passed through a plug of celite and the filtrate was concentrated in vacuo to afford a thick clear oil. The oil was dissolved in diethyl ether (10 mL) and added dropwise with vigorous stirring to a mixture of 4 M HCl in dioxane (1.8 mL) in diethyl ether (18 mL). The desired product began to precipitate as a white solid. Additional diethyl ether (10 mL) was added and the mixture continued to stir for 10 minutes. The precipitate was collected by vacuum filtration, rinsed with additional diethyl ether (2×8 mL). The solid was further dried in vacuo for 1 h. to afford the desired product, methyl (1R,2R,3S,4S)-3-aminobicyclo[2.2.1]heptane-2-carboxylate hydrochloride (90) (0.64 g, 3.11 mmol, 94% yield) as a white powder. ¹H NMR (400 MHz, DMSO-d₆) δ: 1.17-1.27 (3H, m), 1.40-1.61 (2H, m), 1.91 (1H, d, J=10.7 Hz), 2.36 (1H, d, J=4.1 Hz), 2.44 (1H, d, J=3.1 Hz), 2.75 (1H, d, J=7.8 Hz), 3.30-3.38 (1H, m), 3.61 (3H, s), 8.05 (3H, bs). LC-MS (ES⁺) (m/e): 170.3 [M+1]⁺ (100%), 339.3 [2M+1]⁺ (50%) (exact mass of the free amine: 169.1).

The above reaction was scaled up as follows.

Methyl (1S,2R,3S,4R)-3-{[(benzyloxy)carbonyl]amino}bicyclo[2.2.1]hept-5-ene-2-carboxylate (89) (17.72 g, 58.87 mmol) was dissolved in ethyl acetate (265 mL). 5% Palladium on carbon (2.56 g) was added. The flask was degassed and backfilled with hydrogen gas via balloon. The mixture stirred at 25° C. for 6.5 h. The mixture was passed through a plug of celite and the filtrate was concentrated in vacuo to afford a thick clear oil. The oil was dissolved in ethyl acetate (200 mL) and added with vigorous stirring to a mixture of 4 M HCl in dioxane (30 mL) in diethyl ether (350 mL). The desired product began to precipitate/crystallize as a white solid. The mixture sat still for 20 minutes. The precipitate was collected by vacuum filtration and re-dissolved in boiling ethyl acetate (1500 mL). Upon cooling, the product began to crystallize. The mixture sat still for 48 h. The crystals were collected by filtration, rinsed with diethyl ether, and further dried in vacuo for 1 h to afford the desired product, methyl (1R,2R,3S,4S)-3-aminobicyclo[2.2.1]heptane-2-carboxylate hydrochloride (90) (6.9 g, 33.55 mmol, 57% yield) as a white crystalline, powder.

### Step 4: Synthesis of(1R,2R,3S,4S)-3-(4-fluoro-benzylamino)-bicyclo[2.2.1]heptane-2-carboxylic acid methyl ester (91)

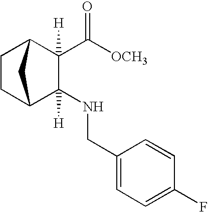

Methyl (1R,2R,3S,4S)-3-aminobicyclo[2.2.1]heptane-2-carboxylate hydrochloride (90) (0.5 g, 2.43 mmol) was dissolved in methanol (12 mL). Sodium acetate (0.4 g, 4.86 mmol) was added followed by 4 Å powdered molecular sieves (0.5 g) and 4-fluoro-benzaldehyde (0.302 g, 2.43 mmol). Sodium cyanoborohydride (0.305 g, 4.86 mmol) was added and the mixture was stirred at 25° C. for 16 h. The mixture was poured into a mixture of aqueous saturated sodium bicarbonate solution (200 mL) and ethyl acetate (300 mL). After shaking, both layers were passed through a plug of celite. The separated organic phase was further washed with aqueous saturated sodium bicarbonate solution (100 mL), brine solution (100 mL), dried over magnesium sulfate and concentrated in vacuo to afford the crude product, methyl (1R,2R,3S,4S)-3-[(4-fluorobenzyl)amino]bicyclo[2.2.1]heptane-2-carboxylate (91) (0.663 g, 2.39 mmol, 98% yield) as a clear oil. LC-MS (ES⁺) (m/e): 278.2 [M+1]⁺ (100%) (exact mass: 277.2)

The above reaction was scaled up according to the following procedures.

Methyl (1R,2R,3S,4S)-3-aminobicyclo[2.2.1]heptane-2-carboxylate hydrochloride (90) (6.9 g, 33.55 mmol) was dissolved in methanol (165 mL). Sodium acetate (5.5 g, 67.1 mmol) was added followed by 4 Å powdered molecular sieves (6.9 g) and 4-fluoro-benzaldehyde (4.164 g, 33.55 mmol). Sodium cyanoborohydride (4.22 g, 67.1 mmol) was added and the mixture was stirred at 25° C. for 16 h. The mixture was poured into ethyl acetate (400 mL) and shaken with ½ saturated aqueous sodium bicarbonate solution (300 mL). Both layers were passed through a plug of celite. The separated organic phase was further washed with ½ saturated aqueous sodium bicarbonate solution (2×200 mL), brine (100 mL), dried over magnesium sulfate and concentrated in vacuo to afford the crude product, methyl (1R,2R,3S,4S)-3-[(4-fluorobenzyl)amino]bicyclo[2.2.1]heptane-2-carboxylate (91) (9.3 g, 33.53 mmol, >99% yield) as a clear oil.

Scheme 10 describes another method for the synthesis of (1R,2R,3S,4S)-3-(4-fluorobenzylamino)-bicyclo[2.2.1]heptane-2-carboxylic acid ethyl ester (49).

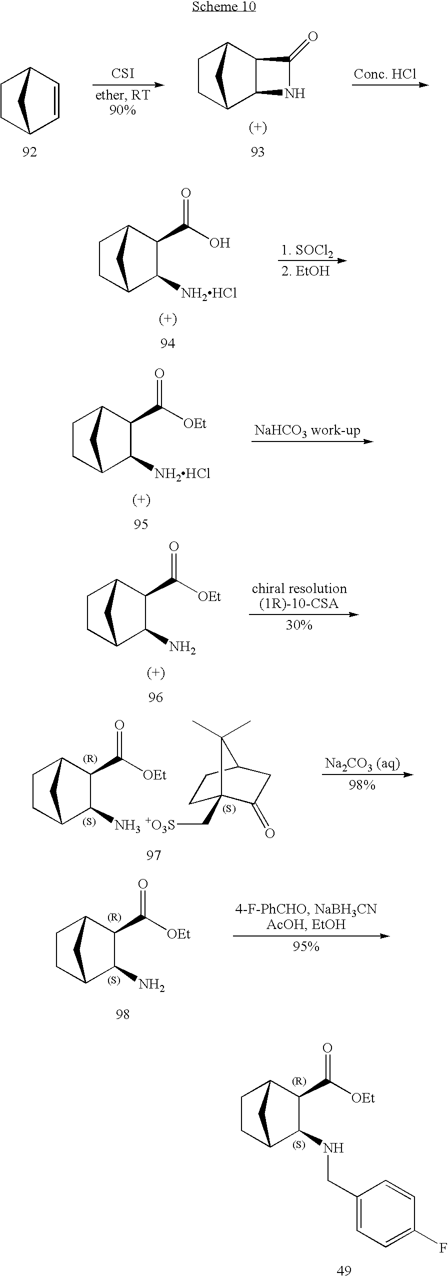

### Step 1: Synthesis of 3-aza-tricyclo[4.2.1.0^{2,5}]nonan-4-one (93) (racemic di-exo)

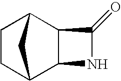

To a solution of bicyclo[2.2.1]hept-2-ene (92) (30.92 g, 325.1 mmol) in diethyl ether (150 mL) at 0° C. was added a solution of chlorosulfonylisocyanate (CSI) (29.0 mL, 327.3 mmol) in diethyl ether (50 mL) dropwise over 10 minutes. The mixture was slowly allowed to warm to 25° C. for 12 h (overnight). The reaction mixture was cooled to 0° C. and a solution of sodium sulfite (56.89 g) in water (440 mL) was added dropwise with stirring. The mixture was allowed to warm to 25° C. To this mixture was added 10% aqueous potassium hydroxide solution (350 mL) until pH=7-8. The organic layer was separated and the aqueous layer was extracted with diethyl ether (200 mL×2). The combined organic layers were washed with brine solution (200 mL×1), dried over anhydrous magnesium sulfate, concentrated in vacuo, and dried under vacuum to afford the crude 3-aza-tricyclo[4.2.1.0^{2,5}]nonan-4-one as a white solid (93) (35.98 g, 262.3 mmol, 81% yield). ¹H NMR (400 MHz, CDCl₃) δ 1.02-1.11 (2H, m), 1.24 (1H, dt, J₁=10.9 Hz, J₂=1.6 Hz), 1.51-1.72 (3H, m), 2.37-2.37 (1H, m), 2.43-2.44 (1H, m), 2.99-3.00 (1H, m), 3.40 (1H, d, J=3.4 Hz), 5.73 (1H, br, s).

### Step 2: Synthesis of 3-amino-bicyclo[2.2.1]heptane-2-carboxylic acid hydrochloride (94) (racemic di-exo)

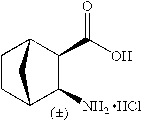

To compound 93 (35.98 g, 262.3 mmol) was added aqueous concentrated hydrochloric acid solution (230 mL). The mixture was stirred at 25° C. for 6 h. The solvent was evaporated in vacuo and the crude compound was dried under high vacuum for 0.5 h. The crude was triturated with acetone and filtered to afford 3-amino-bicyclo[2.2.1]heptane-2-carboxylic acid hydrochloride (94) as a white solid (48.05 g, 250.7 mmol, 96% yield). ¹H NMR (400 MHz, DMSO-d₆) δ 1.15-1.26 (3H, m), 1.42-1.59 (2H, m), 1.87 (1H, d, J=10.3 Hz), 2.33 (1H, d, J=3.4 Hz), 2.45 (1H, d, J=2.3 Hz), 2.67 (1H, d, J=7.6 Hz), 3.23-3.26 (1H, m), 7.93 (3H, br, s), 12.73 (1H, br, s).

### Step 3: Synthesis of 3-amino-bicyclo[2.2.1]heptane-2-carboxylic acid ethyl ester hydrochloride (95) (racemic di-exo)

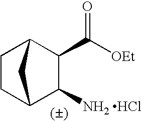

Into a mixture of compound 94 (13.81 g, 72.1 mmol) and absolute ethanol (100 mL) at −10° C. was added thionyl chloride (6.0 mL, 79.8 mmol) dropwise. The reaction mixture was stirred at 0° C. for 1 h, at 25° C. for 3 h, and heated at reflux for 0.5 h. The reaction mixture was concentrated in vacuo and dried under high vacuum to afford the crude 3-amino-bicyclo[2.2.1]heptane-2-carboxylic acid ethyl ester hydrochloride (95) as an off-white solid. ¹H NMR (400 MHz, DMSO-d₆) δ 1.17-1.27 (3H, m), 1.21 (3H, t, J=7.0 Hz), 1.43-1.57 (2H, m), 1.91 (1H, d, J=10.0 Hz), 2.36 (1H, d, J=3.9 Hz), 2.42 (1H, d, J=3.0 Hz), 2.72 (1H, d, J=7.6 Hz), 3.28 (1H, d, J=8.3 Hz), 4.00-4.13 (2H, m), 8.06 (3H, br, s).

### Step 4: Synthesis of 3-amino-bicyclo[2.2.1]heptane-2-carboxylic acid ethyl ester (96) (racemic di-exo)

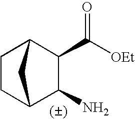

To the compound 95 was added saturated aqueous sodium bicarbonate solution (80 mL) and the mixture was stirred at 25° C. for 0.5 h. The crude product was extracted out with ethyl acetate (100 mL×3). The organic layer was dried over anhydrous magnesium sulfate, concentrated in vacuo and dried under high vacuum for 1 h to afford the crude desired product, 3-amino-bicyclo[2.2.1]heptane-2-carboxylic acid ethyl ester (96), as a brown oil (11.81 g, 64.4 mmol, 89% yield over 2 steps). ¹H NMR (400 MHz, CDCl₃) δ 1.10-1.26 (3H, m), 1.29 (3H, t, J=7.0 Hz), 1.45-1.62 (2H, m), 1.86 (2H, br, s), 1.95 (1H, dt, J₁=10.3 Hz, J₂=1.9 Hz), 2.09 (1H, d, J=4.5 Hz), 2.49 (1H, d, J=4.2 Hz), 2.56 (1H, d, J=9.0 Hz), 3.24 (1H, d, J=7.7 Hz), 4.09-4.21 (2H, m).

### Step 5: Synthesis of(1S,2S,3R,4R)-3-ethoxycarbonyl-bicyclo[2.2.1]hept-2-yl-ammonium (1′S)-(+)-10-camphorsulfonate (97) via chiral resolution

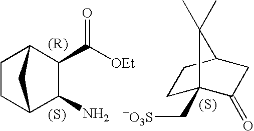

To a solution of compound 96 (racemic di-exo) (4.00 g, 21.8 mmol) in ethyl acetate (120 mL) was added (1S)-(+)-10-camphorsulfonic acid (2.57 g, 11.0 mmol). The mixture was stirred at 25° C. vigorously for 1 h. The solid was filtered (3.23 g, 36%) and recrystallized in ethyl acetate (360 mL) to afford (1S,2S,3R,4R)-3-ethoxycarbonyl-bicyclo[2.2.1]hept-2-yl-ammonium (1′S)-(+)-10-camphorsulfonate (97) as a white solid (2.76 g, 6.64 mmol, 30% yield). ¹H NMR (400 MHz, CDCl₃) δ 0.84 (3H, s), 1.08 (3H, s), 1.30 (3H, t, J=6.9 Hz), 1.32-1.43 (4H, m), 1.58-1.75 (3H, m), 1.89 (1H, d, J=17.7 Hz), 1.95-2.07 (3H, m), 2.33 (1H, dt, J₁=18.4 Hz, J₂=3.9 Hz), 2.53 (1H, s), 2.58-2.65 (1H, m), 2.69 (1H, d, J=2.9 Hz), 2.76-2.79 (2H, m), 3.26 (1H, d, J=14.1 Hz), 3.60 (1H, d, J=7.4 Hz), 4.14-4.27 (2H, m), 7.80 (3H, br, s).

### Step 6: Synthesis of (1R,2R,3S,4S)-3-amino-bicyclo[2.2.1]heptane-2-carboxylic acid ethyl ester (98)

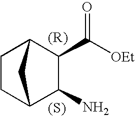

To the above sulfonate salt (97), was added ethyl acetate (28 mL) and saturated aqueous sodium carbonate solution (28 mL) and the mixture was stirred at 25° C. for 0.5 hour. The organic layer was separated and the aqueous layer was extracted with ethyl acetate (50 mL×2). The combined organic layers were dried over anhydrous magnesium sulfate, concentrated in vacuo and dried under high vacuum for 1 h to afford (1R,2R,3S,4S)-3-amino-bicyclo[2.2.1]heptane-2-carboxylic acid ethyl ester (98) as a colorless oil (1.15 g, 6.28 mmol, 29% yield over 2 steps).

### Step 7: Synthesis of (1R,2R,3S,4S)-3-(4-fluoro-benzylamino)-bicyclo[2.2.1]heptane-2-carboxylic acid ethyl ester (49)

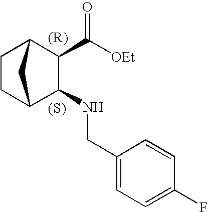

To a solution of compound 98 (1.15 g, 6.28 mmol) in ethanol (30 mL) was added 4-fluorobenzaldehyde (0.68 mL, 6.31 mmol), glacial acetic acid (0.4 mL, 6.99 mmol), and sodium cyanoborohydride (1.04 g, 15.7 mmol) at 25° C. After stirring for 3 hours at room temperature, the mixture was diluted with ethyl acetate (50 mL) and quenched with saturated aqueous sodium bicarbonate (50 mL) for 0.5 h. The mixture was filtered through celite. The organic layer was separated and the aqueous layer was extracted with ethyl acetate (50 mL×2). The combined organic layer was concentrated in vacuo to form a solid which was washed with water, and dried under high vacuum to afford (1R,2R,3S,4S)-3-(4-fluorobenzylamino)-bicyclo[2.2.1]heptane-2-carboxylic acid ethyl ester (49) (1.74 g, 5.97 mmol, 95% yield). ¹H NMR (400 MHz, CDCl₃) δ 1.05-1.16 (2H, m), 1.21 (1H, dt, J₁=8.0 Hz, J₂=1.6 Hz), 1.27 (3H, t, J=7.4 Hz), 1.45-1.61 (2H, m), 1.94 (1H, dt, J₁=10.1 Hz, J₂=1.9 Hz), 2.28 (1H, d, J=3.9 Hz), 2.43 (1H, d, J=3.3 Hz), 2.60 (1H, dd, J₁=8.8 Hz, J₂=1.5 Hz), 2.94 (1H, d, J=7.8 Hz), 3.66 (1H, d, J=13.2 Hz), 3.80 (1H, d, J=13.5 Hz), 4.13 (2H, q, J=7.0 Hz), 6.97 (2H, t, J=8.5 Hz), 7.26 (2H, t, J=7.1 Hz).

Scheme 11 provides a general procedure that can be used to prepare β-aminoester (101).

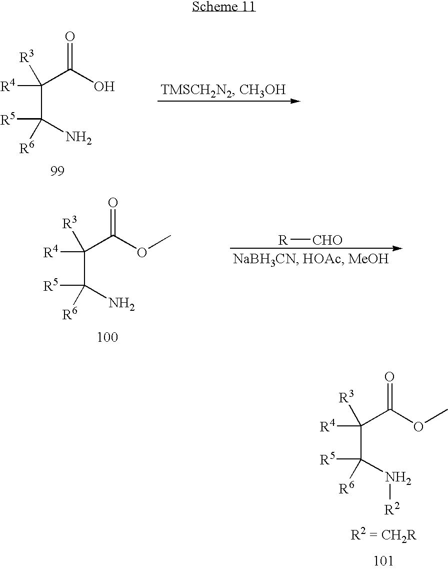

wherein R is an alkyl, aryl or heterocyclic group.

β-amino-acid 99 can be treated with TMSCH₂N₂ to form the corresponding β-amino-ester 100. The amino group of compound 100 can be treated with aldehyde via reductive amination reaction to form compound 101.

### EXAMPLE 10-1

### Scheme 12 Describes the Synthesis of β-aminoester (104)

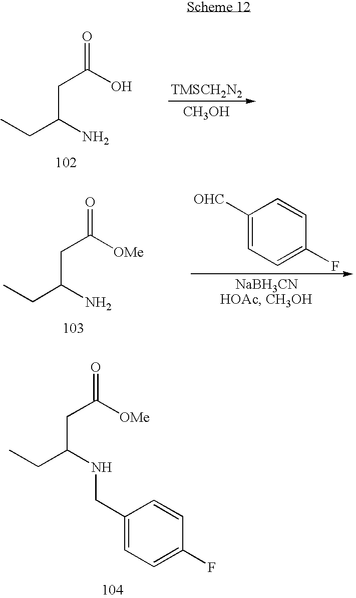

### Synthesis of 3-amino-pentanoic acid methyl ester (103)

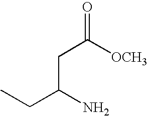

To a stirred solution of 3-amino-pentanoic acid (102) (300 mg, 2.56 mmol) in anhydrous methanol and benzene (1:1, 6 mL) at 0° C. under a nitrogen atmosphere, TMS-diazomethane (Aldrich) (2.0 M in hexane, 3.84 mL, 7.68 mmol) was added dropwise. The resulting mixture was allowed to warm to 25° C., stirred for 1 h, and then concentrated in vacuo to give desired product, 3-amino-pentanoic acid methyl ester (103) (330 mg, 2.56 mmol, 99%) as a colorless oil. ¹H NMR (400 MHz, CDCl₃) δ: 0.96 (t, 3H, J=7.2 Hz), 1.50 (m, 2H), 2.48 (m, 2H), 3.13 (m, 1H), 3.71 (s, 3H).

### Synthesis of 3-(4-fluoro-benzylamino)-pentanoic acid methyl ester (104)

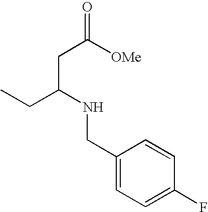

4-Fluoro-benzaldehyde (Aldrich) (0.27 mL, 2.56 mmol) was added to a solution of 3-amino-pentanoic acid methyl ester (103) (330 mg, 2.56 mmol) in anhydrous CH₃OH (10 mL) at 25° C. under a nitrogen atmosphere. After stirring for 10 minutes, glacial acetic acid (Fisher Scientific) (0.5 mL) and sodium cyanoborohydride (Aldrich) (525 mg, 6.4 mmol) were added sequentially, and the resulting mixture was stirred at 25° C. for 18 h. The reaction mixture was poured into saturated sodium bicarbonate solution and extracted with ethyl acetate. The combined organic layers were washed with brine, dried over sodium sulfate and filtered. The filtrate was concentrated in vacuo to afford the desired product, 3-(4-fluoro-benzylamino)-pentanoic acid methyl ester (104) (560 mg, 2.34 mmol, 92% yield) as a colorless oil. ¹H NMR (400 MHz, CDCl₃) δ: 0.95 (t, 3H, J=7.6 Hz), 1.52-1.60 (m, 2H), 2.49 (m, 2H), 3.00 (m, 1H), 3.69 (s, 3H), 3.77 (s, 2H), 7.00 (m, 2H), 7.29 (m, 2H).

When chiral β-aminoacid is used as starting material, the corresponding chiral β-aminoester can be obtained using the above reaction condition. Other β-aminoesters can be prepared in similar way as that described in Scheme 12.

Scheme 13 describes a general method to synthesize the intermediate 107 where alkyl is ethyl.

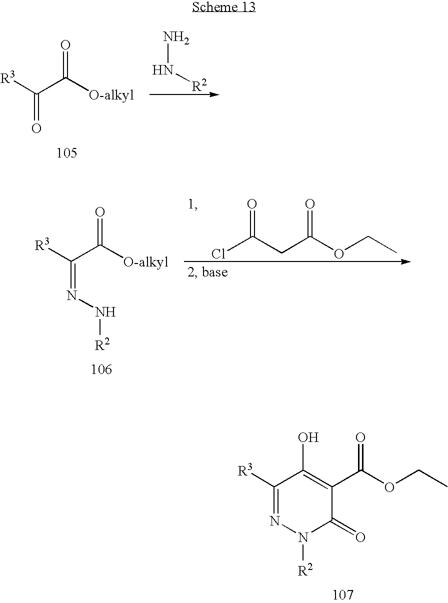

In this general method, α-ketoester (105) can react with hydrazine (NH₂NHR²) under heating condition to form hydrazone (106). Hydrazone 106 can be acylated with ethyl 3-chloro-3-oxo-propionate to form a hydrazide intermediate, which undergoes cyclization under basic condition to form the desired product (107).

### EXAMPLE 5-1

### Scheme 14 Describes the Synthesis of 2-(3,3-dimethyl-butyl)-5-hydroxy-3-oxo-6-thiophen-2-yl-2,3-dihydro-pyridazine-4-carboxylic acid ethyl ester (16)

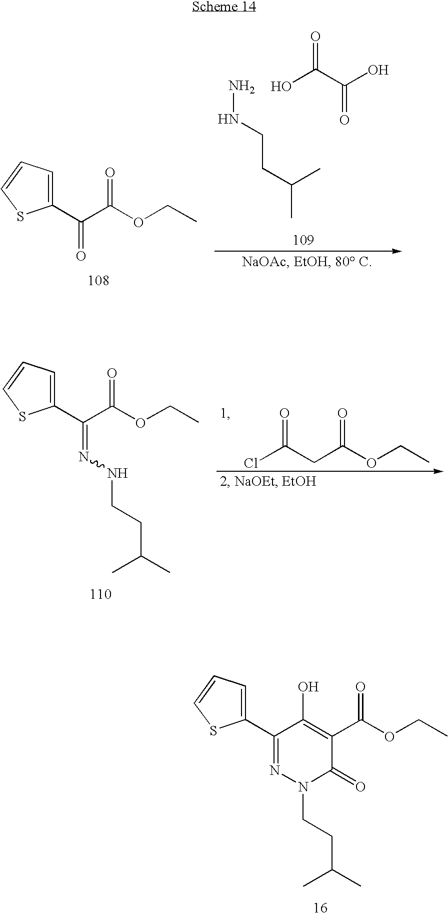

### Synthesis of [(3-methyl-butyl)-hydrazono]-thiophen-2-yl-acetic acid ethyl ester (110)

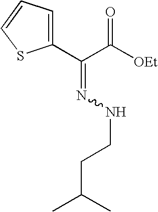

To a solution of oxo-thiophen-2-yl-acetic acid ethyl ester (108) (3.81 g, 20.7 mmol) in absolute ethanol (100 mL), (3-Methyl-butyl)-hydrazine oxalate (109) (3.97 g, 20.7 mmol) was added. The mixture was stirred at 80° C. under N₂ atmosphere for 24 hours. The reaction mixture was concentrated under reduced pressure and the residue was purified by flash chromatography on silica gel to give the desired product (110) (2.08 g, 37%) that was directly used in the next step. LC-MS (ESI⁺): m/e 269.2 [M+1]⁺, 537.4 [2M+1]⁺, 559.0 [2M+Na]⁺ (exact ms: 268.12).

### Synthesis of 5-hydroxy-2-(3-methyl-butyl)-3-oxo-6-thiophen-2-yl-2,3-dihydro-pyridazine-4-carboxylic acid ethyl ester (16)

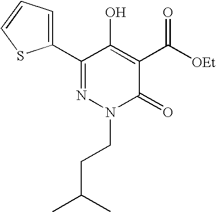

To a solution of [(3-Methyl-butyl)-hydrazono]-thiophen-2-yl-acetic acid ethyl ester (110) (2.08 g, 7.76 mmol) in anhydrous dioxane under N₂ atmosphere, ethyl malonyl chloride (90%, Alfa Aesar) (1.32 mL, 9.31 mmol) was added. The reaction mixture was stirred at 100° C. for 20 minutes, cooled to rt, diluted with EtOAc and washed with aqueous NaHCO₃ and brine, dried over Na₂SO₄.

The solvent was removed under reduced pressure, and the residue was dissolved in EtOH (25 mL) at room temperature, sodium ethoxide solution (Aldrich) (21 wt % in ethanol, 3.50 mL, 9.3 mmol) was added, and the resulting mixture was stirred for 30 minutes. Aqueous HCl (5%, 6.3 mL) was added to the reaction mixture in a period of 10 minutes, followed by liquid-liquid extraction with H₂O/EtOAc. The combined organic layer was washed with brine, dried over Na₂SO₄. Solvent was removed under reduced pressure and the residue was purified by flash chromatography on silica gel to give the desired product (16) (1.67 g, 64%) as yellow solid. ¹H NMR (400 MHz, CDCl₃): δ 7.89 (dd, 1H, J=3.6, 1.2 Hz), 7.39 (dd, 1H, J=5.2, 1.2 Hz), 7.10 (dd, 1H, J=5.2, 3.6 Hz), 4.53 (q, 2H, J=7.2 Hz), 4.22 (m, 2H), 1.73 (m, 2H), 1.68 (m, 1H), 1.50 (t, 3H, J=7.2 Hz), 0.99 (d, 6H, J=6.4 Hz); LC-MS (ESI⁺): m/e=337.30 [M+1]⁺ (exact ms: 336.11).

Scheme 15 describes another general method to synthesize the intermediate 107 where alkyl is ethyl.

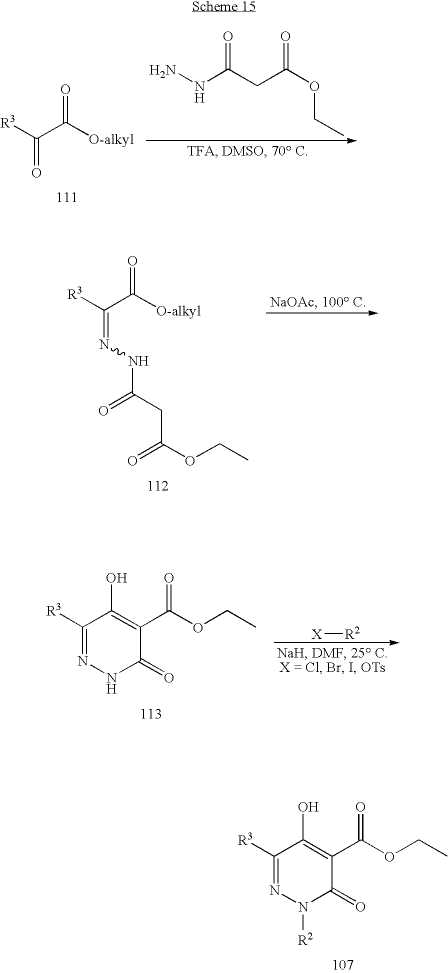

In this general method, α-ketoester (111) can be treated with hydrazinocarbonyl-acetic acid ethyl ester in the presence of TFA with heating to give compound 112. Compound 112 can then undergo cyclization in the presence of NaOAc and heating to form compound 113, which can be alkylated to provide the desired compound 107.

### EXAMPLE 6-1

### Scheme 16 Describes the Synthesis of 2-(3,3-dimethyl-butyl)-5-hydroxy-3-oxo-6-thiophen-2-yl-2,3-dihydro-pyridazine-4-carboxylic acid ethyl ester (19)

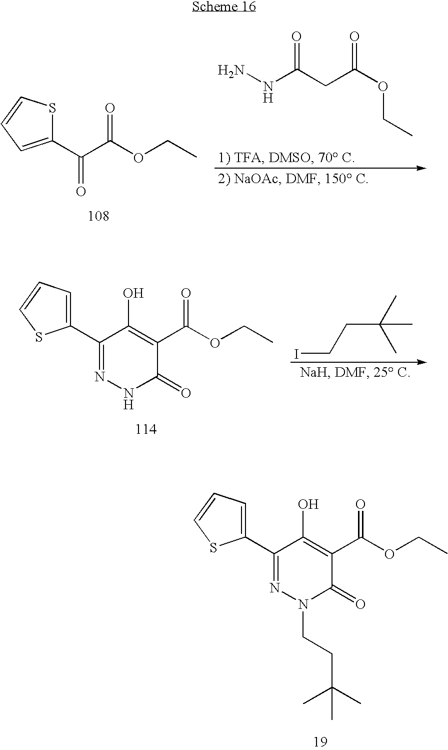

### Synthesis of [(2-Ethoxycarbonyl-acetyl)-hydrazono]-thiophen-2-yl-acetic acid ethyl ester

Oxo-thiophen-2-yl-acetic acid ethyl ester (108) (2 g, 10.86 mmol) was dissolved in anhydrous DMSO (54.3 mL). Hydrazinocarbonyl-acetic acid ethyl ester (1.75 g, 11.95 mmol) was added followed by TFA (0.2 mL). The flask was evacuated and filled with N₂. The mixture was heated at 70° C. for 16 h. Upon cooling to 25° C., the mixture was diluted with EtOAc and washed with 0.1 M HCl (3 times). The organic phase was further washed with brine, dried over MgSO₄ and concentrated in vacuo. Purification of the residue by flash column chromatography (10-15% EtOAc in Hexanes) afforded ([(2-ethoxycarbonyl-acetyl)-hydrazono]-thiophen-2-yl-acetic acid ethyl ester (2.89 g, 9.25 mmol, 85% yield) as a faintly yellow oil that crystallized to a beige, waxy, solid upon standing as [(2-ethoxycarbonyl-acetyl)-hydrazono]-thiophen-2-yl-acetic acid ethyl ester. ¹H NMR (400 MHz, CDCl₃, 10:1 mixture of isomers observed, data for major isomer reported): δ=1.28 (t, 3H, J=6.9 Hz), 1.47 (t, 3H, J=7.1 Hz), 3.80 (s, 2H), 4.22 (q, 2H, J=7.1 Hz), 4.47 (q, 2H, J=7.1 Hz), 7.03 (t, 1H, J=4.2 Hz), 7.33 (d, 1H, J=4.4 Hz), 7.60 (d, 1H, J=3.7 Hz), 11.80 (br s, 1H). ¹³C NMR (100 MHz, CDCl₃): δ=14.2, 14.3, 40.7, 61.5, 62.9, 127.5, 128.1, 129.0, 130.9, 138.2, 160.5, 166.9, 168.5. LC-MS (ESI⁺): m/e=313.1 [M+H⁺] (100%).

### Synthesis of 5-hydroxy-3-oxo-6-thiophen-2-yl-2,3-dihydro-pyridazine-4-carboxylic acid ethyl ester (114)

[(2-Ethoxycarbonyl-acetyl)-hydrazono]-thiophen-2-yl-acetic acid ethyl ester (1 g, 3.2 mmol) was dissolved in DMF (16 mL) and NaOAc (0.525 g, 2.55 mmol) was added. The flask was evacuated and filled with N₂. The mixture was heated at 150° C. for 30 minutes. Upon cooling to 25° C., 1 M HCl (32 mL) was added and the product precipitated. After stirring for 5 minutes, the solid was collected by filtration, washed with 1 M HCl and dried in vacuo for 16 h to afford 5-hydroxy-3-oxo-6-thiophen-2-yl-2,3-dihydro-pyridazine-4-carboxylic acid ethyl ester (114) as a light beige powder (0.68 g, 2.55 mmol, 80% yield). ¹H NMR (400 MHz, DMSO-d₆): δ=1.29 (t, 3H, J=7.3 Hz), 4.30 (q, 2H, J=7.3 Hz), 7.12 (dd, 1H, J=5.4, 3.8 Hz), 7.62 (d, 1H, J=3.8 Hz), 7.80 (d, 1H, J=4.6 Hz), 13.00 (br s, 1H). ¹³C NMR (100 MHz, DMSO-d₆): δ=14.0, 61.6, 107.6, 127.5, 127.8, 127.8, 135.7, 137.1, 158.3, 158.5, 166.2. LC-MS (ESI⁺): m/z=267.1 [M+H]⁺ (100%), 533.2 [2M+H]⁺ (25%).

### Synthesis of 2-(3,3-Dimethyl-butyl)-5-hydroxy-3-oxo-6-thiophen-2-yl-2,3-dihydro-pyridazine-4-carboxylic acid ethyl ester (19)

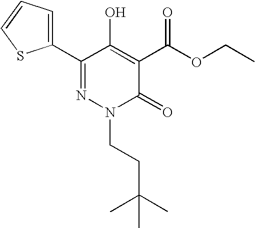

5-hydroxy-3-oxo-6-thiophen-2-yl-2,3-dihydro-pyridazine-4-carboxylic acid ethyl ester (114) (0.5 g, 1.87 mmol) was suspended in dimethylformamide (9.3 mL) followed by addition of sodium hydride (163 mg, 4.11 mmol) at 25° C. The mixture was allowed to stir for 20 minutes after which time 1-iodo-3,3-dimethyl-butane (0.41 g, 2.06 mmol) was added and the mixture was stirred at 25° C. for 4 h. The reaction mixture was then diluted with ethyl acetate (100 mL) and washed twice with 1N HCl (2×100 mL) via extraction. The organic layer was then concentrated to dryness under reduced pressure and the residue was purified by flash column chromatography on silica gel to afford 2-(3,3-dimethyl-butyl)-5-hydroxy-3-oxo-6-thiophen-2-yl-2,3-dihydro-pyridazine-4-carboxylic acid ethyl ester (19) (0.58 g, 89% yield) as a solid. ¹H NMR (400 MHz, CDCl₃) δ: 1.02 (s, 9H), 1.50 (t, 3H, J=7.0 Hz), 1.72-1.76 (m, 2H), 4.20-4.24 (m, 2H), 4.54 (quartet, 2H, J=7.3 Hz), 7.09-7.11 (m, 1H), 7.38-7.39 (m, 1H), 7.88-7.89 (m, 1H), 13.87 (s, 1H), LC-MS (ESI⁺): m/e=351.1 [M+1]⁺ (exact mass: 350.13).

Scheme 17 describes the synthesis of 2-(3,3-dimethyl-butyl)-5-hydroxy-3-oxo-6-pyrrolidin-1-yl-2,3-dihydro-pyridazine-4-carboxylic acid ethyl ester (25).

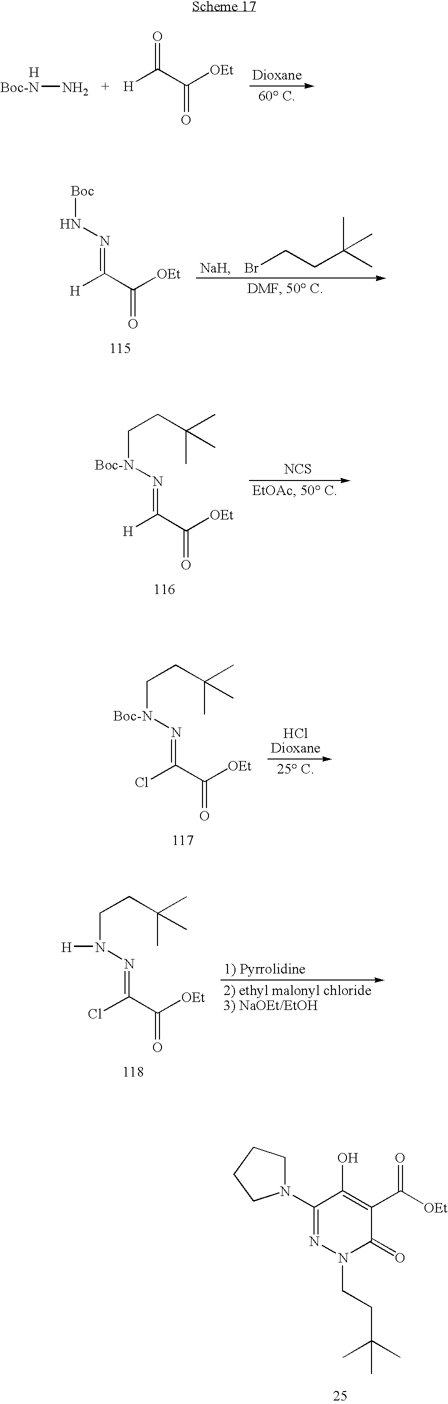

### Synthesis of (tert-butoxycarbonyl-hydrazono)-acetic acid ethyl ester (115)

Ethyl glyoxylate (13.9 mL, 61.3 mmol) was added to a solution of hydrazine carboxylic acid tert-butyl ester (7.35 g, 55.6 mmol) in anhydrous 1,4-dioxane (75 mL) at 25° C. under a nitrogen atmosphere. The resulting mixture was stirred at 60° C. for 15 min. The mixture was cooled to 25° C. and concentrated. The residue was purified by flash chromatography on silica gel to afford the desired product (115) (9.38 g, 13.8% yield) as a white solid. ¹H NMR (400 MHz, CDCl₃) δ: 8.34 (s, 1H), 7.55 (s, 1H), 4.33 (q, 2H, J=7.2 Hz), 1.54 (s, 9H), 1.36 (t, H, J=7.2 Hz).

### Synthesis of [tert-butoxycarbonyl-(3,3-dimethyl-butyl)-hydrazono]-acetic acid ethyl ester (116)

To a solution of compound 115 (9.5 g, 43.9 mmol) in anhydrous DMF (60 mL) at 0° C. under a nitrogen atmosphere, NaH (1.93 g, 48.3 mmol) was added in portions. After stirring at 0° C. for 10 minutes, the reaction mixture was allowed to warm to 25° C. and stirred for 30 minutes. 1-Bromo-3,3-dimethylbutane (8.7 g, 52.7 mmol) was added to the mixture, and stirring was continued at 50° C. for 24 h. The reaction mixture was cooled to 25° C., poured into 0.5 M aqueous HCl (200 mL), and extracted with EtOAc. The combined organic layers were washed with brine, dried over Na₂SO₄ and concentrated. The residue was purified by flash chromatography on silica gel to afford the desired product (116) (8.4 g, 64% yield). ¹H NMR (400 MHz, CDCl₃) δ: 7.11 (s, 1H), 4.33 (q, 2H, J=7.2 Hz), 3.82 (m, 2H), 1.58 (s, 9H), 1.44 (m, 2H), 1.39 (t, 3H, J=7.2 Hz), 0.99 (s, 9H).

### Synthesis of [tert-Butoxycarbonyl-(3,3-dimethyl-butyl)-hydrazono]-chloro-acetic acid ethyl ester (117)

To a solution of compound 116 (8.5 g, 28.3 mmol) in EtOAc (80 mL) under a nitrogen atmosphere, NCS (15.4 g, 113.3 mmol) was added in portions. The resulting mixture was stirred at 50° C. for 22 h. The reaction mixture was then cooled to 25° C. and concentrated. The residue was taken up in hexanes and filtered through filter paper, which was then rinsed with hexanes. The filtrate was concentrated and the residue was purified by flash chromatography on silica gel to afford the desired product (117) (7.2 g, 76% yield). ¹H NMR (400 MHz, CDCl₃) δ: 4.38 (q, 2H, J=7.2 Hz), 4.00 (m, 2H), 1.57 (m, 2H), 1.54 (s, 9H), 1.40 (t, 3H, J=7.2 Hz), 0.97 (s, 9H).

### (3,3-dimethyl-butyl)-hydrazono-chloro-acetic acid ethyl ester (118)

To a solution of compound 117 (5.9 g, 17.7 mmol) in anhydrous 1,4-dioxane (10 mL) under a nitrogen atmosphere, HCl 1,4-dioxane solution (4.0 M, 25 mL, 100 mmol) was added slowly. After stirring at 25° C. for 18 h, the reaction mixture was poured into aqueous NaHCO₃ (150 mL), and extracted with EtOAc. The combined organic layers were washed with brine, dried over Na₂SO₄ and concentrated. The residue was purified by flash chromatography on silica gel to afford the desired product (118) (2.46 g, 59% yield). ¹H NMR (400 MHz, CDCl₃) δ: 6.43 (m, 1H), 4.35 (q, 2H, J=7.2 Hz), 3.56 (m, 2H), 1.57 (m, 2H), 1.38 (t, 3H, J=7.2 Hz), 0.97 (s, 9H).

### 2-(3,3-Dimethyl-butyl)-5-hydroxy-3-oxo-6-pyrrolidin-1-yl-2,3-dihydro-pyridazine-4-carboxylic acid ethyl ester (25)

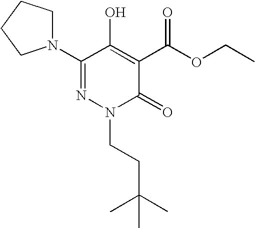

To a solution of compound 118 (1.16 g, 4.95 mmol) in anhydrous 1,4-dioxane (10 mL) under a nitrogen atmosphere, pyrrolidine (1.25 mL, 15 mmol) was added slowly. After stirring at 50° C. for 3 h, the reaction mixture was cooled to 25° C., poured into aqueous NaHCO₃ (150 mL), and extracted with EtOAc. The combined organic layers were washed with brine, dried over Na₂SO₄ and concentrated.

The residue was dissolved in 1,4-dioxane (15 mL), ethyl malonyl chloride (0.68 mL, 5.4 mmol) was added slowly. The resulting mixture was stirred at 85° C. for 20 minutes, then cooled to 25° C., poured into aqueous NaHCO₃ (100 mL), and extracted with EtOAc. The combined organic layers were washed with brine, dried over Na₂SO₄ and concentrated.

The above residue was dissolved in EtOH (20 mL), NaOEt (21 wt %, 2.17 mL, 5.88 mmol) was added slowly. The resulting mixture was stirred at 50° C. for 30 minutes, and then cooled to 25° C., aqueous HCl (2.0 M, 3 mL) was added. The mixture was diluted with EtOAc, washed with H₂O and brine, dried over Na₂SO₄ and concentrated. The residue was purified by flash chromatography on silica gel to afford the desired product (25) (824 mg, 50% yield for 3 steps). ¹H NMR (400 MHz, CDCl₃) δ: 4.48 (m, 2H), 4.03 (m, 1H), 3.92 (m, 1H), 3.72 (m, 2H), 3.52 (m, 2H), 1.94 (m, 2H), 1.87 (m, 2H), 1.67 (m, 2H), 1.48 (m, 3H), 0.98 (m, 9H); LC-MS (ESI⁺): m/e 338.4 [M+1]⁺ (exact ms: 337.20).

Scheme 18 describes a general method to synthesize the intermediate 125.

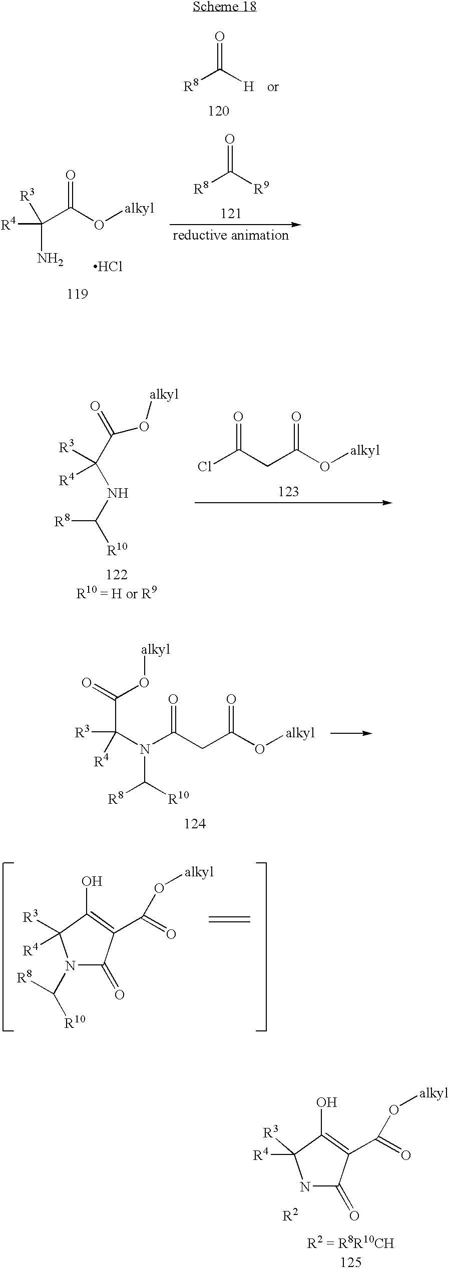

wherein R⁸ and R⁹ include but are not limited to C₁-C₆ alkyl, C₃-C₈ cycloalkyl, aryl, or heterocyclyl.

In this general method, α-aminoacid ester (119) can react with either aldehyde 120 or ketone 121 in reductive amination conditions known in the art to form alkylated aminoacid ester (122). Compound 122 can be acylated with alkyl 3-chloro-3-oxo-propionate (123) with or without base at the temperature range from room temperature to 110° C. in the solvents selected from dioxane, THF, ACN, DMF, DME to form the intermediate 124 which undergoes the cyclization under basic condition such as NaOEt, Et₃N and KO^{t}Bu at the temperature range from room temperature to 100° C. in the solvents selected from EtOH, THF, DMF, DCM to form the desired product (125).

Scheme 19 describes the synthesis of compound 29.

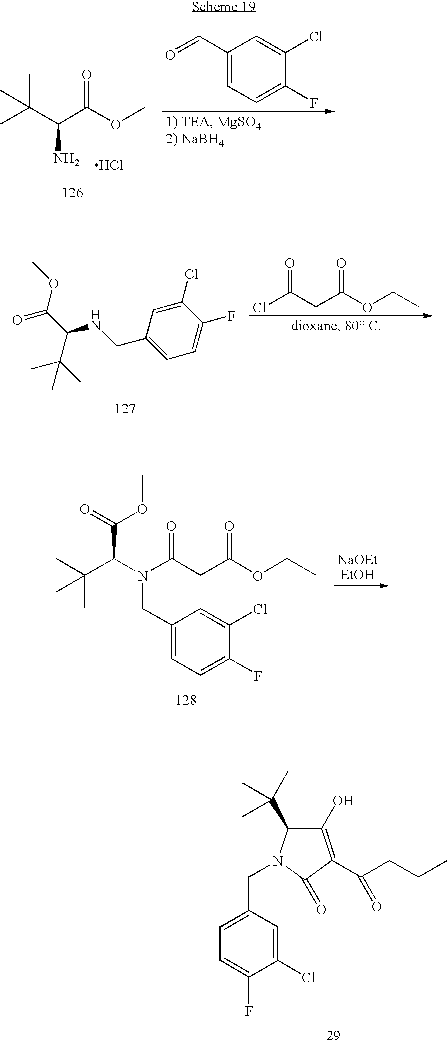

### Synthesis of 2-(3-chloro-4-fluoro-benzylamino)-3,3-dimethyl-butyric acid methyl ester (127)

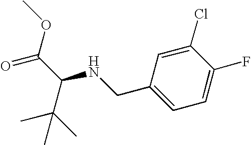

To a stirring mixture of 2-amino-3,3-dimethyl-butyric acid methyl ester (126) (0.5 g, 2.75 mmol) in THF (5.5 mL, 0.5 M), cooled to 0° C. in an ice-bath, was added triethyl amine (TEA) (0.382 mL, 2.75 mmol), MgSO₄ (0.331 g, 2.75 mmol) and finally 3-chloro-4-fluoro-benzaldehyde (0.872 g, 5.5 mmol). The mixture was slowly warmed up to room temperature and continued stirring at room temperature for 12 h. The reaction mixture was then centrifuged and the supernatant was decanted and concentrated to dryness. The residual was resuspended in methanol (9.8 mL, 0.28 M). NaBH₄ (0.208 g, 5.5 mmol) was added portionwise with stirring at room temperature. The reaction mixture was stirred at room temperature for 2 h before quenching in 1N HCl (100 mL). Product was extracted with EtOAc (100 mL), washed with brine and water and concentrated to dryness to afford crude 2-(3-chloro-4-fluoro-benzylamino)-3,3-dimethyl-butyric acid methyl ester (127) (0.856 g). ¹H NMR (400 MHz, CDCl₃) δ: 1.20 (s, 9H), 3.21 (s, 1H), 3.79 (s, 3H), 4.08-4.17 (m, 1H), 4.66 (s, 2H), 7.31 (t, 1H, J=8.6 Hz), 7.78-7.81 (m, 1H), 7.96 (d, 1H, J=7.0 Hz), 9.92 (s, 1H). LC-MS (ESI⁺): m/e 288.1 [M+1]⁺ (exact ms: 287.7).

### Synthesis of 2-[(3-chloro-4-fluoro-benzyl)-(2-ethoxycarbonyl-acetyl)-amino]-3,3-dimethyl-butyric acid methyl ester (128)

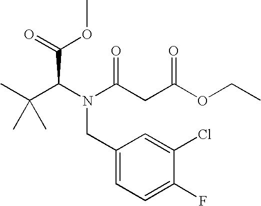

To a solution of 2-(3-chloro-4-fluoro-benzylamino)-3,3-dimethyl-butyric acid methyl ester (127) (0.38 g, 1.32 mmol) in 1,4-dioxane (6.6 mL, 0.2 M), was added chloro ethyl malonate (0.174 mL, 1.38 mmol) and the resulting mixture was heated at 80° C. for 3 h. The reaction mixture was cooled, quenched with saturated aqueous solution of NaHCO₃ (100 mL) and extracted with EtOAc (2×100 mL). The combined organic layer was then concentrated to dryness under reduced pressure and purified by column chromatography on silica gel to afford 2-[(3-chloro-4-fluoro-benzyl)-(2-ethoxycarbonyl-acetyl)-amino]-3,3-dimethyl-butyric acid methyl ester (128) (0.275 g, 52% yield) as a clear, yellow oil. ¹H NMR (400 MHz, CDCl₃) δ: 1.11 (s, 9H), 1.27 (t, 3H, J=7.0 Hz), 3.23 (d, 1H, J=15.7 Hz), 3.32 (d, 1H, J=15.5 Hz), 3.52 (s, 3H), 4.18 (quartet, 2H, J=7.6 Hz), 4.63 (d, 1H, J=17.9 Hz), 5.06 (d, 1H, J=17.9 Hz), 6.97-7.01 (m, 1H), 7.10 (t, 1H, J=8.7 Hz), 7.17-7.19 (m, 1H). LC-MS (ESI⁺): m/e 402.04 [M+1]⁺ (exact ms: 401.1).

### Synthesis of 5-tert-butyl-1-(3-chloro-4-fluoro-benzyl)-4-hydroxy-2-oxo-2,5-dihydro-1H-pyrrole-3-carboxylic acid ethyl ester (29)

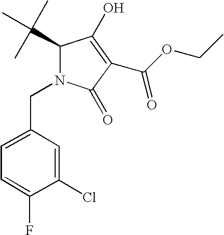

Into a solution of 2-[(3-chloro-4-fluoro-benzyl)-(2-ethoxycarbonyl-acetyl)-amino]-3,3-dimethyl-butyric acid methyl ester (128) (0.275 g, 0.68 mmol) in ethanol (1.5 mL, 0.46 M) was added NaOEt (21 wt % in EtOH, 0.33 mL, 1.03 mmol). The mixture was stirred at room temperature for 17 h, quenched with 1N HCl (100 mL) and extracted with EtOAc (2×100 mL). The combined organic layer was then concentrated to dryness under reduced pressure to afford the crude product of 5-tert-butyl-1-(3-chloro-4-fluoro-benzyl)-4-hydroxy-2-oxo-2,5-dihydro-1H-pyrrole-3-carboxylic acid ethyl ester (29) (0.24 g, 87% yield) as a solid which was directly used in next step without further purification. ¹H NMR (400 MHz, CDCl₃) δ: 1.07 (s, 9H), 1.44 (t, 3H, J=7.0 Hz), 3.61 (s, 1H), 4.24 (d, 1H, J=15.6 Hz), 4.38-4.46 (m, 2H), 5.13 (d, 1H, J=15.6 Hz), 7.04-7.09 (m, 2H), 7.21 (dd, 1H, J₁=7.1 Hz, J₂=2.5 Hz). LC-MS (ESI⁺): m/e 370.1 [M+1]⁺ (exact ms: 369.1).

Scheme 20 describes a general method to synthesize the intermediate 132.

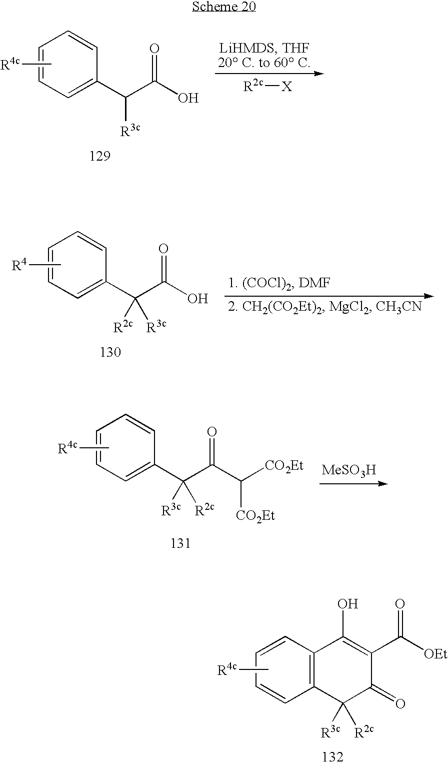

In this general method, compound 129 can be alkylated by R²CX (wherein X is Cl, Br, I, OTs, or OTf) to form compound 130. Carboxylic acid group of compound 130 can be converted to acid chloride, followed by reacting with malonic acid diethyl ester to form compound 131. Compound 131 can undergo intra-molecular cyclization in the presence of methyl sulfonic acid to form intermediate 132.

### EXAMPLE 5-1

### Scheme 21 Describes a Specific Method to Synthesize the Intermediate 33

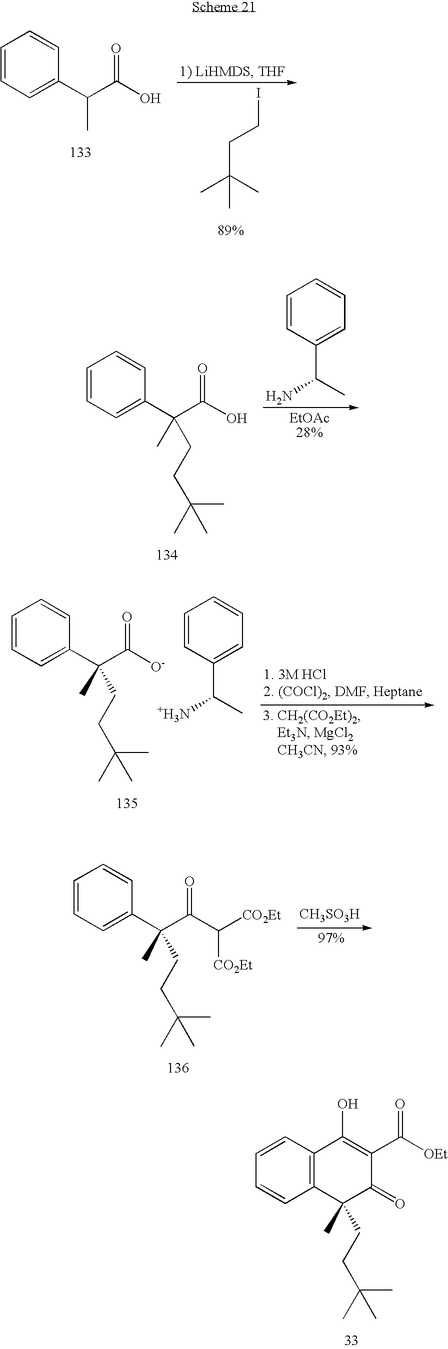

### Synthesis of 2,5,5-trimethyl-2-phenylhexanoic acid (134)

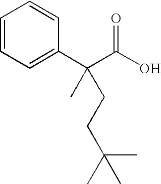

To a 1 M solution of Lithium bis(trimethylsilyl)amide in THF (84.21 mL, 84.21 mmol) was added 2-phenyl propionic acid (133) (4.16 mL, 30.40 mmol) dropwise while maintaining the temperature of the solution below 25° C. Upon completion of the addition, the solution was heated to 50° C., stirred for 20 min., and then cooled down to room temperature. 3,3-Dimethyl-1-iodobutane (8.06 g, 38.01 mmol) was added. The reaction was stirred at 60° C. for 31 h and then cooled to room temperature and concentrated. The residue was dissolved in H₂O (45 mL) and cooled to 10-15° C. The solution was acidified to pH=1-2 via slow addition of 6 M HCl. The resulting mixture was extracted with EtOAc (3×100 mL). The organic layers were combined, washed with brine (100 mL), dried over MgSO₄, filtered and concentrated to afford the desired product, 2,5,5-Trimethyl-2-phenylhexanoic acid (134) (6.34 g, 27.06 mmol, 89%) as a thick oil. ¹H NMR (400 MHz, CDCl₃) δ: 0.88 (9H, s), 1.04-1.18 (2H, m), 1.58 (3H, s), 1.91-2.08 (2H, m), 7.32-7.39 (5H, m).

### Synthesis of (2R)-2,5,5-trimethyl-2-phenylhexanoic acid, (1S)-1-phenylethanamine salt (135)

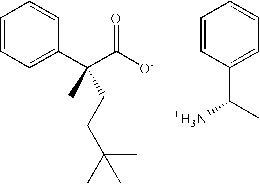

To a solution of 2,5,5-trimethyl-2-phenylhexanoic acid (134) (7.68 g, 32.77 mmol) in EtOAc (172 mL) was added dropwise (S)-α-phenylethanamine (2.92 mL, 22.94 mmol). A suspension was formed. The suspension was stirred at room temperature for 18 h, and filtered. The white solid was re-dissolved in boiling EtOAc (95 mL). The solution was cooled to room temperature and stirred overnight. The suspension was filtered, and the solid was further dried on vacuum to afford (2R)-2,5,5-trimethyl-2-phenylhexanoic acid, (1S)-α-phenylethanamine salt (135) (3.20 g, 9.00 mmol, 27.5%, 98.6 ee) as a white solid. ¹H NMR (400 MHz, DMSO-d₆) δ: 0.84 (9H, s), 0.95-1.09 (2H, m), 2.55 (3H, d, J=6.0 Hz), 1.39 (3H, s), 1.76-1.92 (2H, m), 4.02 (1H, q, J=6.4 Hz), 7.16-7.21 (2H, m), 7.26-7.36 (8H, m).

### Synthesis of 2-[(2R)-2,55-trimethyl-2-phenylhexanoyl]malonate (136)

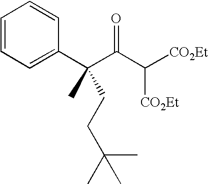

To a solution of (2R)-2,5,5-trimethyl-2-phenylhexanoic acid, (1S)-α-phenylethanamine salt (135) (1.60 g, 4.50 mmol) in EtOAc (10 mL) and water (5 mL) at room temperature was added 3 M HCl dropwise until pH<2. The layers were separated, and the aqueous layer was extracted with EtOAc (2×10 mL). The organic layers were combined, dried over MgSO₄ and filtered. The filtrate was concentrated under vacuum to afford a thick oil. The oil was dissolved in heptane (10 mL), and then DMF (0.37 mL, 4.78 mmol) and (COCl)₂ (1.25 mL, 14.34 mmol) were added sequentially.

The resulting mixture was stirred at room temperature for 2 h, then filtered through a short pad of celite. The filtrate was concentrated and re-dissolved in CH₃CN (7 mL). To a separate flask containing at 0° C. a solution of diethyl malonate (0.73 mL, 4.78 mmol) and MgCl₂ (0.46 g, 4.78 mmol) in CH₃CN (10 mL) was added Et₃N (1.40 mL, 10.04 mmol) dropwise. The mixture was stirred at 0° C. for 15 min, then at room temperature for another 2 h. To this mixture at 0° C. was added the solution of the acyl chloride in CH₃CN via cannula. The reaction was stirred at 50° C. for 18 h and then cooled to room temperature and concentrated. The residue was dissolved in EtOAc (30 mL) and washed with 1N HCl (2×20 mL). The organic layer was dried over MgSO₄, filtered and concentrated to afford a thick oil as the desired product (136). It was used directly for next step.

### Synthesis of ethyl (4R)-4-(3,3-dimethylbutyl)-1-hydroxy-4-methyl-3-oxo-3,4-dihydronaphthalene-2-carboxylate (33)

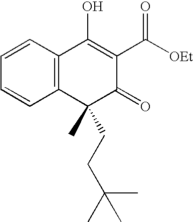

The crude 2-[(2R)-2,5,5-trimethyl-2-phenylhexanoyl]malonate (136) was dissolved in CH₃SO₃H (10 mL) and stirred at room temperature for 3 h. The reaction was cooled to 0° C., then quenched by dropwise addition of cold water. The mixture was extracted with EtOAc (3×20 mL). The organics were combined, dried over MgSO₄, filtered and concentrated. The crude was purified by flash column chromatography (silica, 0-20% EtOAc/Hexane) to afford ethyl (4R)-4-(3,3-dimethylbutyl)-1-hydroxy-4-methyl-3-oxo-3,4-dihydronaphthalene-2-carboxylate (33) (1.34 g, 4.05 mmol, 90% yield over two steps) as a think oil. ¹H NMR (400 MHz, CDCl₃) δ: 0.47-0.60 (1H, m), 0.75 (4.5H, s), 0.77 (4.5H, s), 0.79-0.96 (1H, m), 1.44-1.52 (3H, m), 1.54 (1.5H, s), 1.66 (1.5H, s), 1.74-1.81 (0.5H, m), 1.91-1.99 (0.5H, m), 2.22-2.36 (1H, m), 4.42-4.53 (2H, m), 7.38-7.44 (2H, m), 7.55-7.61 (1H, m), 8.14-8.25 (1H, m).

Compound 132 having different R^{2c} and R^{3c} substituents can be made in an analogous matter to that described in Scheme 21 using appropriate starting reactants.

The synthesis of compound 132 where R^{3c} is —NHR²⁶ can be accomplished using the procedure described in Scheme 22.

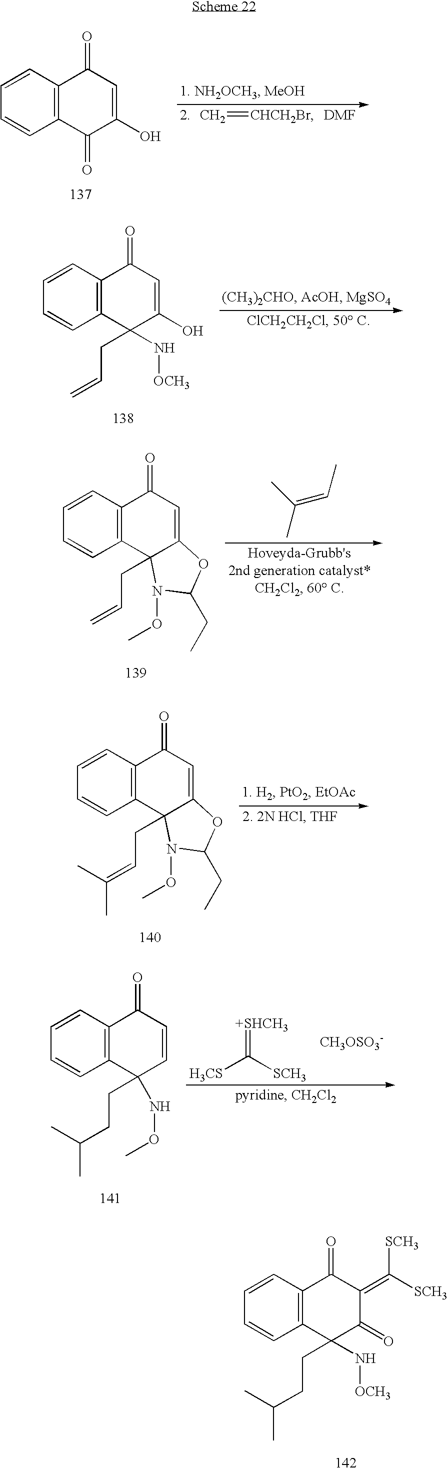

In this specific example, conversion of 2-hydroxy-1,4-naphthoquinone (137) to allyl methoxyamine intermediate 138 can be accomplished by oximation, followed by reaction with allylindium. Compound 138 can be converted to an isopropylidine derivative 139, followed by olefinmetathesis to give prenyl analog 140. Hydrogenation, followed by acid hydrolysis gives isoamyl intermediate 141, which can be converted to dithioketeneacetal 142 using the condition shown in Scheme 18.

### Biological Testing

The ability of compounds of Formula I to inhibit HCV replication can be demonstrated in the following in vitro assays.

Compounds were tested for HCV NS5B polymerase inhibition. Assays were performed in a 96-well streptavidin-coated FlashPlate using 20 nM enzyme, 1.4 μCi of [(α-³³P]GTP, 0.63 μM GTP, and 250 nM 5′biotinylated oligo (rG₁₃)/poly rC in 20 mM Tris-HCl, pH 7.5, 5 mM MgCl₂, 5 mM dithiothreitol, 0.1 g/L bovine serum albumin, and 100 U/ml RNAse inhibitor. The reaction was stopped by aspiration after 75 min at 28° C. and the plate was washed several times. After washing and drying the plate, incorporated radioactivity was counted using a Microbeta scintillation counter. IC₅₀ values were calculated relative to the uninhibited control and inhibition data were fitted to a 4-parameter IC₅₀ equation. For very potent inhibitors, the data were fitted to a tight binding quadratic equation to obtain IC₅₀ values.

Tested compounds 22, 23, 24, 28, 32, 36, 37, 38, 54, 63, 68, and 75 exhibited NS5B polymerase inhibition with IC₅₀ values less than 100 μM (“+” or “++” in Table 1). Tested compounds 22, 23, 28, 32, 36, 37, 38, 54, 63, and 75 exhibited NS5B polymerase inhibition with IC₅₀ values less than 1 μM (“++” in Table 1).

TABLE 1

| Compound No. | NS5B IC₅₀ |
|---|---|
| 22 | ++ |
| 23 | ++ |
| 24 | + |
| 28 | ++ |
| 32 | ++ |
| 36 | ++ |
| 37 | ++ |
| 38 | ++ |
| 54 | ++ |
| 63 | ++ |
| 68 | + |
| 75 | ++ |

### HCV Replicon Assay (Replicon EC₅₀ (μM))

The cell culture component of the assay is performed essentially as described by Bartenschlager et al., (Hepatology, 35, 694-703. (2002)), wherein exponentially growing HCV Huh-7/C24 replicon cells are seeded at 4.5×10³ cells/well in 96 well plates and 24 hours later are treated with eight point half-log concentration of compound. After 72 hours exposure the media is discarded from the compound assay plate and the cell monolayers are lysed by addition of 150 μL lysis mixture (Genospectra) with incubation at 53° C. for 45 minutes. Following incubation, each lysate is thoroughly mixed and 5 μL (NS3 probe) or 10 μL (GAPDH probe) of each lysate is then transferred to the capture plate and analyzed by branched DNA (bDNA) assay.

### Branched DNA Assay

Based on sequences provided for NS3 [AJ242652], Genospectra (Fremont, Calif., USA) designed and synthesized probes to these analytes (together with GAPDH). Cellular bDNA analysis is carried out essentially as described in the Genospectra protocol (details in Shyamala, V. et al, Anal Biochem, 266, 140-7 (1999)), wherein target specific capture extenders, label extenders and blocking probes are added to the capture plate after the addition of 5 or 10 μL cell lysate. After annealing overnight, during which the target RNA is captured to the plate via interaction with the capture extenders, the plate is washed, and then amplifier (which binds via the label extenders) and label probe are sequentially added. After subsequent addition of the chemilumigenic substrate (dioxetan), each plate is read by luminometer (Wallac 1420 Multilabel HTS Counter Victor 2). The luminescence signal is proportional to the amount of mRNA present in each lysate. In addition to the samples, cell lysate only (no probe) background controls are also included on each bDNA assay plate and the average signal from these control wells is subtracted from the sample reading prior to analysis. Percent of no drug control is determined for both the NS3 and GAPDH signals for each compound also. % Inhibition is determined for each compound concentration in relation to the no drug control to calculate the EC₅₀.

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention.

What is claimed is:

1. A compound of Formula I 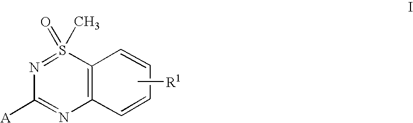
wherein
A is selected from the group consisting of 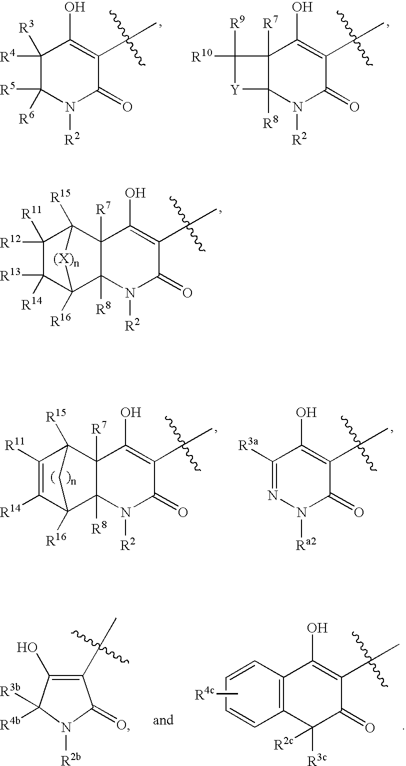
wherein
R¹ is 1-4 moieties selected independently from H, halo, nitro, aryl, heterocyclyl, —CHR⁷—S(O)₂R¹⁸, NR⁸R⁹, —NR⁷S(O)₂R¹⁸, and —NR¹⁷S(O)₂NR^{18a}R^{19a}, wherein R¹⁷, R¹⁸, R¹⁹, R^{18a}, and R^{19a}, are independently H, C₁-C₆ alkyl, C₃-C₈ cycloalkyl, aryl, or heterocyclyl, or R^{18a} and R^{19a} combine with the atom(s) to which they are attached to form a 5- or 6-membered heterocyclyl ring,
R² is H, C₁-C₈ alkyl, C₂-C₆ alkenyl, C₁-C₆ alkylamine, C₁-C₆ dialkylamine, C₃-C₈ cycloalkyl, —C₁-C₆ alkylene(C₃-C₈ cycloalkyl), —C₁-C₆ alkylene(aryl), —C₁-C₆ alkylene(heterocyclyl), aryl, or heterocyclyl,
R³, R⁴, R⁵ and R⁶ are independently H, halo, cyano, hydroxyl, amino, C₁-C₆ alkyl, C₁-C₆ alkoxy, C₁-C₆ hydroxyalkyl, C₂-C₆ alkenyl, C₂-C₆ alkynyl, C₁-C₆ alkylamine, C₁-C₆ dialkylamine, C₃-C₈ cycloalkyl, C₁-C₆ alkylene(cycloalkyl), C₁-C₆ alkylene(aryl), C₁-C₆ alkylene(heterocyclyl), aryl, heterocyclyl, or R³ and R⁴ or R⁵ and R⁶ can combine with the atom(s) to which they are attached to form a 3- to 6-membered cycloalkyl spiro ring, or R² and R⁵, or R² and R⁶ can combine with the atom(s) to which they are attached to form a 5- or 6-membered heterocyclyl ring,
Y is (CR²⁰R²¹)ₘ, wherein m is 2, 3, 4 or 5,
n is 1 or 2,
wherein when n is 1, X is O or —CR²²R²³, and when n is 2, X is —CR²²R²³—CR²⁴R²⁵, R⁷, R⁸, R⁹, R¹⁰, R¹¹, R¹², R¹³, R¹⁴, R¹⁵, R¹⁶, R²⁰, R²¹, R²², R²³, R²⁴ and R²⁵ are independently H, C₁-C₆ alkyl, or halo, or R⁷ and R⁹ combine with the atoms to which they are attached to form a 3-membered cycloalkyl ring, or R²² and R²⁴ combine with the atoms to which they are attached to form a 3-membered cycloalkyl ring,
R^{2a} is C₁-C₆ alkyl, C₃-C₈ cycloalkyl, C₁-C₆ alkylene(C₃-C₈ cycloalkyl), C₁-C₆ alkylene(aryl), C₁-C₆ alkylene(heterocyclyl), aryl, or heterocyclyl,
R^{3a} is H, halo, C₁-C₆ alkyl, C₁-C₆ alkoxy, C₁-C₆ haloalkyl, C₁-C₆ hydroxyalkyl, C₂-C₆ alkenyl, C₃-C₈ cycloalkyl, aryl, heterocyclyl, NR¹⁸R¹⁹,
R^{2b} is C₁-C₇ alkyl, C₂-C₆ alkenyl, C₃-C₈ cycloalkyl, C₁-C₆ alkylene(C₃-C₈ cycloalkyl), C₁-C₆ alkylene(aryl), C₁-C₆ alkylene(heterocyclyl), aryl, or heterocyclyl,
R^{3b} and R^{4b} are independently selected from H, C₁-C₆ alkyl, C₃-C₈ cycloalkyl, C₁-C₆ alkylene(aryl), aryl, heterocyclyl, or R^{3b} and R^{4b} combine with the C atom to which they are attached to form a 3-, 4-, 5- or 6-membered heterocyclyl ring that may contain heteroatoms such as N on the ring,
R^{2c} and R^{3c} are independently H, C₁-C₆ alkyl, C₁-C₆ alkenyl, C₁-C₆ haloalkyl, C₁-C₆ hydroxyalkyl, C₁-C₆ alkylene(C₃-C₈ cycloalkyl), C₁-C₆ alkylene(aryl), or C₁-C₆ alkylene(heterocyclyl) having 1, 2, or 3 N, O, or S atoms, or R^{2c} and R^{3c} combine together with the carbon atom to which they are attached to form a monocyclic ring selected from the group consisting of cycloalkyl and cycloalkenyl, or R^{3c} is —NHR²⁶ wherein R²⁶ is selected from C₁-C₆ alkyl, C₁-C₆ alkylene(aryl), C₁-C₆ alkoxy, C₁-C₆ alkylene-CO₂R⁸, —COR⁸, —CO₂R⁸, —CONH₂ and —SO₂R¹⁸,
R^{4c} is independently H, halo, OH, CN, C₁-C₆ alkyl, C₁-C₆ alkoxy, C₁-C₆ haloalkyl, C₁-C₆ hydroxyalkyl, C₂-C₆ alkenyl, C₂-C₆ alkynyl, —COOH, —CONR^{18a}R^{19a} and —COOR¹⁸,
wherein the above alkyl, alkylene, alkenyl, alkynyl, aryl, cycloalkyl, or heterocyclyl moieties provided in R¹, R², R^{2a}, R^{2b}, R^{2c}, R³, R^{3a}, R^{3b}, R^{3c}, R⁴, R^{4b}, R^{4c}, R⁵, R⁶, R⁷, R⁸, R⁹, R¹⁰, R¹¹, R¹², R¹³, R¹⁴, R¹⁵, R¹⁶, R¹⁷, R¹⁸, R^{18a}, R¹⁹, R^{19a}, R²⁰R²¹, R²², R²³, R²⁴, R²⁵ and R²⁶ are each optionally and independently substituted by 1-3 substituents selected from
alkylamine,
amino,
aryl, cycloalkyl, heterocyclyl,
C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms,
carboxyl,
cyano,
halo,
hydroxy,
nitro,
—C(O)OH, —C(O)₂—(C₁-C₆ alkyl), —C(O)₂—(C₃-C₈ cycloalkyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkyl), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkylene)cycloalkyl,
wherein each of the above optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₂-C₆ alkenyl, and C₁-C₆ hydroxyalkyl, wherein each alkyl is optionally substituted by one or more halo substituents, or a pharmaceutically acceptable salt, hydrate, tautomer or stereoisomer thereof.

2. The compound of claim 1, wherein R¹ is H or NR¹⁷S(O)₂R¹⁸, wherein R¹⁷ and R¹⁸ are independently H, C₁-C₆ alkyl, C₃-C₈ cycloalkyl, aryl, or heterocyclyl, or R¹⁷ and R¹⁸ combine with the atom(s) to which they are attached to form a 5- or 6-membered heterocyclyl ring.

3. The compound of claim 1, wherein R¹ is selected from the group consisting of:

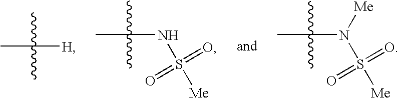

4. The compound of claim 1, wherein R² is selected from the group consisting of C₁-C₆ alkyl, C₃-C₈ cycloalkyl, C₁-C₆ alkylene(C₃-C₈ cycloalkyl), C₁-C₆ alkylene(aryl), and C₁-C₆ alkylene(heterocyclyl).

5. The compound of claim 1, wherein R² is selected from the group consisting of

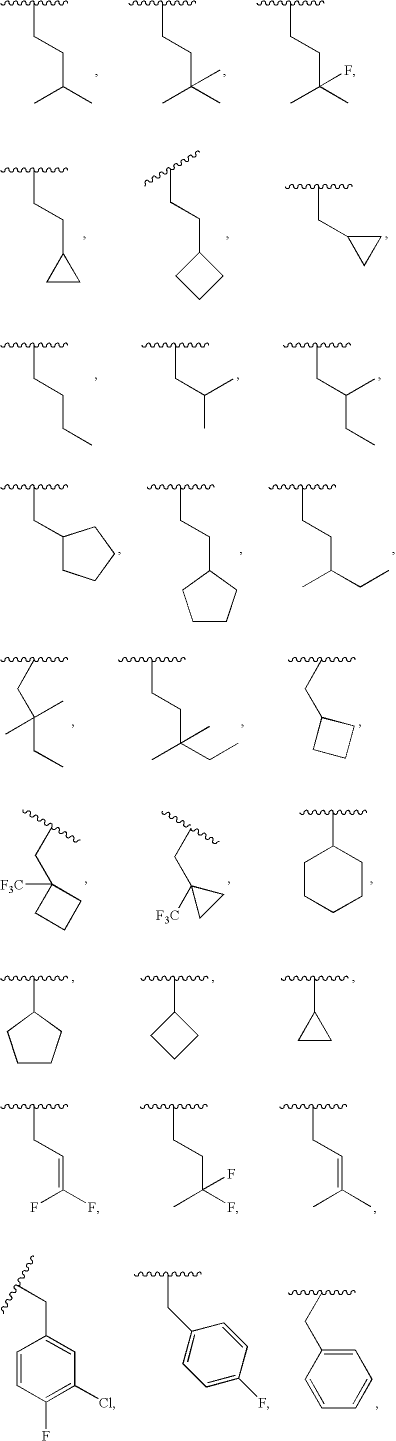

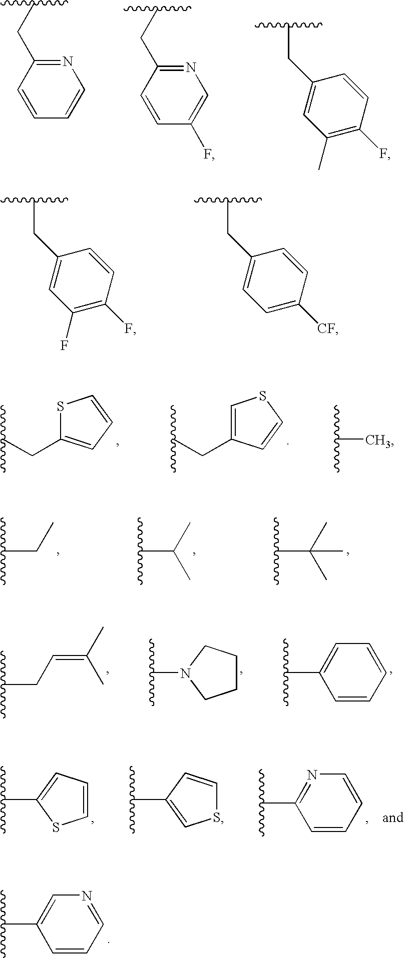

6. The compound of claim 5, wherein R² is selected from the group consisting of

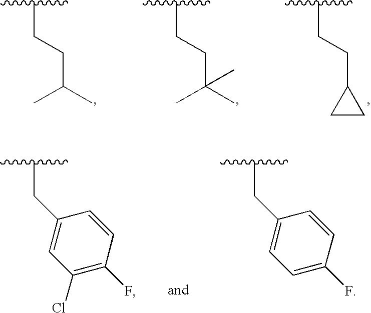

7. The compound of claim 1, wherein R³, R⁴, R⁵, and R⁶ are independently selected from the group consisting of C₁-C₆ alkyl, C₃-C₈ cycloalkyl, aryl, and heterocyclyl.

8. The compound of claim 1, wherein R³, R⁴, R⁵, and R⁶ are independently selected from the group consisting of

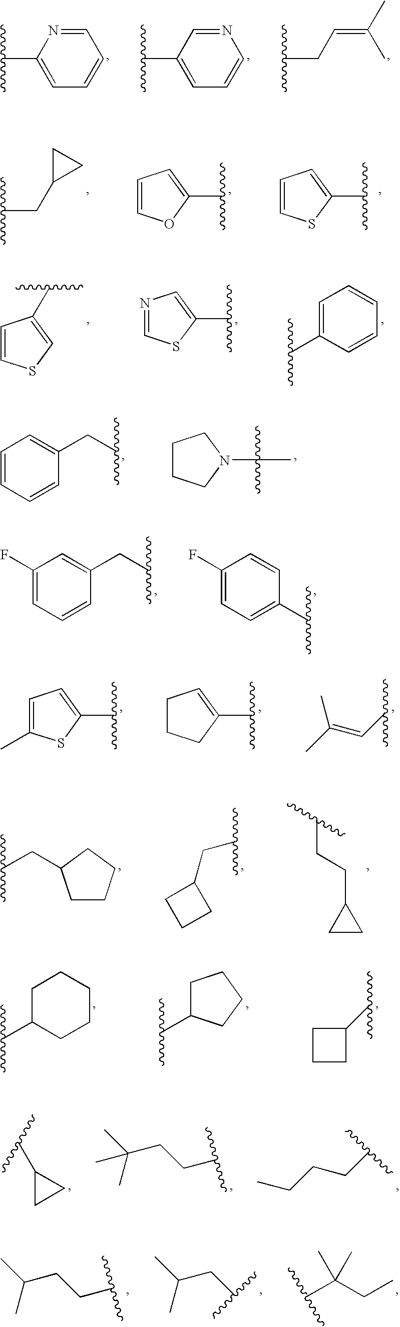

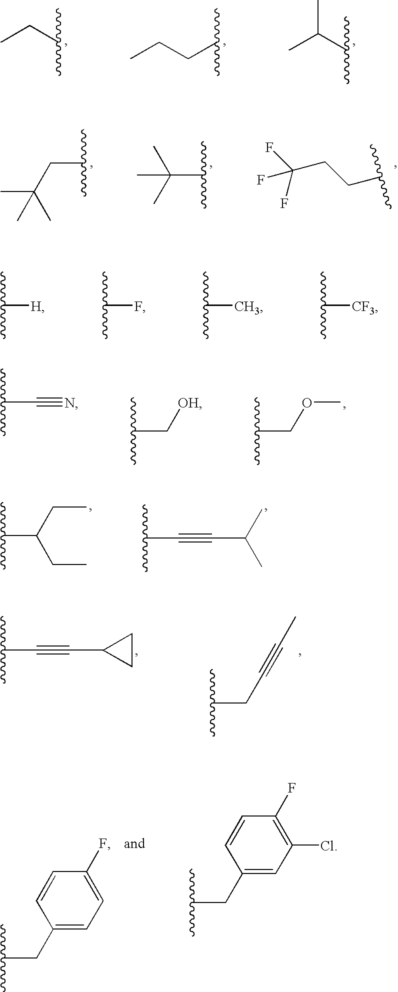

9. The compound of claim 1, wherein R³ and R⁴ or R⁵ and R⁶ can combine with the atom(s) to which they are attached to form

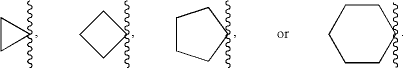

10. The compound of claim 8, wherein R³, R⁴, R⁵ and R⁶ are independently selected from the group consisting of

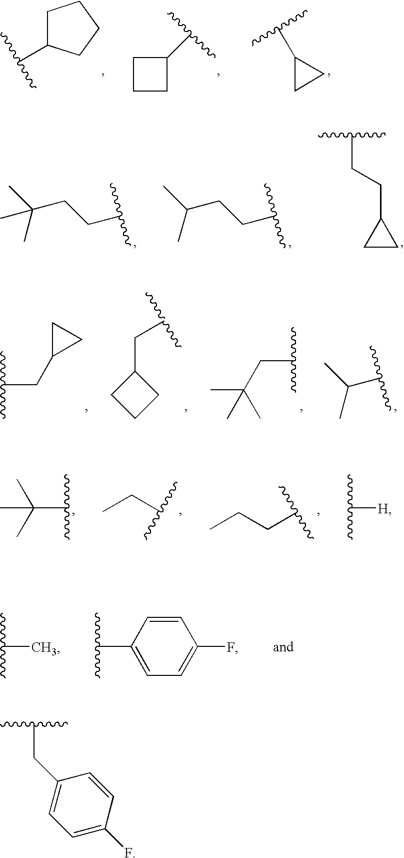

11. The compound of claim 1, wherein R⁷, R⁸, R⁹, R¹⁰, R¹¹, R¹², R¹³, R¹⁴, R¹⁵, and R¹⁶ are independently selected from the group consisting of H and Me.

12. The compound of claim 1, wherein R^{2a} is selected from the group consisting of C₁-C₆ alkyl, C₃-C₈ cycloalkyl, C₁-C₆ alkylene(C₃-C₈ cycloalkyl), C₁-C₆ alkylene(aryl), and C₁-C₆ alkylene(heterocyclyl).

13. The compound of claim 1 wherein R^{2a} is selected from the group consisting of

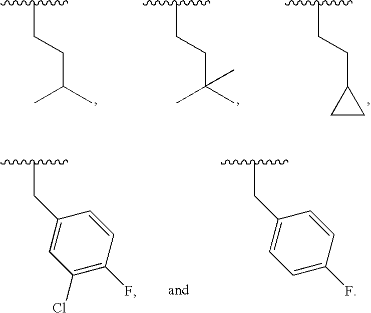

14. The compound of claim 1 wherein R^{3a} is selected from the group consisting of C₁-C₆ alkyl, C₃-C₈ cycloalkyl, aryl, and heterocyclyl.

15. The compound of claim 1 wherein R^{3a} is selected from the group consisting of

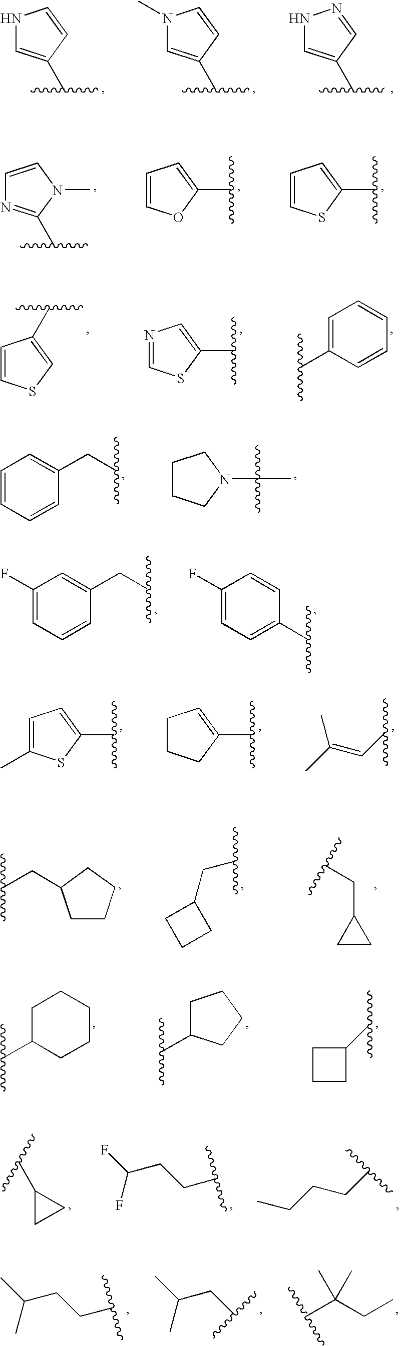

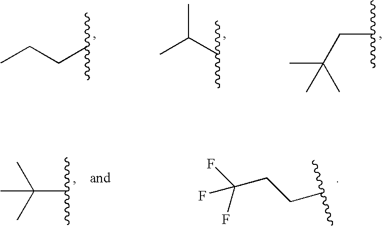

16. The compound of claim 15 wherein R^{3a} is selected from the group consisting of

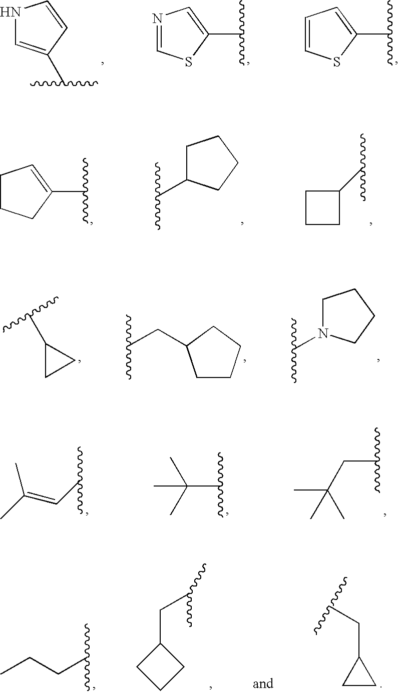

17. The compound of claim 1 wherein R^{2b} is selected from the group consisting of C₁-C₆ alkyl, C₃-C₈ cycloalkyl, C₁-C₆ alkylene(C₃-C₈ cycloalkyl), C₁-C₆ alkylene(aryl), and C₁-C₆ alkylene(heterocyclyl).

18. The compound of claim 1 wherein R^{2b} is selected from the group consisting of

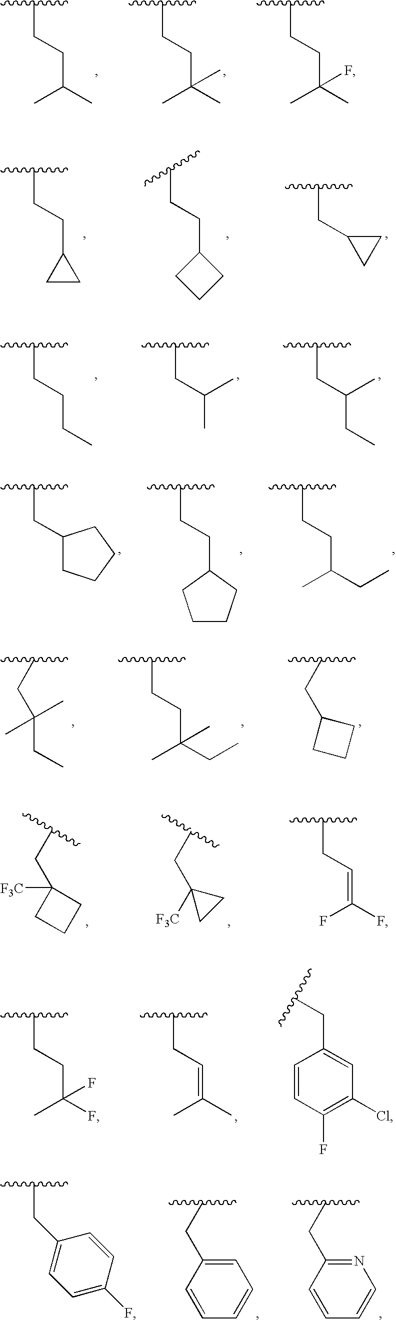

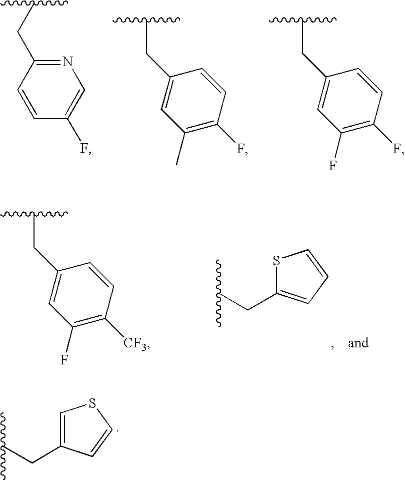

19. The compound of claim 18 wherein R^{2b} is selected from the group consisting of

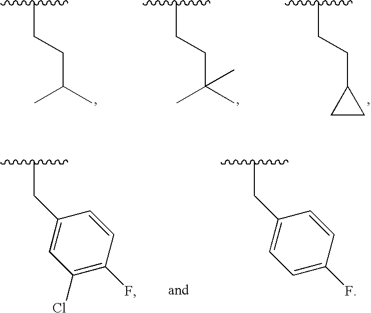

20. The compound of claim 1 wherein R^{3b} and R^{4b} are independently selected from the group consisting of C₁-C₆ alkyl, C₃-C₈ cycloalkyl, aryl, and heterocyclyl.

21. The compound of claim 1 wherein R^{3b} and R^{4b} are independently selected from the group consisting of

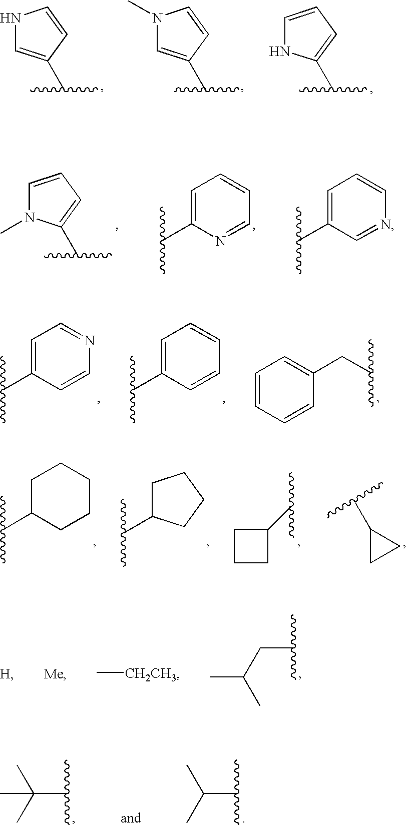

22. The compound of claim 21 wherein R^{3b} and R^{4b} are independently selected from the group consisting of

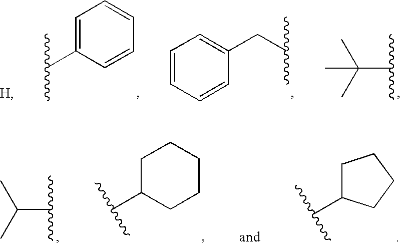

23. The compound of claim 1 wherein R^{2c} and R^{3c} are independently selected from the group consisting of C₁-C₆ alkyl, C₁-C₆ alkylene(C₃-C₈ cycloalkyl), C₁-C₆ alkylene(aryl), and C₁-C₆ alkylene(heterocyclyl) having 1, 2, or 3 N, O, or S atoms.

24. The compound of claim 1 wherein R^{2c} and R^{3c} are independently selected from the group consisting of

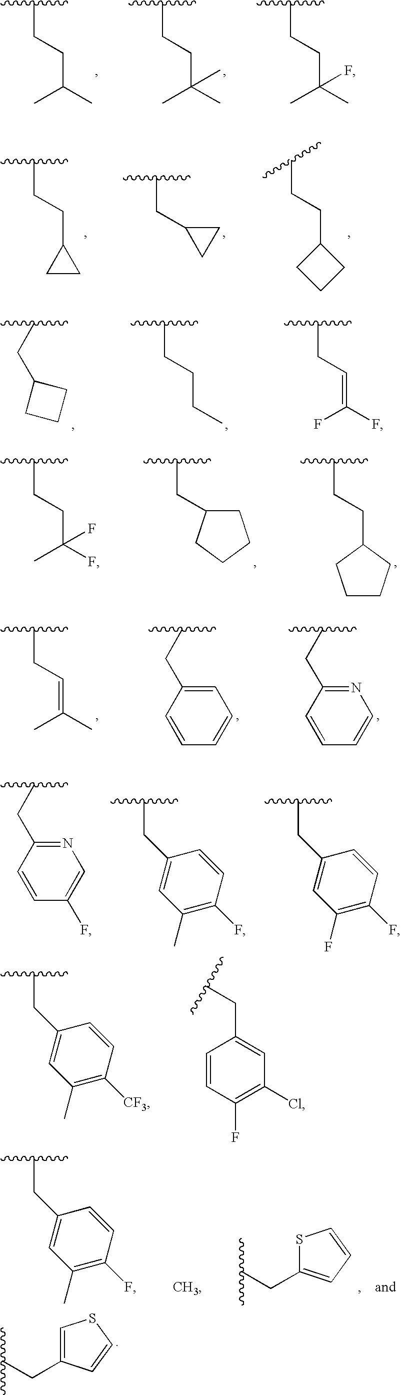

25. The compound of claim 24 wherein R^{2c} and R^{3c} are independently selected from the group consisting of

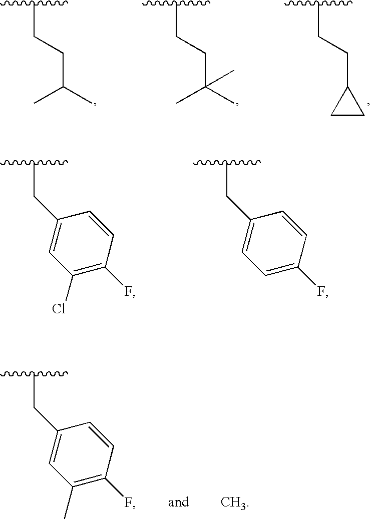

26. The compound of claim 1 wherein R^{4c} is selected from the group consisting of H, halo, OH, CN, C₁-C₆ alkyl, —CONR^{18a}R^{19a} and —COOR¹⁸.

27. The compound of claim 1 wherein R^{4c} is selected from the group consisting of H, F, CH₃, OH, CN, —COOH, —CO₂Me and —CONH₂.

28. The compound of claim 27 wherein R^{4c} is selected from the group consisting of H, F and OH.

29. The compound of claim 1, wherein A is selected from the group consisting of

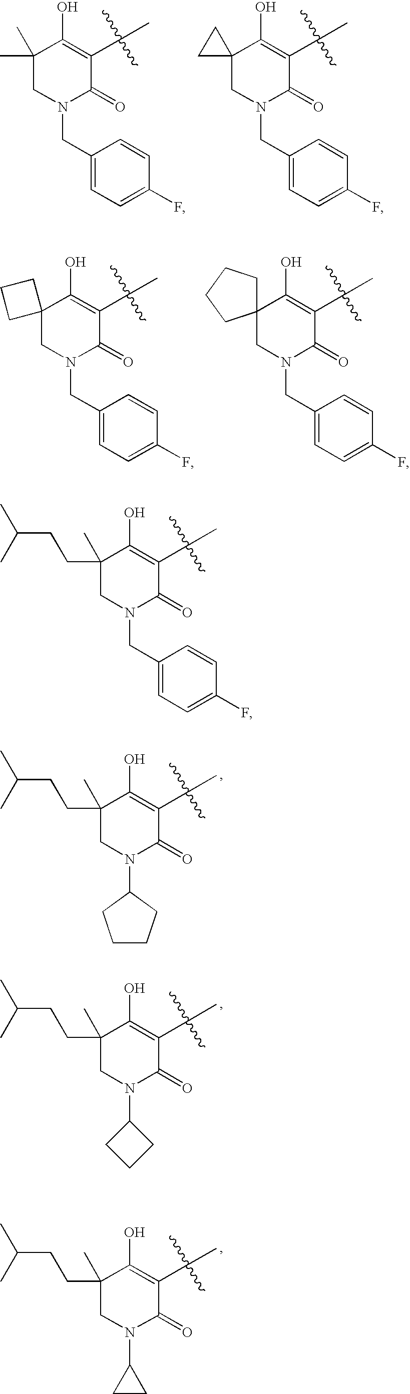

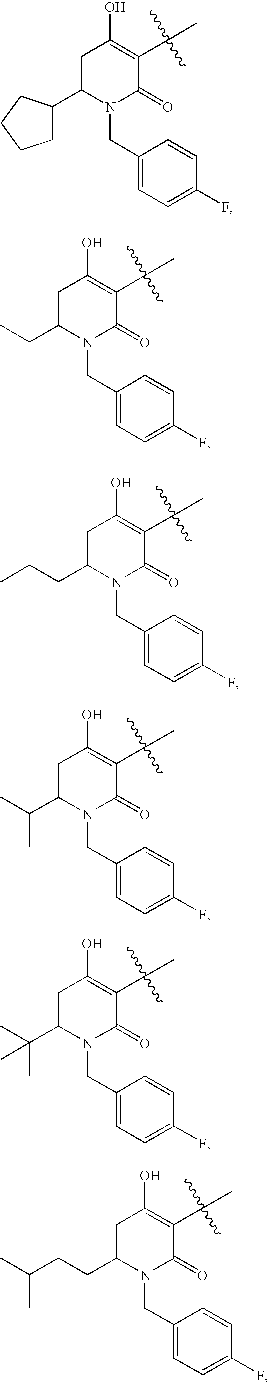

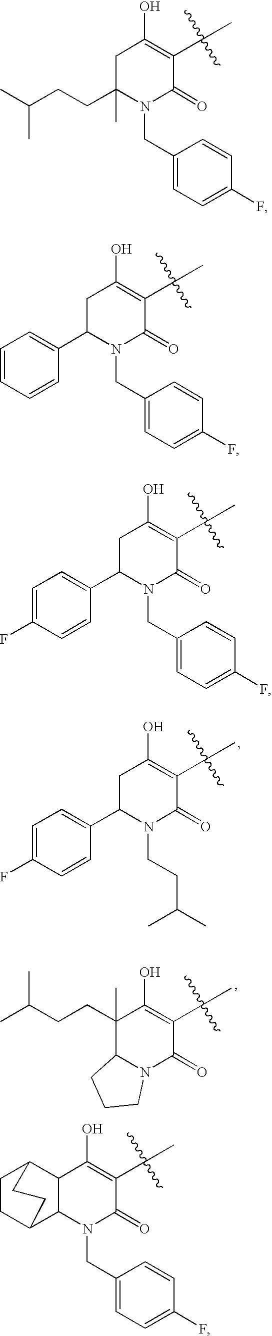

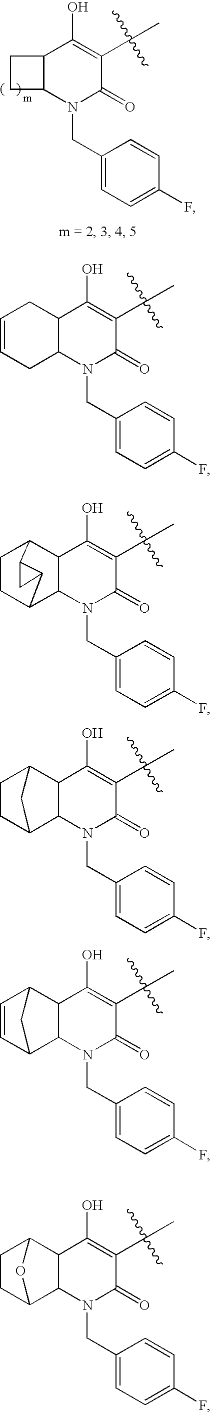

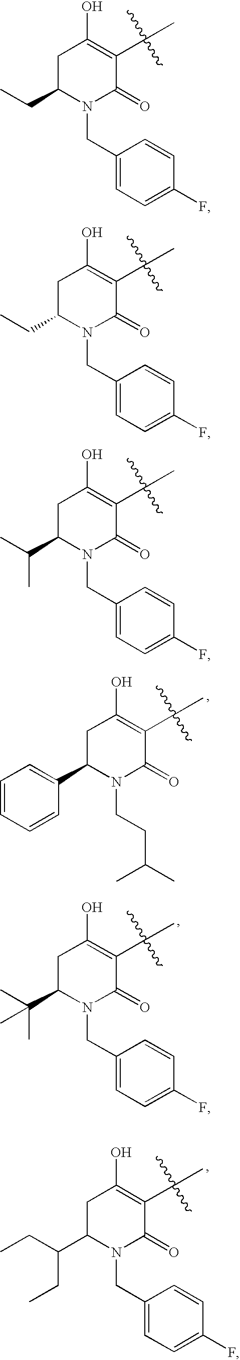

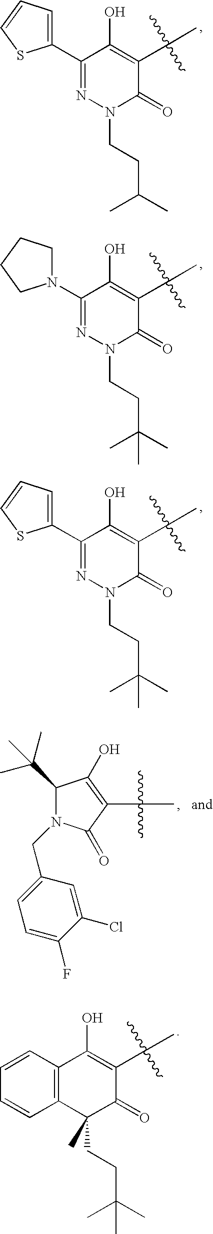

30. A compound selected from the group consisting of

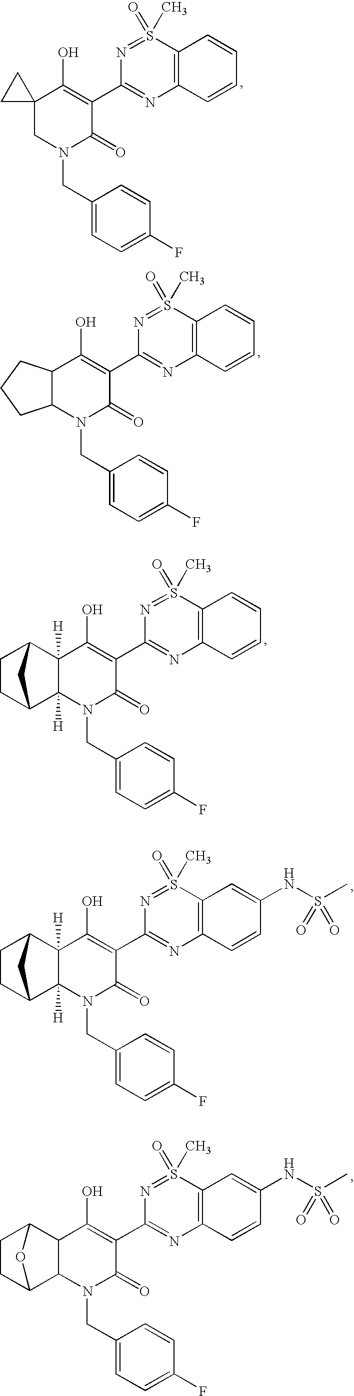

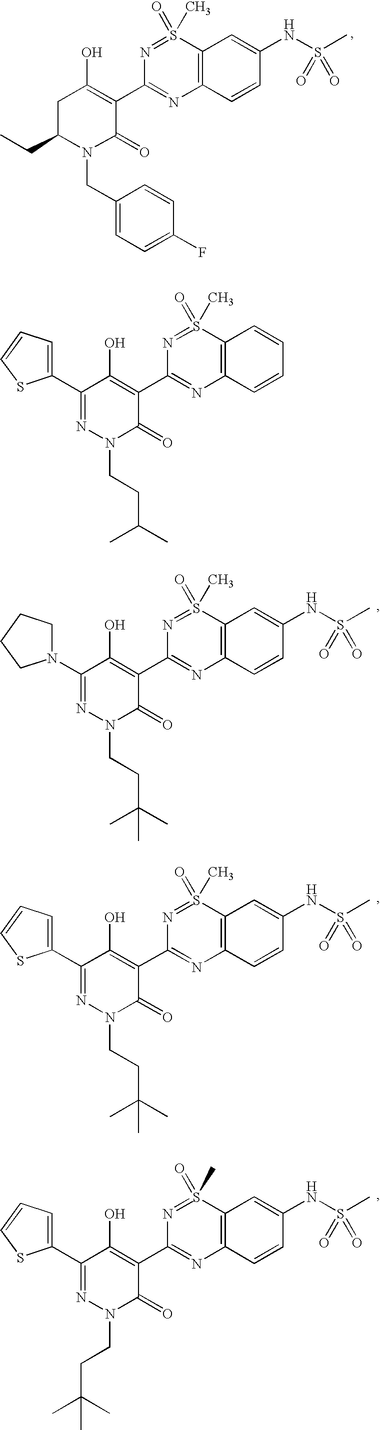

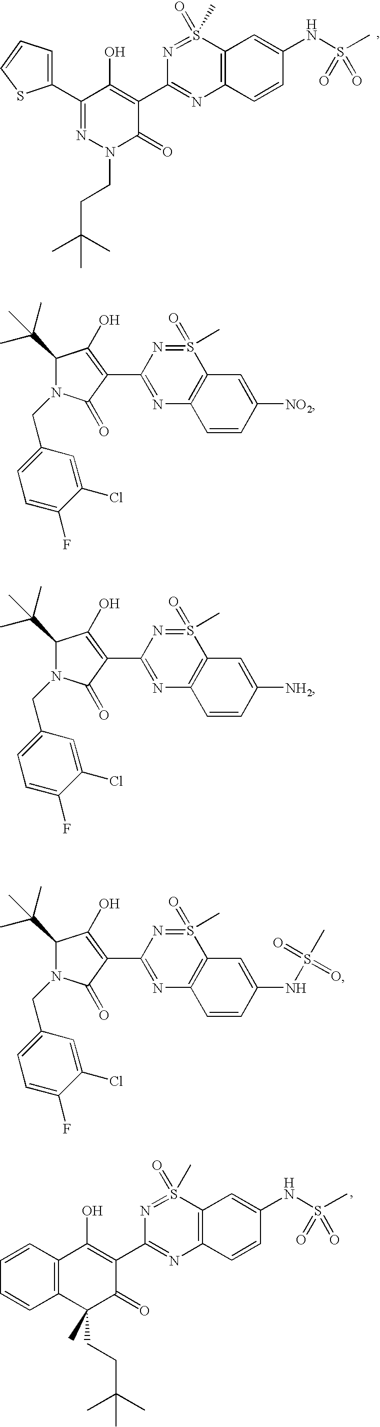

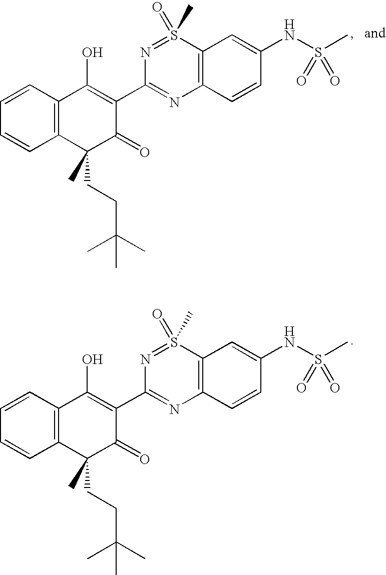

31. The compound of claim 1, wherein the A-ring is selected from the group consisting of:

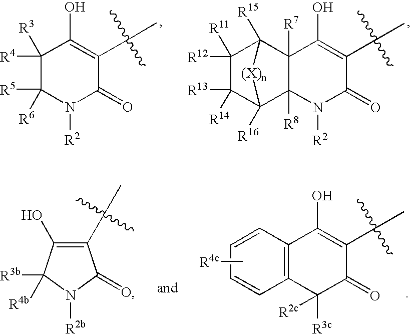

32. A pharmaceutically acceptable composition comprising a compound of claim 1 or a pharmaceutically acceptable salt thereof or pharmaceutically acceptable solvate thereof.

33. A method of inhibiting hepatitis C virus replication comprising exposing hepatitis C virus to a therapeutically effective amount of a compound of claim 1.

34. The method of claim 33 wherein the inhibition of replication occurs in the presence of an additional therapeutic agent selected from the group consisting of an antibiotic, an antiemetic agent, an antidepressant, an antifungal agent, an anti-inflammatory agent, an antiviral agent, an anticancer agent, an immunomodulatory agent, an α-interferon, a β-interferon, a ribavirin, an alkylating agent, a hormone, a cytokine and a toll-like receptor modulator.
